(19) United States
(12) Patent Application Publication
(10) Pub. No.: US 2014/0044713 A1
(43) Pub. Date: Feb. 13, 2014

(54) Compounds

(71) Applicants: Willibrordus Barend Maria De Lau, Utrecht (NL); Johannes Carolus Clevers, Utrecht (NL)

(72) Inventors: Willibrordus Barend Maria De Lau, Utrecht (NL); Johannes Carolus Clevers, Utrecht (NL)

(73) Assignee: KONINKLIJKE NEDERLANDSE AKADEMIE VAN WETENSCHAPPEN, Utrecht (NL)

(21) Appl. No.: 14/111,091

(22) PCT Filed: Apr. 16, 2012
(86) PCT No.: PCT/EP12/56950

§ 371 Date: Oct. 31, 2013

Related U.S. Application Data
(60) Provisional application No. 61/517,191, filed on Apr. 14, 2011.

(30) Foreign Application Priority Data
Apr. 14, 2011 (GB) 1106395.5

(51) Int. Cl. C07K 16/22; G01N 33/50; A61K 45/06; A61K 38/18; A61K 39/395; C07K 14/475; C12N 5/071

(52) U.S. Cl. CPC C07K 16/22; C07K 14/475; G01N 33/5008; C12N 5/0697; A61K 38/18; A61K 39/3955; A61K 45/06
USPC 424/134.1; 530/399; 530/389.2; 435/375; 435/7.1; 435/1.1; 530/387.9; 530/387.3; 514/7.6; 424/145.1

(57) ABSTRACT

The present invention relates to compounds that act as agonists of the Wnt signalling pathway, compositions comprising these compounds and the uses of these compounds, both therapeutic and in research. The invention also provides methods of identifying compounds that act as agonists of the Wnt signalling pathway.

All documents cited herein are incorporated by reference in their entirety.

### TECHNICAL FIELD

The present invention relates to compounds that act as agonists of the Wnt signalling pathway, compositions comprising these compounds and the uses of these compounds, both therapeutic and in research. The invention also provides methods of identifying compounds that act as agonists of the Wnt signalling pathway.

### BACKGROUND

The Wnt signalling pathway plays key roles in diverse biological processes from development to tissue self-renewal and cancer. Wnt signals ultimately activate transcriptional programs that are known to be important in the maintenance and activation of stem cells, in promoting cellular proliferation, and controlling tissue expansion and cell fate determination¹. Loss of components of the Wnt pathway can produce dramatic phenotypes that affect a wide variety of organs and tissues including, but not limited to the gut, hair follicles, hematopoietic system, and bone.

The canonical Wnt/β-catenin signalling pathway is characterised by a series of events that occur when a Wnt protein binds to a cell-surface receptor of a Frizzled (Frz) receptor family member in complex with an LRP co-receptor family member. This results in the activation of Disheveled family proteins which inhibit a complex of proteins that includes axin, GSK-3, and the protein APC to degrade intracellular β-catenin. The resulting enriched nuclear β-catenin enhances transcription by TCF/LEF family transcription factors of a wide range of target genes including Lgr5, transcription of which is up-regulated by the Wnt pathway in colon cancer^{2,3}.

Rspondin (roof plate-specific spondin) proteins are also known to activate β-catenin signalling⁴ and to drive proliferation of cells in the gut⁵. However, Rspondin proteins do not bear sequence similarity to Wnt proteins, are not thought to bind to Frz receptors, and their receptor and mechanism for activating β-catenin signalling is unknown.

Alterations in Wnt and components of the Wnt signalling pathway are known to be associated with carcinogenesis. In view of the key role played by the Wnt signalling pathway in cancer, attempts have been made to develop antagonists of the pathway that may be useful in the treatment of cancer.

As the role of the Wnt signalling in cell growth and proliferation is elucidated, there is also significant interest in developing agonists that enhance the Wnt pathway for treating diseased or damaged tissue, for tissue regeneration and cell growth and proliferation, and tissue engineering.

Wnt/β-catenin signaling is known to increase proliferation of cells in the intestinal crypts⁶.

In addition, Wnt/β-catenin signaling is known to increase bone mass by increasing the number of osteoblasts, cells which play a critical role in bone formation. Thus, it has been suggested that enhancing Wnt/β-catenin signaling to increase the number of osteoblasts, has the potential to provide novel therapeutic approaches for osteoporosis and related bone diseases⁷.

Recently, it was demonstrated that agonising Wnt signalling, could protect against intestinal stem cell damage during graft-versus-host disease, a major complication during bone-marrow transplantation⁸.

Wnt/β-catenin signalling has also been shown to promote hair follicle growth⁹ by promoting the proliferation of matrix cells¹⁰. This suggests that the use of Wnt agonists may also be useful for treatment of baldness or prevention of hairloss during chemotherapy.

Wnt agonists also form a key component of stem cell culture media¹¹. Therefore, controlled modulation of the Wnt/β-catenin signalling pathway could play an important role in improving culture media for cell growth and tissue engineering.

These are just a few examples of the many functions of the Wnt/β-catenin signalling pathway.

Known agonists of the Wnt/β-catenin signalling pathway include the Rspondin proteins (for example NU-206, Kirin Brewery Co Ltd), and small-molecule inhibitors of GSK-3 such as Stemolecule™ BIO (Stemgent®) and those shown in Table 4 of Rey and Ellies Developmental Dynamics 2010 (239) 102-114, Norrin (also called Norrie Disease Protein, or NDP) which is a secreted regulator protein that binds with high affinity to Frizzled-4 and induces activation of the canonical Wnt signaling pathway¹², as well as soluble Wnt proteins and small molecules that mimic Wnt proteins (such as SC-222416).

A problem associated with these known agonists is that they can activate virtually all cells since, for example, GSK3 is ubiquitously distributed. A further disadvantage of using wild-type Rspondin as an agonist, may be that it contains regulatory sequences which could cause it to be activated or inactivated, making dose response predictions very difficult. It is thus difficult to direct agonistic activity to specific cell types and/or tissues and side effects are expected to be widely distributed. There is a need for compounds that can trigger activation of the canonical Wnt pathway in a more selected way without undesired side effects. There is also a need for compounds that can trigger activation of the canonical Wnt pathway in a more predictable manner for safe and predicable dosing regimes.

There is clearly a need to identify and develop further agonists of Wnt/β-catenin signalling for therapeutic reasons, as well as for tissue engineering and cell culture.

### SUMMARY OF INVENTION

Accordingly, the invention provides an agonist of the Wnt pathway which mimics the activity of an Rspondin protein binding to an Lgr protein.

Surprisingly, the inventors have found that the Lgr protein family members, Lgr4, Lgr5 and Lgr6, are facultative Wnt receptor components that mediate Rspondin signaling. In particular, the inventors have established that the Lgr proteins form a complex with Frizzled receptors and LRP proteins to which both Wnt and Rspondin bind. Binding of Rspondin to the Lgr proteins in the complex promotes Wnt/β-catenin signalling in the presence of Wnt. The inventors have shown that Lgr proteins can occur in a physical complex with Frizzled receptors and LRP proteins and bind soluble Rspondins. Engagement of Lgr proteins (Lgr4 or Lgr5 or Lgr6) by Rspondin proteins triggers downstream canonical Wnt signals through the associated Frizzled-LRP complex in the presence of Wnt. It is postulated that binding of Wnt to Frizzled receptors and LRP proteins within the complex induces conformational or biochemical changes in the receptor complex that are essential for the subsequent enhancement of signalling activity by Rspondin/Lgr interaction.

The mechanism for Rspondin-mediated enhanced Wnt/β-catenin signalling identified by the inventors is surprising for a number of reasons. Firstly, the interaction between Lgr proteins and Frizzled receptors and LRP proteins has not been previously described. Additionally, it is unexpected that Lgr, one of the Wnt target genes, should itself participate in Wnt signaling. It is also surprising that the Lgr-Frz-LRP receptor complex (termed herein the Wnt receptor complex) has two ligands, a feature only rarely found in nature.

Another surprising feature is that Lgr5 rarely occurs alone but is usually expressed in cells also expressing Lgr4. In contrast to this, both Lgr4 and Lgr6 are expressed exclusively in certain cells and tissues. This knowledge may be used to design compounds that enhance Wnt signaling in particular cells or tissues. For example, an Lgr6 agonist might specifically bind to epidermal cells, for which Lgr6 is an exclusive marker, and enhance Wnt signaling in these compartments e.g. to treat epithelial damage. By contrast, an Rspondin molecule, used to treat the same epithelial damage, would target all cells expressing Lgr4, Lgr5 or Lgr6 proteins and would be likely to cause a number of unwanted side-effects as a result. Conversely, these expression patterns also mean that treatments focusing solely on Lgr5 inhibition are likely to be redundant due to the presence and overlapping functionality of Lgr4 and/or Lgr6.

There are four secreted Rspondin proteins (Rspo1, Rspo2, Rspo3 and Rspo4), only encoded in vertebrate genomes. Loss-of-function mutations in Rspo1, Rspo3 and Rspo4 result in similar phenotypic defects to those seen in patients or mice lacking expression of Wnt ligands and/or receptors^{13, 14, 15}. Rspondin proteins do not bear sequence similarity to Wnt family proteins, but are often co-expressed with and induced by Wnt genes during embryogenesis. Rspondin proteins are particularly potent β-catenin activators when also present in the presence of secreted Wnt proteins.

Several theories have been suggested as to how Rspondin-1 might promote Wnt/β-catenin signalling: firstly, Rspondin-1 has been reported to be a high-affinity ligand of the Wnt co-receptor LRP6¹⁶; secondly, Rspo1 has been postulated to bind and block the Kremen protein that down-regulates surface expression of Wnt receptors¹⁷; thirdly, Rspo1 has been proposed to activate the Wnt pathway by blocking the interaction of the Wnt inhibitor Dickkopf1 (DKK1) with the LRP6 coreceptor¹⁸; and finally, identification of an interaction between Rspo1 and adult stem cell marker Lgr5 has led to the proposal of an alternative pathway for the activation of β-catenin signalling¹⁹.

However, despite these studies, the exact nature of the Rspondin receptors and the mechanism by which Rspondin drives canonical Wnt/β-catenin signals was unknown prior to the work by the inventors described herein. The work conducted by the present inventors provides the first realisation that the interaction between Rspondin and Lgr is part of a more complicated signalling mechanism requiring the presence of Frizzled Receptors and LRP proteins. The inventors are also the first to demonstrate that Rspondin proteins not only bind to Lgr4, Lgr5 and Lgr6 proteins but moreover they demonstrate the requirement of Lgr proteins for Rspondin-mediated activation of Wnt/β-catenin signalling, and thus show that Lgr proteins act as receptors for Rspondin that, in complex with Wnt, Frizzleds and LRPs, mediate enhanced canonical Wnt/β-catenin signalling. The authors have also demonstrated for the first time, that through binding to Lgr5, which is only expressed in stem cells, Rspondin plays a role in the activation and growth or stem cells.

Although blocking of binding of Rspondin proteins to Lgr5 by anti-Lgr5 antibodies was previously demonstrated and an alternative pathway for the activation of β-catenin signalling was hypothesised in WO2009/005809, the authors of this publication did not show effects on signalling by Rspondin-blocking Lgr5 antibodies, nor did they show any data of anti-Lgr5 antibodies in in vivo cancer models. No model for how Rspondin might mediate β-catenin signaling was proposed. Furthermore, given the latest data provided in the present invention showing that Lgr5 is only ever expressed in cells also expressing Lgr4, it is highly unlikely, that even if the authors of WO2009/005809 had tested their anti-Lgr5 antibody in in vivo cancer models, that they would have worked (i.e. treated the cancer and reduced the size of the tumour). This is because, although the antibody may have blocked Lgr5-mediated Wnt enhancement, Lgr4-mediated Wnt enhancement would be unaffected so the levels of Wnt activation would probably have remained at a similar level. The authors would not have known this at the time. Armed with the new knowledge about the Lgr4/Lgr5 expression patterns, as provided in the present application, it would be possible for a person skilled in the art to develop antagonists that block both Lgr4 and Lgr5 and thus much more effectively block Rspondin-mediated Wnt enhancement and provide a far more effective treatment to cancer and other diseases that are known to be treated by inhibition of Wnt signalling.

Furthermore, WO2009/005809 does not make the link between Lgr5 and stem cells. Therefore, the authors could not have known that Lgr5 was involved in normal stem cell growth. As such there is no suggestion to design agonists of Lgr5 (or of Lgr4 or Lgr6) as described herein. The skilled person would not be motivated by WO2009/005809 to design agonists of Lgr5 for stimulating stem cell growth.

The present inventors have also discovered that Lgr proteins are located on the basolateral side of epithelial cells. This knowledge makes possible the targeting of Rponding-mimicking agonists to the basolateral side of epithelial cells using other targets, for example tissue-specific targets, that are also known to be expressed basolaterally.

The identification of this surprising mechanism for Rspondin-induced Wnt/β-catenin signalling through the Lgr-Frizzled-LRP complex presents new opportunities for designing Rspondin mimics to activate Wnt/β-catenin signalling.

### Rspondin Proteins

The Rspondin protein that is mimicked by the agonist of the invention may be any known Rspondin protein since all of these Rspondin proteins are expected to fulfil the same role in the Wnt signalling pathway.

The Rspondin protein may thus be Rspondin 1 (gi: 284654; SEQ ID NO: 112), Rspondin 2 (gi: 340419; SEQ ID NO: 113); Rspondin 3 (gi: 84870; SEQ ID NO: 114) or Rspondin 4 (gi: 343637; SEQ ID NO: 115).

### Lgr Proteins

Lgr proteins are markers of adult stem cells²⁰. The agonist of the invention may mimic the effect of the Rspondin protein binding to any one of the Lgr family of proteins. In particular, the agonist of the invention may mimic the effect of the Rspondin protein binding to Lgr4 (gi: 55366), Lgr5 (gi: 8549) or Lgr6 (gi: 59352). Lgr4, Lgr5 and Lgr6 are all seven-transmembrane receptors that are close relatives of the receptors for the hormones FSH, LH and TSH.

Lgr5 is a Wnt target gene whose expression marks proliferative stem cells in multiple Wnt-dependent stem cell compartments, such as the small intestine and colon, the stomach, pancreas, liver, kidney, middle-ear, mammary gland, and the hair follicle. An agonist of the invention that mimics the effect of an Rspondin protein binding to Lgr5 would thus enhance canonical Wnt signals in all Lgr5⁺ cells, and hence increase proliferation in the stem cell compartment.

Lgr4 is much more widely expressed than Lgr5²¹. The inventors have previously shown that Lgr5 is co-expressed with Lgr4 in the stem-cell compartments mentioned above. Thus, Lgr5 marks intestinal stem cells at the base of crypts, while Lgr4 marks all crypt cells, including the Lgr5⁺ stem cells. An agonist of the invention that mimics the effect of an Rspondin protein binding to Lgr4 would enhance canonical Wnt signals in all Lgr4⁺ cells. Lgr4 is more widely expressed than Lgr5 and therefore, an agonist mimicking the effect of an Rspondin protein binding to Lgr4 of the invention would be expected to enhance Wnt signals and proliferation in a wider set of cells than a compound targeting Lgr5. For example, in the small intestine, all crypt cells express Lgr4 and therefore, an agonist of the invention that mimics the effect of an Rspondin protein binding to Lgr4 would enhance proliferation of all crypt cells.

Lgr6 marks multipotent stem cells in the epidermis²² and Lgr6 positive cells play a crucial role in wound healing and generation of smooth muscle tissues in many organs (e.g. airways, large arteries, uterus). An agonist of the invention that mimics the effect of an Rspondin protein binding to Lgr6 would enhance canonical Wnt signals in all Lgr6⁺ cells, and hence increase proliferation of these multipotent stem cells.

The inventors have discovered that the Lgr proteins are expressed on the basolateral side of epithelial cells. The basolateral membrane of a polarized cell is the surface of the plasma membrane that forms its basal and lateral surfaces. It faces towards the interstitium, and away from the lumen.

### Wnt Receptor Complex

As discussed above, the inventors have shown that Rspondin proteins facilitate Wnt-induced activation of the canonical Wnt pathway by binding to the Lgr protein when it is in a complex with one or more Frizzled (Frz) receptors and LRP proteins and optionally, other Lgr proteins. The agonist of the invention may therefore mimic the effect of the Rspondin protein binding to at least one Lgr protein in a complex with at least one Frz receptor and at least one LRP co-receptor. It was observed, for example, that a Wnt receptor complex can comprise at least two Lgr proteins (see FIG. 3). The Frz receptor in the complex is at least one of Frizzled 1 (Frz1; gi: 8321), Frizzled 2 (Frz2; gi: 2535), Frizzled 3 (Frz3; gi: 7976), Frizzled 4 (Frz4; gi: 8322), Frizzled 5 (Frz5; gi: 7855), Frizzled 6 (Frz6; gi: 8323), Frizzled 7 (Frz7; gi: 8324), Frizzled 8 (Frz8; gi: 8325), Frizzled 9 (Frz9; gi: 8326), and Frizzled 10 (Frz10; gi: 11211). In one example, the complex may comprise at least one of Frz5, Frz6 and Frz7. The LRP co-receptor in the complex may be any LRP protein family member. The complex must thus comprise at least one of LRP5 (gi: 4041) and/or LRP6 (gi: 4040).

The agonist of the invention may therefore mimic the effect of an Rspondin protein binding to an Lgr protein, wherein the Lgr protein, which may be Lgr4, Lgr5 or Lgr6, is in a complex with, for example, Frz5-LRP6, Frz6-LRP6, Frz7-LRP6 and other combinations of the proteins described above. In particular, the agonist of the invention may mimic the effect of an Rspondin protein binding to at least one Lgr protein, wherein the Lgr protein is Lgr4, Lgr5 and/or Lgr6 and is in a complex comprising Frz5, Frz6 and/or Frz7, and LRP5 and/or LRP6.

These complexes may further comprise a Wnt protein, such as WNT1 (BC074799; gi: 7471), WNT2 (BC029854; gi: 7472), WNT2b/13 (BC141825; gi: 7482), WNT3 (BC112118; gi: 7473), WNT3a (BC103921; gi: 89780), WNT4 (BC057781; gi: 54361), WNT5a (BC064694; gi: 7474), WNT5B (BC001749, gi: 81029), WNT6 (BC004329; gi: 7475), WNT7a (BC008811; gi: 7476), WNT7b (BC034923; gi: 7477), WNT8a (NM_{—}058244; gi: 7478), WNT8b (BC156632; gi: 7479), WNT9a (BC111960; gi: 7483), WNT9b (BC064534; gi: 7484), WNT10A (BC052234; gi: 80326), WNT10b (BC096353; gi: 7480), WNT11 (BC113388; gi: 7481), or WNT16 (BC104919; gi: 51384) (the first number in each bracket is a gene number as defined at: http://genome.ucsc.edu/cgi-bin/hgGateway). Preferentially, the complexes comprise Wnt5a or Wnt5B. The term “Wnt receptor complex” is used herein to refer to any such complex comprising at least one Lgr protein, an Frz receptor and an LRP protein, and optionally a Wnt protein.

### Functional Assays

The term “Rspondin-mimicking activity” refers to the ability of an agonist to mimic the effect or activity of an Rspondin protein binding to an Lgr protein. The ability of the agonists of the invention to mimic the activity of an Rspondin protein binding to an Lgr protein can be confirmed by a number of assays. The agonists of the invention typically initiate a reaction or activity that is similar to or the same as that initiated by the receptor's natural ligand Rspondin. In particular, the agonists of the invention enhance the canonical Wnt/β-catenin signalling pathway. As used herein, the term “enhances” refers to a measurable increase in the level of Wnt/β-catenin signalling compared with the level in the absence of Rspondin or in the absence of an agonist of the invention that mimics Rspondin.

Various methods are known in the art for measuring the level of canonical Wnt/β-catenin signalling. These include, but are not limited to assays that measure: Wnt/β-catenin target gene expression; TCF reporter gene expression; beta-catenin stabilization; LRP phosphorylation; Axin translocation from cytoplasm to cell membrane and binding to LRP.

The canonical Wnt/β-catenin signalling pathway ultimately leads to changes in gene expression through the transcription factors TCF7, TCF7L1, TCF7L2 and LEF. The transcriptional response to Wnt activation has been characterised in a number of cells and tissues^{23, 24, 25, 26, 27, 28, 29, 30}. As such, global transcriptional profiling by methods well known in the art can be used to assess Wnt/β-catenin signalling activation.

Changes in gene expression are generally mediated by TCF and LEF transcription factors. A TCF reporter assay assesses changes in the transcription of TCF/LEF controlled genes to determine the level of Wnt/β-catenin signalling. A TCF reporter assay was first described by Korinek, V. et al., 1997³¹. Also known as TOP/FOP this method involves the use of three copies of the optimal TCF motif CCTTTGATC, or three copies of the mutant motif CCTTTGGCC, upstream of a minimal c-Fos promoter driving luciferase expression (pTOPFLASH and pFOPFLASH, respectively) to determine the transactivational activity of endogenous β-catenin/TCF4. A higher ratio of these two reporter activities (TOP/FOP) indicates higher β-catenin/TCF4 activity.

Various other reporter transgenes that respond to Wnt signals exist intact in animals and therefore, effectively reflect endogenous Wnt signalling (reviewed in Barolo, 2006³²). These reporters are based on a multimerized TCF binding site, which drives expression of LacZ or GFP, which are readily detectable by methods known in the art. These reporter genes include: TOP-GAL³³ BAT-GAL³⁴ ins-TOPEGFP, ins-TOPGAL³⁵, LEF-EGFP³⁶ Axin2-LacZ³⁷, Axin2-d2EGFP³⁸, Lgr5tm1(cre/ERT2)³⁹, TOPdGFP⁴⁰.

The recruitment of dephosphorylated β-catenin to the membrane⁴¹, stabilisation and phosphorylation status of β-catenin⁴² and translocation of β-catenin to the nucleus (Klapholz-Brown Z et al., PLoS One. 2(9) e945, 2007)⁴³ in some cases mediated by complex formation with TCF transcription factors and TNIK^{23, 44} are key steps in the Wnt signalling pathway. Stabilisation is mediated by Disheveled family proteins that inhibit the “destruction” complex so that degradation of intracellular β-catenin is reduced, and translocation of β-catenin to the nucleus follows thereafter. Therefore, measuring the level and location of β-catenin in a cell is a good reflection of the level of Wnt/β-catenin signalling. A non-limiting example of such an assay is the “BioImage b-Catenin Redistribution Assay” (Thermo Scientific) which provides recombinant U2OS cells that stably express human β-catenin fused to the C-terminus of enhanced green fluorescent protein (EGFP). Imaging and analysis is performed with a fluorescence microscope or HCS platform allowing the levels and distribution of EGFP-β-catenin to be visualised.

Another way, in which the destruction complex is inhibited, is by removal of Axin by recruitment of Axin to the cytoplasmic tail of the Wnt co-receptor LRP^{45, 46}. Axin has been shown to bind preferentially to a phosphorylated form of the LRP tail⁴⁷. Visualisation of Axin translocation, for example with a GFP-Axin fusion protein, is therefore another method for assessing levels of Wnt/β-catenin signalling.

The agonists of the invention may enhance β-catenin signalling by at least 30%, 35%, 40%, 45%, 50%, 60%, 70%, 75%, 80%, 85%, 90%, 95%, 100%, 110%, 150%, 200%, 250%, 300%, 400% or 500% compared to the β-catenin signalling induced by a neutral substance or negative control as measured in an assay described above, for example as measured in the TOPFlash assay. A compound that does not bind to the Lgr protein, for example an irrelevant IgG1, can be used as a negative control in these assays. The agonists of the invention may enhance β-catenin signalling by a factor of 2×, 5×, 10×, 100×, 1000×, 10000× or more as compared to the activity in the absence of the agonist when measured in an assay described above, for example when measured in the TOPFlash assay, or any of the other assays mentioned herein.

Another method for testing whether a compound has Rspondin-mimicking activity, is to see whether it can rescue organoid growth in Rspondin-depleted culture medium. For example, using the stem cell medium described in Sato et al (Nature 459, 262-5, 2009), organoid growth can be tested with normal medium (comprising Rspondin), Rspondin-depleted medium, and a medium where Rspondin is replaced by the test compound. Rspondin is required for growth and proliferation of the organoids. In an Rspondin-depleted medium the cells do not form organoids. An agonist of the invention mimics the activity of Rspondin in the culture media and thus allows organoid growth.

In one embodiment, an agonist of the invention mimics Rspondin activity when used at about the same concentration as Rspondin (when used in an equivalent way). In another embodiment, an agonist of the invention mimics Rspondin activity when used at about 2×, 5×, 10×, 20×, 50× or 100× greater concentration than Rspondin. In another embodiment, an agonist of the invention mimics Rspondin activity when used at about 2×, 5×, 10×, 20×, 50× or 100× lower concentration than Rspondin.

In general, the agonists of the invention are free of any antagonistic activity. By antagonistic activity, it is meant that a compound prevents or reduces induction of any of the responses induced by Rspondin, including but are not limited to LRP phosphorylation, β-catenin stabilization, Axin translocation, increased expression of Wnt target genes measured in one of the assays described above. Antagonists may reduce induction of any one or more of these responses by 5%, 10%, 15%, 20%, 25%, 30%, 35%, preferably 40%, 45%, 50%, 55%, 60%, more preferably 70%, 80%, 85%, and most preferably 90%, 95%, 99%, or 100% compared to the responses detected in the presence of a neutral substance or negative control.

### Binding of the Agonist to the Lgr Protein

The agonist of the invention may act to mimic the activity of an Rspondin protein binding to an Lgr protein by binding to the Lgr protein, which may be Lgr4, Lgr5 or Lgr6. The agonist may bind to any region of the Lgr protein involved in activating the Lgr protein and enhancing Wnt/β-catenin signalling. A typical Lgr protein comprises the regions: N-terminal domain, leucine-rich repeats (LRRs), CRL-region (or hinge region), transmembrane (TM) regions, exodomains, intracellular domains and the C-terminal domain. These regions are described by the sequences laid out in table 1 below.

TABLE 1
SEQ ID NOs for the various regions of Lgr4, Lgr5 and Lgr6 polypeptides.

| SEQ | | | |
|---|---|---|---|
| ID | | | Protein |
| NO | Sequence | Polypeptide | Region |
| 1 | MPGPLGLLCFLALGLLGSAG | Lgr4 | Leader |
| 2 | PSGAAPPLCAAPCSCDGDRRVDCSGKGLTAVPEGLSAFTQA | Lgr4 | N-terminal |
| 3 | LDISMNNITQLPEDAFKNFPFLEE | Lgr4 | LRR1 |
| 4 | LQLAGNDLSFIHPKALSGLKELKV | Lgr4 | LRR2 |
| 5 | LTLQNNQLKTVPSEAIRGLSALQS | Lgr4 | LRR3 |
| 6 | LRLDANHITSVPEDSFEGLVQLRH | Lgr4 | LRR4 |
| 7 | LWLDDNSLTEVPVHPLSNLPTLQA | Lgr4 | LRR5 |
| 8 | LTLALNKISSIPDFAFTNLSSLVV | Lgr4 | LRR6 |
| 9 | LHLHNNKIRSLSQHCFDGLDNLETL | Lgr4 | LRR7 |
| 10 | DLNYNNLGEFPQAIKALPS | Lgr4 | LRR8 |
| 11 | LKELGFHSNSISVIPDGAFDGNPLLRT | Lgr4 | LRR9 |
| 12 | IHLYDNPLSFVGNSAFHNLSDLHS | Lgr4 | LRR10 |
| 13 | LVIRGASMVQQFPNLTGTVHLES | Lgr4 | LRR11 |
| 14 | LTLTGTKISSIPNNLCQEQKMLRT | Lgr4 | LRR12 |
| 15 | LDLSYNNIRDLPSFNGCHALEE | Lgr4 | LRR13 |
| 16 | ISLQRNQIYQIKEGTFQGLISLR | Lgr4 | LRR14 |
| 17 | ILDLSRNLIHEIHSRAFATLGPITN | Lgr4 | LRR15 |
| 18 | LDVSFNELTSFPTEGLNGLNQLK | Lgr4 | LRR16 |
| 19 | LVGNFKLKEALAAKDFVNLRSLSV | Lgr4 | LRR17 |
| 20 | PYAYQCCAFWGCDSYANLNTEDNSLQDHSVAQEKGTADAANV | Lgr4 | CRL |
| | TSTLENEEHSQIIIHCTPSTGAFKPCEYLLGSWMIR | | |
| 21 | LTVWFIFLVALFFNLLVILTTF | Lgr4 | TM1 |
| 22 | ASCTSLPSS | Lgr4 | intra-1 |
| 23 | KLFIGLISVSNLFMGIYTGILTFLDA | Lgr4 | TM2 |
| 24 | VSWGRFAEFGIWWETGSGCKV | Lgr4 | exo-1 |
| 25 | AGFLAVFSSESAIFLLMLATVER | Lgr4 | TM3 |
| 26 | SLSAKDIMKNGKSNHLKQ | Lgr4 | intra-2 |
| 27 | FRVAALLAFLGATVAGCFPLFHRGE | Lgr4 | TM4 |
| 28 | YSASPLCLPFPTGETPSL | Lgr4 | exo-2 |
| 29 | GFTVTLVLLNSLAFLLMAVIYTKLY | Lgr4 | TM5 |
| 30 | CNLEKEDLSENSQSSMIK | Lgr4 | intra-3 |
| 31 | HVAWLIFTNCIFFCPVAFFSFAPL | Lgr4 | TM6 |
| 32 | ITAISISPEIM | Lgr4 | exo-3 |
| 33 | KSVTLIFFPLPACLNPVLYVFFNP | Lgr4 | TM7 |
| 34 | KFKEDWKLLKRRVTKKSGSVSVSISSQGGCLEQDFYYDCGMYSHL | Lgr4 | C-terminal |
| | QGNLTVCDCCESFLLTKPVSCKHLIKSHSCPALAVASCQRPEGYW | | |
| | SDCGTQSAHSDYADEEDSFVSDSSDQVQACGRACFYQSRGFPLV | | |
| | RYAYNLPRVKD | | |
| 35 | PSGAAPPLCAAPCSCDGDRRVDCSGKGLTAVPEGLSAFTQALDIS | Lgr4 | N-terminal + |
| | MNNITQLPEDAFKNFPFLEE | | LRR1 |
| 36 | PSGAAPPLCAAPCSCDGDRRVDCSGKGLTAVPEGLSAFTQALDIS | Lgr4 | N-terminal + |
| | MNNITQLPEDAFKNFPFLEELQLAGNDLSFIHPKALSGLKELKV | | LRR1 + LRR2 |
| 37 | PSGAAPPLCAAPCSCDGDRRVDCSGKGLTAVPEGLSAFTQALDIS | Lgr4 | N-terminal + |
| | MNNITQLPEDAFKNFPFLEELQLAGNDLSFIHPKALSGLKELKVLT | | LRR1 + LRR2 + |
| | LQNNQLKTVPSEAIRGLSALQS | | LRR3 |
| 38 | MDTSRLGVLLSLPVLLQLAT | Lgr5 | Leader |
| 39 | GGSSPRSGVLLRGCPTHCHCEPDGRMLLRVDCSDLGLSELPSNLS | Lgr5 | N-terminal |
| | VFTSY | | |
| 40 | LDLSMNNISQLLPNPLPSLRFLEE | Lgr5 | LRR1 |
| 41 | LRLAGNALTYIPKGAFTGLYSLKV | Lgr5 | LRR2 |
| 42 | LMLQNNQLRHVPTEALQNLRSLQS | Lgr5 | LRR3 |
| 43 | LRLDANHISYVPPSCFSGLHSLRH | Lgr5 | LRR4 |
| 44 | LWLDDNALTEIPVQAFRSLSALQA | Lgr5 | LRR5 |
| 45 | MTLALNKIHHIPDYAFGNLSSLVV | Lgr5 | LRR6 |
| 46 | LHLHNNRIHSLGKKCFDGLHSLETL | Lgr5 | LRR7 |
| 47 | DLNYNNLDEFPTAIRTLSN | Lgr5 | LRR8 |
| 48 | LKELGFHSNNIRSIPEKAFVGNPSLIT | Lgr5 | LRR9 |
| 49 | IHFYDNPIQFVGRSAFQHLPELRT | Lgr5 | LRR10 |
| 50 | LTLNGASQITEFPDLTGTANLES | Lgr5 | LRR11 |
| 51 | LTLTGAQISSLPQTVCNQLPNLQV | Lgr5 | LRR12 |
| 52 | LDLSYNLLEDLPSFSVCQKLQK | Lgr5 | LRR13 |
| 53 | IDLRHNEIYEIKVDTFQQLLSLR | Lgr5 | LRR14 |
| 54 | SLNLAWNKIAIIHPNAFSTLPSLIK | Lgr5 | LRR15 |
| 55 | LDLSSNLLSSFPITGLHGLTHLK | Lgr5 | LRR16 |
| 56 | LTGNHALQSLISSENFPELKVIEM | Lgr5 | LRR17 |
| 57 | PYAYQCCAFGVCENAYKISNQWNKGDNSSMDDLHKKDAGMF | Lgr5 | CRL |
| | QAQDERDLEDFLLDFEEDLKALHSVQCSPSPGPFKPCEHLLDGWL | | |
| | IR | | |
| 58 | IGVWTIAVLALTCNALVTSTVF | Lgr5 | TM1 |
| 59 | RSPLYISPI | Lgr5 | intra-1 |
| 60 | KLLIGVIAAVNMLTGVSSAVLAGVDA | Lgr5 | TM2 |
| 61 | FTFGSFARHGAWWENGVGCHV | Lgr5 | exo-1 |
| 62 | IGFLSIFASESSVFLLTLAALER | Lgr5 | TM3 |
| 63 | GFSVKYSAKFETKAPFSS | Lgr5 | intra-2 |
| 64 | LKVIILLCALLALTMAAVPLLGGSK | Lgr5 | TM4 |
| 65 | YGASPLCLPLPFGEPSTM | Lgr5 | exo-2 |
| 66 | GYMVALILLNSLCFLMMTIAYTKLY | Lgr5 | TM5 |
| 67 | CN LDKGDLENIWDCSMVK | Lgr5 | intra-3 |
| 68 | HIALLLFTNCILNCPVAFLSFSSL | Lgr5 | TM6 |
| 69 | INLTFISPEVI | Lgr5 | exo-3 |
| 70 | KFILLVVVPLPACLNPLLYILFNP | Lgr5 | TM7 |
| 71 | HFKEDLVSLRKQTYVWTRSKHPSLMSINSDDVEKQSCDSTQALV | Lgr5 | C-terminal |
| | TFTSSSITYDLPPSSVPSPAYPVTESCHLSSVAFVPCL | | |
| 72 | GGSSPRSGVLLRGCPTHCHCEPDGRMLLRVDCSDLGLSELPSNLS | Lgr5 | N-terminal + |
| | VFTSYLDLSMNNISQLLPNPLPSLRFLEE | | LRR1 |
| 73 | GGSSPRSGVLLRGCPTHCHCEPDGRMLLRVDCSDLGLSELPSNLS | | |
| | VFTSYLDLSMNNISQLLPNPLPSLRFLEELRLAGNALTYIPKGAFTG | Lgr5 | N-terminal + |
| | LYSLKV | | LRR1 + LRR2 |
| 74 | GGSSPRSGVLLRGCPTHCHCEPDGRMLLRVDCSDLGLSELPSNLS | Lgr5 | N-terminal + |
| | VFTSYLDLSMNNISQLLPNPLPSLRFLEELRLAGNALTYIPKGAFTG | | LRR1 + LRR2 + |
| | LYSLKVLMLQNNQLRHVPTEALQNLRSLQS | | LRR3 |
| 75 | MPSPPGLRALWLCAALCASRRAGG | Lgr6 | Leader |
| 76 | APQPGPGPTACPAPCHCQEDGIMLSADCSELGLSAVPGDLDPLT | Lgr6 | N-terminal |
| | AY | | |
| 77 | LDLSMNNLTELQPGLFHHLRFLEE | Lgr6 | LRR1 |
| 78 | LRLSGNHLSHIPGQAFSGLYSLKI | Lgr6 | LRR2 |
| 79 | LM LQNNQLGGIPAEALWELPSLQS | Lgr6 | LRR3 |
| 80 | LRLDANLISLVPERSFEGLSSLRH | Lgr6 | LRR4 |
| 81 | LWLDDNALTEIPVRALNNLPALQA | Lgr6 | LRR5 |
| 82 | MTLALNRISHIPDYAFQNLTSLVV | Lgr6 | LRR6 |
| 83 | LHLHNNRIQHLGTHSFEGLHNLETL | Lgr6 | LRR7 |
| 84 | DLNYNKLQEFPVAIRTLGR | Lgr6 | LRR8 |
| 85 | LQELGFHNNNIKAIPEKAFMGNPLLQT | Lgr6 | LRR9 |
| 86 | IHFYDNPIQFVGRSAFQYLPKLHT | Lgr6 | LRR10 |
| 87 | LSLNGAMDIQEFPDLKGTTSLEI | Lgr6 | LRR11 |
| 88 | LTLTRAGIRLLPSGMCQQLPRLRVLELSHN | Lgr6 | LRR12 |
| 89 | QIEELPSLHRCQKLEEIGLQHNRI | Lgr6 | LRR13 |
| 90 | WEIGADTFSQLSSLQALDLSWN | Lgr6 | LRR14 |
| 91 | AIRSIHPEAFSTLHSLVKLDLTD | Lgr6 | LRR15 |
| 92 | NQLTTLPLAGLGGLMHLK | Lgr6 | LRR16 |
| 93 | LKGNLALSQAFSKDSFPKLRILEV | Lgr6 | LRR17 |
| 94 | PYAYQCCPYGMCASFFKASGQWEAEDLHLDDEESSKRPLGLLAR | Lgr6 | CRL |
| | QAENHYDQDLDELQLEMEDSKPHPSVQCSPTPGPFKPCEYLFES | | |
| | WIG | | |
| 95 | RLAVWAIVLLSVLCNGLVLLTVF | Lgr6 | TM1 |
| 96 | AGGPVPLPPV | Lgr6 | intra-1 |
| 97 | KFVVGAIAGANTLTGISCGLLASVDAL | Lgr6 | TM2 |
| 98 | TFGQFSEYGARWETGLGCRAT | Lgr6 | exo-1 |
| 99 | GFLAVLGSEASVLLLTLAAVQC | Lgr6 | TM3 |
| 100 | SVSVSCVRAYGKSPSLGS | Lgr6 | intra-2 |
| 101 | VRAGVLGCLALAGLAAALPLASVGE | Lgr6 | TM4 |
| 102 | YGASPLCLPYAPPEGQPAAL | Lgr6 | exo-2 |
| 103 | GFTVALVM M NSFCFLVVAGAYIKLY | Lgr6 | TM5 |
| 104 | CDLPRGDFEAVWDCAMVRH | Lgr6 | intra-3 |
| 105 | VAWLIFADGLLYCPVAFLSFASM | Lgr6 | TM6 |
| 106 | LGLFPVTPEAV | Lgr6 | exo-3 |
| 107 | KSVLLVVLPLPACLNPLLYLLFNPH | Lgr6 | TM7 |
| 108 | FRDDLRRLRPRAGDSGPLAYAAAGELEKSSCDSTQALVAFSDVDL | Lgr6 | C-terminal |
| | ILEASEAGRPPGLETYGFPSVTLISCQQPGAPRLEGSHCVEPEGNH | | |
| | FGNPQPSMDGELLLRAEGSTPAGGGLSGGGGFQPSGLALLHTYE | | |
| | FCRYPAQWRPLESRGPV | | |
| 109 | APQPGPGPTACPAPCHCQEDGIMLSADCSELGLSAVPGDLDPLT | Lgr6 | N-terminal + |
| | AYLDLSMNNLTELQPGLFHHLRFLEE | | LRR1 |
| 110 | APQPGPGPTACPAPCHCQEDGIMLSADCSELGLSAVPGDLDPLT | Lgr6 | N-terminal + |
| | AYLDLSMNNLTELQPGLFHHLRFLEELRLSGNHLSHIPGQAFSGLY | | LRR1 + LRR2 |
| | SLKI | | |
| 111 | APQPGPGPTACPAPCHCQEDGIMLSADCSELGLSAVPGDLDPLT | Lgr6 | N-terminal + |
| | AYLDLSMNNLTELQPGLFHHLRFLEELRLSGNHLSHIPGQAFSGLY | | LRR1 + LRR2 + |
| | SLKILMLQNNQLGGIPAEALWELPSLQS | | LRR3 |

In one embodiment, the agonist binds to the extracellular parts or transmembrane regions of the Lgr protein. In a further embodiment, the agonist binds to the extracellular parts of the Lgr protein comprising the N-terminal region, any of the 17 leucine rich repeats, the CRL region and/or any of the 3 exodomains (as designated exo-1, exo-2 and exo-3 in table 1).

In one embodiment, the agonist may bind to one or more of the leucine-rich repeats (LRRs) of the Lgr protein. For example, the agonist may bind a region consisting of or comprising LRR1, LRR2, LRR3, LRR4, LRR5, LRR6, LRR7, LRR8, LRR9, LRR10, LRR11, LRR12, LRR13, LRR14, LRR15, LRR16, and/or LRR17 of any of the Lgr4, Lgr5 or Lgr6 polypeptides, as shown in Table 1 above, or to an epitope within these regions. Alternatively, the agonist may bind to epitopes within a region consisting of or comprising the N-terminus plus LRR1-3, or N-terminus plus LRR1 and LRR2, or N-terminus plus LRR1 of any of the Lgr4, Lgr5 or Lgr6 polypeptides, as shown in Table 1 above. As explained below, the inventors have shown that Rspondin binds to the LRRs of Lgr proteins and therefore, agonists that mimic Rspondin activity may also bind to this region. Alternatively, the agonist may bind to a conformational epitope that may be induced upon recruitment of the Lgr protein to the Wnt/Frz/LRP complex.

In an alternative embodiment, the agonist of the invention may bind to the extracellular domain or external transmembrane loops of the Lgr protein or to an epitope within these regions. The Lgr protein is a 7 transmembrane domain protein and the agonist may bind to a region of the Lgr protein that is extracellular in the native state of the protein, or to an epitope within these regions. For example, in one embodiment, the agonist may bind to an epitope within the CRL-region of any of the Lgr4, Lgr5 or Lgr6 polypeptides, as shown in Table 1 above. Another possibility is that the agonist of the invention may bind to any one of the three exodomains or combinations thereof that are located between the transmembrane regions of the Lgr peptide. For example, the agonist of the invention may bind to an epitope within a region of the protein consisting of or comprising exo-1, exo-2, and/or exo-3 of any of the Lgr4, Lgr5 or Lgr6 polypeptides, as shown in Table 1 above.

The inventors have shown that antibody 1D9 mimics Rspondin activity and is therefore an agonist (see Example 3). FIG. 26 shows that antibody 1D9 does not bind to Lgr5-478, which represents an Lgr5 fragment comprising the exo domain of Lgr5 without the CRL region. However, antibody 1D9 does bind to Lgr5-543, which represents the full exo domain of Lgr5 (the CRL and the LRRs). Therefore, the inventors conclude that antibody 1D9 binds to the CRL region of Lgr5. SEQ ID NO: 57 represents the CRL region (or hinge region) of Lgr5. The inventors have thus identified a region on Lgr5 which is involved in the activation of Wnt signalling by Lgr5. This permits the design and screening of agonists that target a region of Lgr5 comprising or consisting of the whole or part of the CRL region i.e. comprising or consisting of the whole or part of SEQ ID NO:57 (see section on “Methods for identifying further agonists of the invention”).

Therefore, in one embodiment, an agonist of the invention binds to a region consisting of or comprising the CRL (or hinge region) of Lgr4, Lgr5 and/or Lgr6, or i.e. the region represented by SEQ ID NOs: 20, 57 and/or 94 respectively. In some embodiments an agonist of the invention binds to the region of Lgr5 represented by SEQ ID NO: 57 but does not bind to the region of Lgr4 represented by SEQ ID NO: 20 or the region of Lgr6 represented by SEQ ID NO: 94. In some embodiments an agonist of the invention binds to the region of Lgr4 represented by SEQ ID NO: 20 but does not bind to the region of Lgr5 represented by SEQ ID NO: 57 or the region of Lgr6 represented by SEQ ID NO: 94. In some embodiments an agonist of the invention binds to the region of Lgr6 represented by SEQ ID NO: 94 but does not bind to the region of Lgr4 represented by SEQ ID NO: 20 or the region of Lgr5 represented by SEQ ID NO: 57.

In some embodiments the agonist is an antibody that does not bind the CRL region of Lgr4, Lgr5 and/or Lgr6 i.e. the region represented by SEQ ID NOs: 20, 57 and/or 94 respectively.

The inventors have also demonstrated that antibody 1D9 and Rspondin bind to separate regions on Lgr5. FIG. 26 demonstrates that both antibody 1D9 and Rspondin can bind to Lgr5-543 (full exo domain of Lgr5 i.e. CRL and LRRs) at the same time to form a complex. FIG. 26 also shows that Rspondin binds to Lgr5-478 which represents the exo domain without the CRL region. Therefore, the inventors conclude that Rspondin binds to the LRRs on Lgr proteins. It is surprising that agonistic antibody 1D9 binds to a region on Lgr5 that differs from the region that Rspondin binds to.

Another possibility is that the agonist of the invention may bind to one or more transmembrane region of the Lgr peptide. For example, the agonist of the invention may bind to a region of the protein consisting of or comprising TM1, TM2, TM3, TM4, TM5, TM6, and/or TM7 of any of the Lgr4, Lgr5 or Lgr6 polypeptides, as shown in Table 1 above, or to an epitope within these regions. For example, in one embodiment, the agonist may be a small hydrophobic molecule that binds in the transmembrane region and disrupts protein-lipid interactions, causing favourable conformational or biochemical changes which result in agonistic activity.

In a further embodiment, the agonist may be a competitive agonist that binds to the Rspondin binding site in the Lgr protein. The Rspondin binding site is typically located at the N-terminus of the Lgr protein. The N-terminus, leucine-rich repeat 1 and leucine-rich repeat 2 (this region is described by SEQ ID NOs: 36, 73, 110 for Lgr4, Lgr5 and Lgr6 respectively) are essential for binding of Rspondin1-4 to of any of the Lgr4, Lgr5 or Lgr6 polypeptides. It is predicted that this holds true for all three Lgrs and all four Rspondins based on sequence homology. The agonist may bind to an epitope within an Rspondin binding site on an Lgr protein, in particular to at least 1, 2, 3, 4, 5, 6 or more amino acids within the Rspondin binding site.

Such competitive agonists can be identified by standard methods known in the art. For example, the skilled person might identify agonists that compete for Rspondin binding in a competitive ELISA assay. Preferred competitive agonists bind to the Lgr protein with a greater affinity and/or avidity than that of Rspondin. Assays for identifying compounds that bind to the Lgr proteins are described in more detail below in connection with methods of screening for agonists of the invention.

An epitope is the site on the antigen to which a binding agent, such as for example a ligand or an antibody, binds. If the antigen is a polymer, such as a protein or polysaccharide, the epitope can be formed by contiguous residues or by non-contiguous residues brought into close proximity by the folding of an antigenic polymer. In proteins, epitopes formed by contiguous amino acids are typically retained on exposure to denaturing solvents, whereas epitopes formed by noncontiguous amino acids are typically lost under said exposure.

The agonist of the invention may bind to any epitope within these regions of the Lgr proteins, in particular to any amino acid sequence of at least 1, 2, 3, 4, 5, 6 or more amino acids within these regions. The agonists may bind to epitopes within one or more of these regions, either simultaneously or separately. By simultaneously, it is meant that the compound of the invention can bind to epitopes within two or more regions of the Lgr protein at the same time. By separately, it is meant that the compound can only be bound to one epitope within an Lgr region at a single moment in time, but also has capacity to bind an epitope in another region at a different time.

The ability of an agonist of the invention to bind to an Lgr protein can be determined by standard experimental techniques known in the art including immunoprecipitation, western blot, biacore (IBIS), FACS, and mass spectrometry.

The agonist of the invention typically binds to the Lgr protein with a Kd equal to or less than about 10⁻⁷M, 10⁻⁸M, 10⁻⁹M, 10⁻¹⁰M, 10⁻¹¹M, 10⁻¹²M, 10⁻¹³M or more. Such values can be determined by binding assays, for example, by surface plasmon resonance or the Kinexa method, as practised by those of skill in the art.

In one aspect of the invention, the agonist binds specifically to the Lgr protein. The term “specific” means that the compound has substantially greater affinity for Lgr polypeptides than their affinity for other related polypeptides. For example, a compound that is specific for Lgr4 will have substantially greater affinity for Lgr4 than for Lgr5 or Lgr6. By “substantially greater affinity” it is meant that there is a measurable increase in the affinity for an Lgr protein as compared with the affinity for other known receptor proteins. In one embodiment, an agonist of the invention is specific for Lgr4, Lgr5 and Lgr6. This means that it has substantially greater affinity for all of these Lgr polypeptides than for other related polypeptides. In another embodiment, an agonist of the invention is specific for only one or two of Lgr4, Lgr5 and/or Lgr6. This means that it has substantially greater affinity for one or two Lgr polypeptides than for another Lgr polypeptide. Therefore, an agonist of the invention that is specific for Lgr5, need not bind and activate only Lgr5. However, in some embodiments, the agonist of the invention that is specific for Lgr5 preferentially binds and/or activates Lgr5 more than Lgr4 or Lgr6. For example, an agonist may preferentially bind to Lgr5 compared to Lgr4 and Lgr6. Alternatively, it may bind to Lgr4, Lgr5 and Lgr6 with equal affinity but preferentially activate Lgr5 more than the others. The same definition applies for an agonist of the invention that is specific for Lgr4 or Lgr6. In other embodiments, an agonists may bind to and activate only a single class of Lgr molecule i.e. only to Lgr4, Lgr5 or Lgr6.

### Exemplary Agonists

### Small Molecules

Agonists of the invention, in particular agonists that bind to Lgr proteins according to the invention may exist in various forms, including natural or modified substrates, enzymes, receptors, small organic molecules, such as small natural or synthetic organic molecules of up to 2000 Da, preferably 800 Da or less, peptidomimetics, inorganic molecules, peptides, polypeptides, aptamers, and structural or functional mimetics of these including small molecules.

In some embodiments of the invention, the agonist is a modified substrate. For example the modified substrate may comprise or consist of an Rspondin fragment, mutant or derivative that binds Lgr4, Lgr5 and/or Lgr6.

Therefore, in some embodiments, the agonist of the invention is a fragment of Rspondin. Fragments of Rspondin might have advantages over full-length Rspondin. For example, smaller protein fragments are easier to generate by recombinant methods and are more likely to be more stable because they have fewer enzyme cutting sites. Fragments are also more likely to be lacking regulatory domains that may make dosing unpredictable. The use of an Rspondin fragment may be preferable over a full length Rspondin protein, because smaller fragments have enhanced stability in serum, and because fragments generated by recombinant techniques are easier than full length Rspondin proteins to produce in E. coli.

In some embodiments of the invention, an Rspondin fragment comprises at least 50, at least 100, at least 150 or at least 200 consecutive amino acids of SEQ ID NOs: 112, 113, 114 or 115 or of sequences with more than 70, 80, 90 or 99% identity to any one of SEQ ID NOs: 112, 113, 114 or 115. An Rspondin fragment is by definition at least one amino acid shorter than full length wild-type Rspondin. Therefore, an Rspondin fragment is less than 263 amino acids of SEQ ID NO: 112, less than 243 amino acids of SEQ ID NO: 113, less than 272 amino acids of SEQ ID NO: 114, or less than 234 amino acids of SEQ ID NO: 115. In some embodiments the Rspondin fragment comprises or consists of less than 220, less than 200, less than 180, less than 160, less than 140, less than 120, less than 100, less than 80, less than 70, less than 60, less than 50, less than 40, less than 30, less than 20 consecutive amino acids of SEQ ID NOs: 112, 113, 114 or 115 or of sequences with more than 70, 80, 90 or 99% identity to any one of SEQ ID NOs: 112, 113, 114 or 115.

Full length, wild-type Rspondin proteins comprise a Furin domain, a Thrombospondin domain and a region of basic amino acid-rich repeats (Dev Cell 2004, 7, 525-534, Kazanskaya et al). The Furin domain consists of SEQ ID NOs: 116, 117, 118 and 119 in Rspondin 1, 2, 3 and 4 respectively. The Thrombospondin domain consists of SEQ ID NOs: 120, 121, 122 and 123 in Rspondin 1, 2, 3 and 4 respectively. The region of basic amino acid-rich repeats consists of SEQ ID NOs: 124, 125, 126 and 127 in Rspondin 1, 2, 3 and 4 respectively. The inventors have discovered for the first time that truncated Rspondin proteins without the basic amino acid-rich repeats (SEQ ID NO: 139) and truncated Rspondin proteins without the basic amino acid-rich repeats and without the thrombospondin domain (SEQ ID NO: 140), can still bind to Lgr proteins (for example, see Example 2). Furthermore, the inventors have shown that the “Furin domain fragments” (lacking Thromospondin and the amino acid-rich repeats and represented by SEQ ID NO: 140) are more potent at enhancing the Wnt pathway than full-length Rspondin at the same concentration (see FIG. 22).

Thus, in some embodiments, the Rspondin fragment does not comprise at least part of the Thrombospondin domain and/or at least part of the regions of basic amino acid-rich repeats. In some embodiments, the Rspondin fragment does not comprise the Thrombospondin domain and/or the region of basic amino acid-rich repeats.

In some embodiments, the Rspondin fragment is the fragment represented by SEQ ID NO: 139 or SEQ ID NO: 140.

Furthermore, the inventors have shown that an anti-Rspondin 3 monoclonal antibody, which binds to epitopes in the Furin domain of Rspondin 3, blocks the Wnt-enhancing activity of Rspondin (for example, see FIG. 21). Therefore, the inventors conclude that the Furin domain is sufficient and necessary for binding and for the activity of Rspondin. The skilled person could not have expected that the Furin domain alone would be necessary for Rspondin binding to Lgr proteins and sufficient for mimicking the Wnt-enhancing activity of Rspondin. In one embodiment of the invention, the Rspondin fragment comprises or consists of an Rspondin Furin domain, for example selected from SEQ ID NOs: 116, 117, 118, 119, 140, or 143, and an N-terminal domain. In a further embodiment of the invention, the Rspondin fragment comprises or consists of an Rspondin Furin domain. The Rspondin Furin domain may have more than 70, 80, 90 or 99% identity to SEQ ID NOs: 116, 117, 118, 119, 140, or 143. The Rspondin Furin domain may consist of SEQ ID NOs: 116, 117, 118, 119, 140, or 143. Alternatively, the Rspondin fragment comprises at least 50, at least 60, at least 70, at least 80 or at least 90 consecutive amino acids of SEQ ID NOs: 116, 117, 118, 119, 140 or 143. In some embodiments the Rspondin Furin domain fragment comprises or consists of less than 100, less than 90, less than 80, less than 70, less than 60, less than 50, less than 40, less than 30, less than 20, less than 10 consecutive amino acids of SEQ ID NOs: 116, 117, 118, 119, 140, or 143 or of sequences with more than 70, 80, 90 or 99% identity to any one of SEQ ID NOs: 116, 117, 118, 119, 140, or 143.

Sequence identity between polypeptide sequences is preferably determined by pairwise alignment algorithm using the Needleman-Wunsch global alignment algorithm (Needleman & Wunsch (1970) J. Mol. Biol. 48, 443-453), using default parameters (e.g. with Gap opening penalty=10.0, and with Gap extension penalty=0.5, using the EBLOSUM62 scoring matrix). This algorithm is conveniently implemented in the needle tool in the EMBOSS package (Rice et al. (2000) Trends Genet 16:276-277). Sequence identity should be calculated over the entire length of the polypeptide sequence of the invention.

In a further embodiment of the invention, the Rspondin fragment that comprises or consists of an Rspondin Furin domain, additionally comprises of an Rspondin Thrombospondin domain, for example as shown in SEQ ID NO: 139.

In some embodiments of the invention, the agonist may comprise more than one Rspondin fragment from either the same or one or more different Rspondin proteins. These fragments may be joined together in any order e.g. by genetic recombination or by a linking molecule. In a preferred embodiment, the fragments may be joined in the same order that they might appear in a full length wild-type Rspondin protein. For example, the agonist may comprise the Furin domain of Rspondin 1 and the Thrombospondin domain of Rspondin 2, 3 or 4, or vice versa. The skilled person will appreciate that many combinations of Rspondin 1, 2, 3 and 4 fragments are possible and that many combinations would be expected to result in fully functional Rspondin proteins.

In some embodiments, the agonist is a mutant Rspondin or Rspondin derivative that is specific to only Lgr4, Lgr5 or Lgr6. For example in some embodiments the agonist is an Rspondin derivative specific to only Lgr5, for example by mutation of one or more amino acid residues involved in binding to the Lgr4, Lgr5 and Lgr6 proteins.

In some embodiments of the invention, an Rspondin derivative may be, for example, Rspondin1, Rspondin2, Rspondin3 or Rspondin4 with one or more amino acid mutations, including substitutions, insertions or deletions, that cause it to have increased specificity for one class of Lgr molecule compared to another. Identification of such amino acid substitutions, insertions or deletions could be determined by protein engineering techniques known in the art. For example, an Rspondin derivative may be engineered to have increased specificity for Lgr5 compared to Lgr4 or Lgr6. Alternatively, an Rspondin derivative may bind and activate only Lgr5 and not Lgr4 or Lgr6. In an alternative embodiment, the Rspondin derivative is specific for, or has greater specificity for Lgr4 (relative to Lgr5 and/or Lgr6) or Lgr6 (relative to Lgr4 and/or Lgr5). This would be advantageous for use in therapy, for example, because wild-type Rspondin binds to Lgr4, Lgr5 and Lgr6 and thus stimulates growth, via the Wnt pathway, in all proliferating cells. An Rspondin derivative that has increased specificity for Lgr5, would preferentially stimulate growth of Lgr5-positive stem cells, not in all dividing cells. Therefore, in situations where wild-type Rspondin causes too much regrowth, an Rpsondin derivative with specificity for only a single Lgr protein, for example Lgr5 or Lgr6, would be more appropriate for regenerative treatments.

An example of a variant of Rspo1 is shown in SEQ ID NO: 128, and has a single mutation V50I. In one embodiment of the invention the Rspondin derivative is not the variant of Rspo1 that is shown in SEQ ID NO: 128.

In some embodiments, the agonist of the invention is not an Rspondin protein. In some embodiments, the agonist of the invention is not a fragment of an Rspondin protein. In some embodiments, the agonist of the invention is not a derivative of Rspondin. In some embodiments, the agonist of the invention is not a furin domain fragment. This is particularly envisaged in some embodiments relating to the agonist per se. However, in other aspects of the invention, it is envisaged that an Rspondin protein, fragment or derivative, for example a furin domain fragment is encompassed by the invention, for example wherein the agonist is a multi-targeting compound and/or when the agonist or multi-targeting compound is present/used in for example pharmaceutical compositions, multi-targeting compounds, methods for treatment, methods for enhancing cell growth/proliferation and methods for identifying new agonists.

In one embodiment of the invention, the agonist is an aptamer. As used herein, the term “aptamer” refers to strands of oligonucleotides (DNA or RNA) that can adopt highly specific three-dimensional conformations. Aptamers are designed to have high binding affinities and specificities towards certain target molecules, including extracellular and intracellular proteins.

### Antibodies

In another embodiment, the agonist is an antibody.

A conventional antibody is comprised of two identical heavy chains and two identical light chains that are joined by disulfide bonds. Each heavy and light chain contains a constant region and a variable region. Each variable region contains three CDRs which are primarily responsible for binding an epitope of an antigen, in this case the Lgr protein, as discussed above. They are referred to as CDR1, CDR2, and CDR3, numbered sequentially from the N-terminus, of which the CDR3 region comprises the most variable region and normally provides a substantial part of the contact residues to a target. The more highly conserved portions of the variable regions are called the “framework regions”.

The term antibody is used herein in the broadest sense and specifically covers, but is not limited to, monoclonal antibodies (including full length monoclonal antibodies) of any isotype such as IgG, IgM, IgA, IgD and IgE, polyclonal antibodies including recombinant polyclonal antibodies, Oligoclonics, multispecific antibodies, chimeric antibodies, nanobodies, diabodies, BiTE's, Tandabs, mimetobodies, bispecific antibodies, humanized antibodies, human antibodies, deimmunised antibodies and antibody fragments. In addition, scaffolds will be covered under this term, such as Anticalins, Ankarins, etc. An antibody reactive with a the specific epitopes of the Lgr proteins discussed above can be generated by recombinant methods such as selection of libraries of recombinant antibodies in phage or similar vectors, or by immunizing an animal with the Lgr epitopes of nucleic acid encoding them.

Antibodies of the invention will have binding properties described above for agonists in general. Antibody characteristics, such as on-rates (ka), off-rates (kd) and affinities (KD) can be determined in competitive binding assays using known platforms such as Octet™ (ForteBio), ProteOn™ (Bio-Rad), and Biacore™ (GE Healthcare). A population of monoclonal antibodies consists of antibodies directed against a single epitope. The monoclonal antibodies are essentially identical, in general because they are made by identical immune cells that are all clones of a particular parent cell. There is a possibility that naturally occurring mutations may cause differences between monoclonal antibodies in the same population in minor amounts. Monoclonal antibodies to Lgr proteins that mimic the activity of Rspondin by binding to the Lgr protein, e.g. by binding the agonistic epitopes discussed above, can also be readily produced by one skilled in the art. The general methodology for making monoclonal antibodies using hybridoma technology is well known. In addition antibodies of this type can be generated by phage display technology or ribosome display technology. Panels of monoclonal antibodies produced against the Lgr proteins can be screened to confirm that they are agonists, as well as for various properties, such as isotype, epitope, affinity, etc.

In one embodiment, an antibody according to the invention comprises a single domain antibody, a F(ab′)2, Fab, Fab′, Facb, or single chain Fv (scFv) fragment. An Fc fragment, which for example activates complement and may bind to Fc receptors, can be present but is not required for an antibody and variants or derivatives thereof. A scFv fragment is an epitope-binding fragment that contains at least one fragment of an antibody heavy chain variable region (VH) linked to at least one fragment of an antibody light chain variable region (VL). The linker may be a short, flexible peptide selected to assure that the proper three-dimensional folding of the VL and VH regions occurs once they are linked so as to maintain the target molecule binding-specificity of the whole antibody from which the single-chain antibody fragment is derived. The carboxyl terminus of the VL or VH sequence may be covalently linked by a linker to the amino acid terminus of a complementary VL or VH sequence.

The antibody may be a nanobody, and/or a bispecific antibody. A nanobody is a single domain antibody that occurs naturally in camelids. In contrast to standard antibodies, nanobodies are relatively simple proteins comprising only a heavy chain-like variable region. Bispecific antibodies are artificially engineered monoclonal antibodies that consist of two distinct binding sites and are capable of binding two different epitopes. Examples of bispecific antibodies are discussed in more detail below in the section on dual-targeting and multi-targeting agonists.

The antibody may be a chimeric antibody comprising a binding portion, for example the variable region or part thereof of the heavy and light chains, of a non-human antibody, while the remainder portion, for example the constant region of the heavy and light chains, is of a human antibody. A chimeric antibody may be produced by recombinant processes well known in the art, and has an animal variable region and a human constant region.

The antibody may be a human antibody. The term “human antibody” means an antibody in which the variable and constant domain sequences are derived from human sequences. A human antibody can be made by several different ways, including by use of human immunoglobulin expression libraries (Stratagene Corp., La Jolla, Calif.) to produce fragments of human antibodies VH, VL, Fv, Fd, Fab, or (Fab′)2, and using these fragments to construct whole human antibodies using techniques similar to those for producing chimeric antibodies. Alternatively, these fragments may be used on their own. Human antibodies can also be produced in transgenic mice with a human immunoglobulin genome. Such mice are available from Abgenix. Inc., Fremont, Calif., Kyowa Hakko Kirin (KM mouse), Ablexis (AlivaMab mouse) and Medarex, Inc., Annandale, N.J. Hybridomas can be generated by conventional procedures by fusing B lymphocytes from the immunized animals with myeloma cells (e.g., Sp2/0 and NSO), as described by Kohler, G. and Milstein, C. 1975⁴⁸.

In a humanized antibody, only the complementarity determining regions (CDRs), which are responsible for antigen binding and specificity are animal derived and have an amino acid sequence corresponding to the animal antibody, and substantially all of the remaining portions of the molecule (except, in some cases, small portions of the framework regions within the variable region) are human derived and correspond in amino acid sequence to a human antibody^{49, 50}. Methods for humanizing non-human antibodies are known in the art. As is known to the skilled person, antibodies such as rat antibodies can be humanized by grafting their CDRs onto the variable light (VL) and variable heavy (VH) frameworks of human Ig molecules, while retaining those rat framework residues deemed essential for specificity and affinity⁵¹. Overall, CDR grafted antibodies consist of more than 80% human amino acid sequences^{52, 53}. Despite these efforts, CDR-grafted, humanized antibodies were shown to still evoke an antibody response against the grafted V region⁵⁴.

A deimmunised antibody is an antibody in which the T and B cell epitopes have been eliminated. They have reduced immunogenicity when applied in vivo. To further decrease the content of non-human sequences in therapeutic mAbs, humanization methods based on different paradigms such as resurfacing⁵⁵, superhumanization⁵⁶, human string content optimization⁵⁷ and humaneering have been developed⁵⁸. As in CDR grafting approaches, these methods rely on analyses of the antibody structure and sequence comparison of the non-human and human mAbs in order to evaluate the impact of the humanization process into immunogenicity of the final product.

De-immunization is another approach developed to reduce the immunogenicity of chimeric or rat antibodies. It involves the identification of linear T-cell epitopes in the antibody of interest, using bioinformatics, and their subsequent replacement by site-directed mutagenesis to human or non-immunogenic sequence⁵⁹. Although de-immunized antibodies exhibited reduced immunogenicity in primates, compared with their chimeric counterparts, some loss of binding affinity was observed⁶⁰.

Antibodies that are chimeric, deimmunised, humanized, human-like, resurfaced or humanized monoclonal or human antibodies are particular useful for treatment protocols because such antibodies can reduce immunogenicity and thus avoid human anti-mouse antibody (HAMA) response. In certain conditions it is preferable that the antibody is virtually free of Fc effector function, such is for example the case for natural IgG4 or IgG2, or for genetically mutated IgG or IgM which does not augment antibody-dependent cellular cytotoxicity⁶¹ and complement mediated cytolysis^{62, 63}. In addition, in case of IgG4, it is preferred to use stabilized hinge forms of IgG4 to prevent the occurrence of half molecule formation⁶⁴ or other forms of IgG4 aimed at preventing half molecule formation. In other conditions, it is desired that antibodies have enhanced Fc effector function. Such enhanced Fc effector function can be achieved, for example, by Fc engineering. One example of technology used for Fc-engineering is the Xmab® Technology of Xencor (www.xencor.com).

Antibodies are glycoproteins containing between 3 and 12% carbohydrate. The carbohydrate units are transferred to acceptor sites on the antibody chains after the heavy and light chains have combined. The major carbohydrate units are attached to amino acid residues of the constant region of the antibody. The carbohydrate units may affect overall solubility and the rate of catabolism of the antibody. It is also known that carbohydrate is necessary for cellular secretion of some antibody chains. It has been demonstrated that glycosylation of the constant region plays a vital role in the effector functioning of an antibody; without this glycosylation in its correct configuration, the antibody may be able to bind to the antigen but may not be able to bind for example to macrophages, helper and suppressor cells or complement, to carry out its role of blocking or lysing the cell to which it is bound. The antibody of the invention may therefore be glycosylated in the cells in which it is produced to maintain antigen binding capability and effector functionality.

Antibodies produced according to a method of the invention may further differ in acetylation, pegylation, phosphorylation, and/or amidation, compared to the antibody produced by the hybridoma cell.

Antibodies of the invention, whether polyclonal or monoclonal, may have additional utility in that they may be employed as reagents in immunoassays, radioimmunoassays (RIA), enzyme-linked immunosorbent assays (ELISA) or protein arrays. In these applications, the antibodies can be labelled with an analytically-detectable reagent such as a radioisotope, a fluorescent molecule or an enzyme.

An example of an agonistic antibody is the antibody 1D9 (see Example 3). Therefore, in one embodiment, the agonist is the antibody 1D9 (also referred to herein as Ab1). The VL of antibody 1D9 is represented by SEQ ID NO: 131 and the VH is represented by SEQ ID NO: 129. Therefore, in one embodiment, the agonist is an antibody comprising or consisting of SEQ ID NO: 129 and/or SEQ ID NO: 131. The CDRs of antibody 1D9 are represented by SEQ ID NOs: 133-138. A hybridoma producing 1D9 has been deposited at the institute BCCM/LMBP on 28 Jul. 2009 with the following accession number: LMBP 6961CB.

In one embodiment the agonist is an antibody (or fragment or derivative thereof), wherein the sequence of the VH is at least 90%, at least 95%, at least 98%, or at least 99% identical to SEQ ID NO: 129 and/or the sequence of the VL is at least 90%, at least 95%, at least 98%, or at least 99% identical to SEQ ID NO: 131.

In some embodiments the agonist is a chimeric, deimmunised, humanized, human-like, resurfaced or humanized monoclonal or human antibody (or fragment or derivative thereof) wherein the sequence of the VH is at least 90%, at least 95%, at least 98%, or at least 99% identical to SEQ ID NO: 129 and/or the sequence of the VL is at least 90%, at least 95%, at least 98%, or at least 99% identical to SEQ ID NO: 131.

In one embodiment the agonist is an antibody (or fragment or derivative thereof), wherein the CDRs of said antibody (or fragment or derivative thereof) comprise one or more, preferably all of the CDR sequences of the 1D9 antibody or sequences that are at least 90%, at least 95%, at least 98%, or at least 99% identical to those sequences.

In some embodiments the agonist is a chimeric, deimmunised, humanized, human-like, resurfaced or humanized monoclonal or human antibody (or fragment or derivative thereof) wherein the CDRs of said antibody (or fragment or derivative thereof) comprise one or more, preferably all of the CDR sequences of the 1D9 antibody or sequences that are at least 90%, at least 95%, at least 98%, or at least 99% identical to those sequences.

Antibody 1D9 binds to the CRL region on Lgr5 (see section on “Binding of the agonist to the Lgr protein”). Other antibodies targeting the CRL region on Lgr4, Lgr5 or Lgr6 cells could also be expected to have agonistic activity. Therefore, in some embodiments of the invention, the agonist is an antibody that binds to the CRL region of an Lgr protein.

In some embodiments of the invention, the agonist of the invention is not antibody 1D9. This is particularly envisaged in some embodiments relating to the agonist per se. However, in other aspects of the invention, it is envisaged that 1D9 is encompassed by the invention, for example wherein the agonist is a multi-targeting compound and/or when the agonist or multi-targeting compound is present/used in for example pharmaceutical compositions, multi-targeting compounds, methods for treatment, methods for enhancing cell growth/proliferation and methods for identifying new agonists.

### Multi-Targeting Compounds

In some embodiments, the agonist of the invention is a multi-targeting compound as described herein. By “multi-targeting compound”, it is meant that the agonist of the invention additionally binds to at least one further target polypeptide or other target molecule. In some embodiments the other target molecule is a carbohydrate moiety. Therefore, in some embodiments the invention provides a multi-targeting compound, comprising at least one portion with Rspondin-mimicking activity, and at least one other portion that binds to a further target polypeptide or other target molecule. The phrases “agonist which is a multi-targeting compound” and “agonist of the invention which additionally binds to at least one further target polypeptide or other target molecule” are used interchangeably herein. For example, the agonist may bind to an Lgr protein and thus mimic the activity of Rspondin and additionally binding at least a second polypeptide or other target molecule, for example to confer tissue-specific targeting of the Rspondin-mimicking activity. In some aspects, the agonist may bind 2, 3, 4 or more polypeptides.

The further target polypeptide or other target molecule may for example be a Frizzled Receptor, for example Frz1, Frz2, Frz3, Frz4, Frz5, Frz6, Frz7, Frz8, Frz9 or Frz10. In one embodiment, the agonist may bind i) at least one of Lgr4, Lgr5 or Lgr6, and ii) at least one of Frz1-10. In particular, the agonist may bind i) at least one of Lgr4, Lgr5 or Lgr6, and ii) at least one of Frz5, Frz6 and Frz7. Such a compound might modulate activity of the Wnt pathway via both Lgr and Frz in the Wnt receptor complex, for example it may enhance Wnt signalling to a greater extent by mimicking the natural synergistic action of Rspondin and soluble Wnt molecules in vertebrate cells.

The further target polypeptide or other target molecule may be an LRP, for example LRP5 or LRP6. In one embodiment, the agonist may bind i) at least one of Lgr4, Lgr5 or Lgr6, and ii) at least one of LRP5-6. Such a compound might modulate activity of the Wnt pathway via both Lgr and LRP in the Wnt receptor complex. For example it may enhance Wnt signalling to a greater extent by mimicking the natural synergistic action of Rspondin and soluble Wnt molecules in vertebrate cells.

In an alternative embodiment, the further target polypeptide or other target molecule may be a second Lgr protein for example Lgr 4, 5 or 6, such that the agonist targets two or three Lgr proteins. For example, an agonist of the invention might bind Lgr4 and Lgr5, Lgr5 and Lgr 6, or Lgr4 and Lgr6, or Lgr4, Lgr5 and Lgr6. The combination of Lgr4 and Lgr5 antibodies in a mixture or as a bispecific may be particularly useful because the inventors have found that in many tissues Lgr5 expression is usually accompanied by Lgr4 expression (see Example 1). Therefore, an agonist that binds to both Lgr4 and Lgr5 may have a greater effect on enhancing Wnt signaling in these tissues.

Alternatively, the further target polypeptide or other target molecule might be a tissue-specific, or cell-specific marker for tissue-specific or cell-specific targeting. The agonist of the invention may thus bind an Lgr protein and a tissue-specific, or cell-specific marker. For example, for liver-specific targeting, an agonist of the invention may bind Lgr and L-cadherin. The agonist might also bind a further target polypeptide or other target molecule present in epithelial tissues, such as skin tissue, stomach lining, pancreatic lining, liver, kidney; connective tissues, such as inner layers of skin, tendons, ligaments, cartilage, bone, fat, hair, blood; muscle tissues; and nerve tissues, such as glial cells and neurons. Such markers include, but are not limited to membrane proteins that are specifically expressed on certain organs such as liver, pancreas or colon. These markers are well known in the art.

In other embodiments, the further target polypeptide or other target molecule may be a tissue-specific marker that is expressed on cells in the liver, pancreas, kidney, small intestine, colon etc. Such markers include, but are not limited to Epcam for epithelial cells, CA19 for pancreas, A33 for intestine, L-cadherin for the liver, dipeptidyl peptidase V for the pancreas and/or liver, L-SIGN for endothelial cells of the liver and lymph nodes (Gardner et al., 100(8) 4498-4503 PNAS 2002), CD-26 (also known as DPPIV) for the liver (in particular for ducts and hepatocytes), Integrin a6b1 for the liver (in particular for biliary ducts) (Couvelard et al., Hepatology 27(3), 839-847 (1998)). Therefore, a multi-targeting compound of the invention may target Lgr4 (which is expressed by all proliferating cells) and/or Lgr5 and/or Lgr6, and one or more of these tissue-specific markers.

In an alternative embodiment, the further target polypeptide or other target molecule may be a secreted molecule that is itself naturally (or otherwise) targeted to a tissue or cell of interest. For example, coagulation factor IX and complement component C4 are secreted molecules that are targeted to the liver.

In one embodiment, the multi-targeting compound has the effect of increasing the affinity of the Rspondin-mimicking activity for the target cell and/or tissue. An agonist used alone will diffuse throughout its environment (e.g. the body or cell culture) where it is used. This means that it will activate all cells with an Rspondin-responsive receptor (now known to include Lgr4, Lgr5 and Lgr6). Where Rspondin might be used for treatment purposes (such as those described later in the application) and is administered to an animal or patient, the dose would have to be kept low in order to minimise adverse effects on non-target organs/tissues/cells caused by non-specific binding of Rspondin to its receptors. Agonists that bind to a second or third (or more) target polypeptides that are specific for the target organ/tissue/cell, would require lower dosages to achieve similar, equal or better activation of the Wnt pathway in a target organ/tissue/cell whilst minimising the adverse effects on the non-target organs/tissues/cells. Therefore, in one embodiment, the further target polypeptide or other target molecule is a membrane molecule that is expressed on the target organ/tissue/cell of interest and optionally is not expressed on other non-target organs/tissues/cells and optionally not expressed on cells which do not have Rspondin-responsive receptors.

The present inventors have also surprisingly discovered that Lgr proteins are expressed basolaterally (see FIG. 27). Therefore, it is clearly advantageous to target a multi-targeting agonist to other basolaterally expressed targets. If the multi-targeting compound was targeted to the apical side of a tissue membrane, where Lgr proteins are not expressed, it would be unlikely to be able to perform its function as an agonist of the invention. Without this knowledge, the skilled person would not have known how to combine tissue-specific targeting with Lgr targeting and thus to generate the multi-targeting agonists as described herein. Therefore, in some embodiments the further target polypeptide or other target molecule is expressed basolaterally. In a further embodiment, the further target polypeptide is not expressed apically. Examples of basolaterally expressed targets include but are not limited to Epcam, A33 and L-cadherin.

The present inventors have surprisingly demonstrated that a Furin domain fragment (which mimics the activity of Rspondin binding to Lgr) linked to an anti-Epcam antibody can act as a substitute for Rspondin, even at 50× lower concentrations than Rspondin (see Example 4 and FIG. 24).

In some embodiments, the further target polypeptide or other target molecule is a marker that is specifically expressed in Lgr5+ cells. The isolation of Lgr5+ stem cells is described for the first time in WO2009/022907. Lgr5+ stem cells differ from all previously described stem cells in that they are not quiescent, they have high levels of telomerase, they produce their own niche and they can survive more than 1000 divisions in stem cell culture medium (see WO2010/090513). Therefore, these cells differ from every other population of stem cells previously described or isolated. It is envisaged that knowledge of Lgr5+ stem cell markers can be combined with the surprising finding that Rspondin activates Wnt signalling through the Lgr-LRP-Frz complex, to generate multi-targeting compounds that have high avidity for Lgr5+ stem cells in particular target tissues of interest. In some embodiments, a multi-targeting compound of the invention, may target an Lgr5+ stem cell marker and Lgr5 and/or Lgr4 and/or Lgr6. In a preferred embodiment, a multi-targeting compound that targets an Lgr5+ stem cell marker will also target Lgr5, optionally without also targeting Lgr4 and/or Lgr6. In other embodiments, the multi-targeting compound that targets an Lgr5+ stem cell marker, and optionally targets Lgr5, will also target Lgr4 and/or Lgr6.

In some embodiments the invention provides a multi-targeting compound, comprising at least one portion with Rspondin-mimicking activity, and at least one other portion that binds to a further target polypeptide or other target molecule.

In some embodiments of the invention, wherein the agonist is a multi-targeting compound, the agonist of the invention comprises an Rspondin protein. By this it is meant that in some embodiments, the at least one portion with Rspondin-mimicking activity comprises an Rspondin protein. The Rspondin protein according to the invention may be Rspondin 1, Rspondin 2, Rspondin 3 or Rspondin 4. In other embodiments of the invention, the multi-targeting compound comprises an Rspondin variant, derivative or fragment as described herein. In some embodiments, the agonist is an Rspondin derivative that is specific to only Lgr4, Lgr5 or Lgr6. For example the agonist in some embodiments the agonist is an Rspondin derivative specific to only Lgr5, for example by mutation of one or more amino acid residues involved in binding to the Lgr4, Lgr5 and Lgr6 proteins. In other embodiments of the invention, the multi-targeting compound comprises a small-molecule agonist of the invention or any other agonist of the invention described herein.

The multi-targeting compound retains useful functionality in terms of enhancing Wnt signalling by mimicking the activity of Rspondin binding to an Lgr protein.

By useful functionality, it is meant that the multi-targeting agonist of the invention enhances β-catenin signalling in a cell, group of cells or a tissue by an amount that is useful for the intended purpose of the agonist, for example, by at least 30%, at least 35%, at least 40%, at least 45%, at least 50%, at least 60%, at least 70%, at least 75%, at least 80%, at least 85%, at least 90%, at least 95%, at least 100%, at least 150%, at least 200%, at least 300%, at least 400% or at least 500% compared to the β-catenin signalling induced by a neutral substance or negative control as measured in an assay described herein, for example as measured in the TOPFlash assay.

In some embodiments, a multi-targeting agonist of the invention enhances β-catenin signalling in a cell, group of cells or a tissue by an amount that is useful for the intended purpose of the agonist, for example, by at least 10×, at least 20×, at least 50×, at least 100×, at least 200×, at least 300×, at least 400×, at least 500×, at least 1000×, at least 2000×, at least 5000×, or at least 10,000× compared to the β-catenin signalling induced by Rspondin as measured in an assay described herein, for example as measured in the TOPFlash assay.

In some embodiments, the Rspondin is Rspondin 4. Rspondin 4 is a non-essential protein in mice and humans. Therefore, it is expected to result in fewer negative side-effects when used in therapy.

In other embodiments, the at least one portion with Rspondin-mimicking activity is any agonist of the invention described herein.

In some embodiments the at least one other portion that binds to a further polypeptide or other target molecule is a polypeptide, a peptidomimetic, an antibody or a fragment thereof, an aptamer or a small molecule. For example, in some embodiments the antibody is an anti-Epcam antibody. In other embodiments the small molecule is coagulation factor IX or complement component C4. The anti-Epcam antibody targets epithelial cells (see Example 4). Coagulation factor IX and complement component C4 target the liver.

In some embodiments in which the agonist of the invention binds to a further target polypeptide or other target molecule, the agonist may be a conjugate or a fusion protein.

As used herein, the term “conjugate” refers to two or more molecules that have been covalently joined, optionally by a linking region. For example, in some embodiments, a conjugate is a first protein or non-protein moiety joined to a second protein or non-protein moiety by a linking region. For example, in some embodiments of the invention a multi-targeting agonist comprises or consists of two antibodies that have been covalently joined. A conjugate is not limited to a first and second moiety but in some embodiments may also have a third, fourth or more moieties joined by further linking regions. As described elsewhere in this application, examples of protein moieties include, but are not limited to: a polypeptide, a peptidomimetic or an antibody or fragment thereof. Examples of non-protein moieties include, but are not limited to: an aptamer or a small-molecule.

Numerous types of linker can be used, and the linker will be selected to be appropriate according to the molecule types in the conjugate and on the desired properties of the linker (length, flexibility, resistance to protease activity and other similar characteristics). Such linkers may comprise nucleotides, polypeptides, or a suitable synthetic material. For example, a linker may be a flexible peptide linker (for example, see SEQ ID NO: 142). In certain embodiments, the linker may be a cleavable linker, allowing the parts of the conjugate to be separated from each other. In other embodiments, a peptide linker might be a helical linker. Various examples and kits for linking proteins and other molecules are well known in the art.

As used herein, the term “fusion protein” refers to a protein that comprises two or more polypeptides or proteins that have been joined at the DNA level by recombination and are expressed together as a single polypeptide. A fusion protein may also comprise a peptide linking region also encoded by the DNA and expressed together with the fusion protein. A peptide linker that is part of a fusion protein, may be designed to have particular characteristics such as flexibility, hydrophilicity, protease-resistance, cleavability etc. All these properties can be designed within the DNA sequence and methods for designing linkers are well known in the art.

Where the agonist of the invention binds to a further target polypeptide or other target molecule (i.e. where it is a multi-targeting compound), it may be a bispecific antibody. A wide variety of multispecific recombinant antibody formats are known in the art, such as tetravalent bispecific antibodies by fusion of, e.g., an IgG antibody format and single chain domains^{65, 66, 67}. For example, TandAbs® (Affimed) are tetravalent bispecific antibody formats that have two binding sites for each antigen. They bind to target molecules on the surface of, for example, tumour cells and can activate immune effector cells like cytotoxic T-cells or natural killer (NK) cells. TandAbs® possess the same avidity and affinity for each target as an IgG. Similarly, BiTE's (Bispecific T-cell engager molecules) constitute a class of bispecific single-chain antibodies for the polyclonal activation and redirection of cytotoxic T cells against pathogenic target cells. Several other new formats wherein the antibody core structure (IgA, IgD, IgE, IgG or IgM) is no longer retained such as dia-, tria- or tetrabodies, minibodies, several single chain formats (scFv, Bis-scFv), which are capable of binding two or, more antigens, have also been developed^{68, 69, 70, 71}. All such formats use linkers either to fuse the antibody core (IgA, IgD, IgE, IgG or IgM) to a further binding protein (e.g. scFv) or to fuse e.g. two Fab fragments or scFvs⁶⁹.

In one embodiment, where the agonist of the invention binds to a further target polypeptide or other target molecule (i.e. where it is a multi-targeting compound), it comprises or consists of two or more antibodies. In a further embodiment, at least one antibody is an agonist according to the present invention and at least one antibody binds to a tissue-specific cell marker.

### Exemplary Multi-Targeting Agonists

In one embodiment, the multi-targeting compound is an agonist of the invention linked to an antibody that binds to a tissue-specific marker.

In one embodiment, the multi-targeting compound is Rspondin 1-4 linked to an antibody that binds a tissue-specific marker.

In one embodiment, the multi-targeting compound is an Rspondin fragment, for example a Furin domain fragment, linked to an antibody that binds a tissue-specific marker.

In one embodiment the Rspondin fragment is represented by the sequence of amino acids recited in SEQ ID NO: 139 or SEQ ID NO: 140, SEQ ID NO: 141 or SEQ ID NO: 143.

In one embodiment, the multi-targeting compound comprises or consists of 1D9 linked to an antibody that binds a tissue-specific marker.

In one embodiment, the multi-targeting compound is a bispecific antibody which has a first portion with agonistic activity according to the invention i.e. a first portion that mimics the activity of Rspondin binding to an Lgr protein, and a second portion that binds to a tissue-specific marker.

In a further embodiment the tissue-specific marker is selected from the list comprising: Epcam, A33, L-cadherin, CA19, dipeptidyl peptidase V, L-SIGN for endothelial cells of the liver and lymph nodes, CD-26 (also known as DPPIV), and Integrin a6b1.

### Antagonists

Alternatively, the invention provides a bispecific compound, optionally a bi-specific antibody, which binds to both Lgr4 and Lgr5 or Lgr4 and Lgr6 or Lgr5 and Lgr6 and inhibits Wnt/β-catenin signalling. Similarly, the invention also provides mixtures of compounds that inhibit Rspondin binding to Lgr4 and Lgr5 or Lgr4 and Lgr6 or Lgr5 and Lgr6, optionally mixtures comprising anti-Lgr4 and anti-Lgr5 or anti-Lgr4 and anti-Lgr6 or anti-Lgr5 and anti-Lgr6 antibodies that inhibit Wnt/β-catenin signalling.

Antibodies that are antagonists of Lgr4 or Lgr5 and/or Lgr6 are known in the art (for example, see WO 2010016766), and can be easily generated by methods well-known in the art. For example, the inventors have demonstrated that the following anti-Lgr5 antibodies are antagonists of the Wnt pathway: 6C10, 2B6, 3B9, 8F2 and 9G5 (see FIG. 28A). The inventors have demonstrated that the following anti-Lgr4 antibodies are antagonists of the Wnt pathway: 5C4, 4D4, 7F8, 4B4, 3C6, 3A11, 9B3, 2H10, 6C7, 8H9, 9B8 (see FIG. 28B).

The inventors have discovered that antagonistic antibodies often bind to the N-terminal domain and/or one or more LRR of the Lgr protein, whereas examples of agonistic antibodies have been seen to target the CRL region (see FIG. 4C in combination with FIG. 28A and table 4). This information may be useful for the generation and/or design of new antagonists (and agonists) of Wnt/β-catenin signalling, including antagonistic antibodies (see the section “Methods for identifying further agonists”). In some embodiments, therefore, the anti-Lgr antibody that inhibits Wnt/β-catenin signalling, binds to the N-terminal domain and/or one or more LRR.

Antagonistic antibodies can be linked together by methods well-known in the art, and as described herein, to form bispecific or multi-targeting antagonists. Alternatively, they can be administered in combination. Where the antibodies are administered in combination they may be administered simultaneously, sequentially, or separately in any order.

Compounds which inhibit Wnt/β-catenin signalling are generally considered to show promise as therapeutics for cancer treatment. In WO 2009/005809 an Lgr5 antibody is described for possible treatment of cancer. However, in tissues where Lgr5 is always accompanied by Lgr4 expression, inhibition of Lgr5 alone would be insufficient for cancer treatment because, as is shown for the first time in the present application (see example 1), the inhibited Lgr5 is redundant and so Rspondin-mediated signalling could still occur through Lgr4. Therefore, a compound, or a mixture of compounds, that targets both Lgr4 and Lgr5 in these tissues would be clearly advantageous over a compound only targeting Lgr5, for effectively blocking Wnt/β-catenin signalling and reducing cellular proliferation for the treatment of cancer. The same reasoning also applies for Lgr6, which is also often expressed in combination with Lgr4.

In some embodiments, one or more of the compounds that inhibit Rspondin binding to Lrg4 or Lgr5 or Lgr6 (i.e. which bind to and “inhibit” the activation of Lgr4, Lgr5 and/or Lgr6) are selected from the group consisting of: a small-molecule, a polypeptide, an antibody or fragment thereof, a biological antagonist, an aptamer and an antisense oligonucleotide. As used herein, a biological antagonist of Lgr4 or Lgr5 or Lgr6 refers to a compound that inhibits Lgr4 or Lgr5 or Lgr6 that occurs naturally in biological cells, for example, a biological antagonist may be a protein or a siRNA.

Accordingly, the invention provides a method of inhibiting β-catenin signalling comprising administering a bispecific antibody or a mixture of compounds that inhibit Lgr4 and Lgr5 or Lgr4 and Lgr6 or Lgr5 and Lgr6. The method may be conducted in vitro, in vivo or ex vivo. Preferably, the method is conducted in vivo and is administered to a patient to treat cancer.

Also provided is a method of inhibiting β-catenin signalling comprising administering a compound that inhibits Rspondin binding to Lgr4 in combination with a compound that inhibits Rspondin binding to Lgr5, or a compound that inhibits Rspondin binding to Lgr4 in combination with a compound that inhibits Rspondin binding to Lgr6, or a compound that inhibits Rspondin binding to Lgr5 in combination with a compound that inhibits Rspondin binding to Lgr6, wherein said compounds are administered simultaneously, sequentially, or separately in any order.

Thus the invention provides a method for treating disorders associated with abnormal tissue growth such as cancer, comprising administering one, two or all three of a compound that inhibits Lgr5, a compound that inhibits Lgr4 and a compound that inhibits Lgr6 to a patient, wherein the compounds are administered simultaneously, sequentially or separately in any order.

The invention also provides a compound that inhibits Lgr4 for use in treating a patient who has previously been treated with a compound that inhibits Lgr5. Conversely, the invention also provides a compound that inhibits Lgr5 for use in treating a patient who has previously been treated with a compound that inhibits Lgr4.

The invention also provides a compound that inhibits Lgr4 for use in treating a patient who has previously been treated with a compound that inhibits Lgr6. Conversely, the invention also provides a compound that inhibits Lgr6 for use in treating a patient who has previously been treated with a compound that inhibits Lgr4.

The invention also provides a compound that inhibits Lgr6 for use in treating a patient who has previously been treated with a compound that inhibits Lgr5. Conversely, the invention also provides a compound that inhibits Lgr5 for use in treating a patient who has previously been treated with a compound that inhibits Lgr6.

In some embodiments, the invention also provides a kit comprising a compound that inhibits Lgr5 and a compound that inhibits Lgr4. In some embodiments, the invention also provides a kit comprising a compound that inhibits Lgr5 and a compound that inhibits Lgr6. In some embodiments, the invention also provides a kit comprising a compound that inhibits Lgr4 and a compound that inhibits Lgr6. In some embodiments, the invention also provides a kit comprising a compound that inhibits Lgr4 and a compound that inhibits Lgr6 and a compound that inhibits Lgr5. The compound that inhibits Lgr5 and/or the compound that inhibits Lgr4 and/or the compound that inhibits Lgr6 may be for simultaneous, sequential or separate administration. In a further embodiment, the kit of the invention is for use in treating a patient. In a further embodiment, the kit of the invention is for use in accordance with any of the methods of the invention, wherein the methods comprise inhibiting β-catenin signalling.

Cancers that would benefit from combined Lgr4/5 modulation provided by mixtures or bispecifics of the present invention, would be cancers that lack mutationally activated Wnt signalling, i.e. cancers that have normal Wnt signaling. Examples of such cancers include: breast cancer, ovarian cancer, lung cancer, prostate cancer, or skeletal cancer (e.g. osteosarcoma). It is believed that in cancers wherein the intracellular Wnt signalling is disturbed (mutationally activated, for instance APC mutations in colon cancer) blocking Lgr4 or Lgr5 would have no effect.

Although bispecific compounds have been described in detail, it is to be understood that multi-targeting antagonists would also be possible. For example, in one embodiment the invention provides a multi-targeting antagonist that inhibits activation of Lgr4, Lgr5 and Lgr6. In one embodiment, the multi-targeting antagonist may comprise of an anti-Lgr4 antibody, an anti-Lgr5 antibody and an anti-Lgr6 antibody. In some embodiments, the multi-targeting antagonists inhibit activation by preventing Rspondin, and optionally an Rspondin-mimicking agonist, from binding to Lgr4, Lgr5 and/or Lgr6, or alternatively from activating Lgr4, Lgr5 and/or Lgr6.

In a further embodiment the invention provides antagonists that block activation of the Wnt pathway by binding to the Rspondin binding site or the antibody 1D9 binding site. For example, the invention provides antagonists that block the activity of an agonist binding to the CRL region or to the LRRs of an Lgr protein.

### Compositions

As discussed in more detail below, the agonists of the invention have widespread medical applications. The invention therefore further provides a pharmaceutical composition comprising one or more agonist according to the invention and a pharmaceutically acceptable carrier or excipient. Pharmaceutically acceptable carriers and excipients are known in the art and described, for instance, in “Remington; The Science and Practice of Pharmacy”⁷².

The one or more agonists according to the invention may be any combination of agonists that mimic the effect of Rspondin binding to Lgr4, Lgr5 or Lgr6 (also referred to herein as Lgr4, Lgr5 and Lgr6 agonists), including for example small molecules, antibodies, multi-targeting compounds etc as described herein. A composition of the invention, for example, comprising an Lgr4 agonist of the invention and an Lgr5 agonist of the invention might be expected to be more effective at enhancing Wnt signalling in certain tissues, compared to a composition only containing an Lgr5 agonist. Other combinations of agonists may have other beneficial effects according to the particular tissue-specific expression patterns of Lgr4, Lgr5 and Lgr6, outlined elsewhere in the application.

In one embodiment, the compositions comprising one or more agonists of the invention may comprise one or more antibody for Lgr4, Lgr5 or Lgr6 according to the invention, or any combination of the above. For example, the composition may comprise an agonistic anti-Lgr4 antibody and an agonistic anti-Lgr5 antibody. Alternatively, the composition may comprise an agonistic anti-Lgr4 antibody and an agonistic anti-Lgr6 antibody. Alternatively, the composition may comprise an agonistic anti-Lgr5 antibody and an agonistic anti-Lgr6 antibody. Alternatively, the composition may comprise an agonistic anti-Lgr4 antibody, an agonistic anti-Lgr5 antibody and an agonistic anti-Lgr5 antibody.

In one embodiment, the compositions comprising one or more agonists of the invention, also comprise Wnt and/or a suitable Wnt substitute that binds and activates the Wnt pathway via Frizzled and LRP.

The compositions comprising one or more agonists of the invention may be used to treat a range of disorders associated with tissue loss or damage due to aging or pathological conditions. The pharmaceutical compositions of the invention may therefore include other drugs known to be useful in the treatment of these conditions. The pharmaceutical composition further comprises an effective amount of at least one compound or protein selected from at least one of: an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, or a nutritional drug.

The invention also provides a pharmaceutical composition comprising two or more antagonists according to the invention and a pharmaceutically acceptable carrier or excipient. The two or more antagonists may be any combination of compounds that inhibit binding of Rspondin to Lgr4, Lgr5 or Lgr6. In a preferred embodiment, the two or more antagonists inhibit binding of Rspondin to Lgr4 and Lgr5.

The compositions comprising one or more antagonists of the invention may be used to treat a range of disorders in which it is desirable to inhibit/antagonise Wnt signalling, for example disorders associated with abnormal tissue growth, such as cancer. The pharmaceutical compositions of the invention may therefore include other drugs known to be useful in the treatment of these conditions. The pharmaceutical composition further comprises an effective amount of at least one compound or protein selected from at least one of: an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or a cancer drug, such as Tamoxifen.

The compositions of the invention may be formulated so that it is suitable for administration by any administration routes known in the art, for example intravenous, subcutaneous, intramuscular administration, mucosal, intradermal, intracutaneous, oral, and ocular. A pharmaceutical composition may be thus be in any form suitable for such administration, e.g. a tablet, infusion fluid, capsule, syrup, etc.

### Method of Treatment

The present invention provides an agonist or a composition according to the invention for use in therapy. In some embodiments, the invention provides an agonist or a composition comprising one or more agonists according to the invention for use in treating diseased or damaged tissue, for use in tissue regeneration and for use in cell growth and proliferation, and/or for use in tissue engineering. In particular, the present invention provides an agonist or a composition comprising one or more agonists according to the invention for use in treating tissue loss or damage due to aging or pathological conditions.

In some embodiments of the invention, the agonist of composition for use in therapy comprises or consists of antibody 1D9 and/or a multi-targeting compound comprising an anti-Epcam antibody linked to a Rspondin Furin domain fragment.

The invention also provides a method for treating tissue loss or damage due to aging or pathological conditions by administering an agonist or composition comprising one or more agonists of the invention to a patient. The patient may be any animal (e.g., a mammal), including, but not limited to, humans, non-human primates, rodents, and the like. Typically, the patient is human. The methods of treatment and medical uses of the agonists of the invention or compounds or compositions comprising agonists of the invention promote tissue regeneration. The term “tissue” refers to part of an organism consisting of a cell or an aggregate of cells, optionally having a similar structure, function and/or origin. Examples of tissues include but are not limited to: epithelial tissues, such as skin tissue, stomach lining, pancreatic lining, liver; connective tissues, such as inner layers of skin, tendons, ligaments, cartilage, bone, fat, hair, blood; muscle tissues; and nerve tissues, such as glial cells and neurons. The loss or damage can be anything which causes the cell number to diminish. For example, an accident, an autoimmune disorder, a therapeutic side-effect or a disease state could constitute trauma. Specific examples of conditions which may cause cell number to diminish include, but are not limited to: radiation/chemotherapy, mucositis, IBD, short bowel syndrome, hereditary bowel disorders, celiac disease, metabolic diseases, hereditary syndromes, (viral) infections (hepB/C), toxic states, alcoholic liver, fatty liver, cirrhosis, infections, pernicious anemia, ulceration, diabetes, destruction of islet cells, loss of bone mass (osteoporosis), loss of functional skin, loss of hair, loss of functional lung tissue, loss of kidney tissue (for instance acute tubulus necrosis), loss of sensory cells in the inner ear. Tissue regeneration increases the cell number within the tissue and preferably enables connections between cells of the tissue to be re-established, and more preferably the functionality of the tissue to be regained.

Other conditions that may be treated with the agonists or compositions comprising one or more agonists of the invention include but are not limited to: joint disorders, osteoporosis and related bone diseases, baldness, graft-versus-host disease.

Agonists or compositions comprising one or more agonists of the invention, in particular agonists that bind and activate Lgr6, may also be used for wound healing and generation of smooth muscle tissues in many organs (e.g. airways, large arteries, uterus). In some embodiments, the invention provides methods of treatment and medical uses, as described previously, wherein two or more agonist of the invention or compounds or compositions comprising agonists of the invention, are administered to an animal or patient simultaneously, sequentially, or separately.

In some embodiments, the invention provides methods of treatment and medical uses, as described previously, wherein one or more agonist of the invention or compounds or compositions comprising agonists of the invention, is administered to an animal or patient in combination with one or more further compound or drug, and wherein said agonist of the invention or compounds or compositions comprising agonists of the invention and said further compound or drug are administered simultaneously, sequentially, or separately.

Examples of further compounds or drugs include but are not limited to: an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, or a nutritional drug.

In one embodiment, the one or more further compound or drug comprises or consists of Wnt and/or a suitable Wnt substitute that binds and activates the Wnt pathway via Frizzled and LRP.

### Methods for Enhancing Cell Growth/Proliferation

The agonists of the invention also have widespread applications in non-therapeutic methods, for example in vitro research methods.

The invention thus provides a method for tissue regeneration of damaged tissue, such as the tissues discussed in the section of medical uses above, comprising administering an agonist of the invention. The agonist may be administered directly to the cells in vivo, administered to the patient orally, intravenously, or by other methods known in the art, or administered to ex vivo cells. In some embodiments where the agonist of the invention is administered to ex vivo cells, these cells may be transplanted into a patient before, after or during administration of the agonist of the invention.

The invention also provides a method for enhancing the proliferation of cells comprising supplying the cells with an agonist of the invention.

These methods may be carried out in vivo, ex vivo or in vitro.

Wnt agonists are key components of stem cell culture media. For example, the stem cell culture media as described in WO2010/090513, WO2012/014076, Sato et al., 2011 (GASTROENTEROLOGY 2011; 141:1762-1772) and Sato et al., 2009 (Nature 459, 262-5) comprise Rspondin. The agonists of the invention that mimic Rspondin are thus suitable alternatives to Rspondin for use in these stem cell culture media. No alternatives to Rspondin in this context are already known.

The inventors have shown that antibody 1D9 can act as a substitute for Rspondin for culturing cells (see Example 3 and FIG. 23). The inventors have also shown that a multi-targeting compound comprising an anti-Epcam antibody linked to a Rspondin Furin domain fragment can act as a substitute for Rspondin for culturing cells. Furthermore, they have shown that this multi-targeting compound has a similar effect to Rspondin on cell growth even at 50× lower concentrations (see Example 4 and FIG. 24).

Accordingly, in one embodiment, the invention provides a method for enhancing the proliferation of stem cells comprising supplying stem cells with an agonist of the invention. In one embodiment, the invention provides a cell culture medium comprising one or more agonist of the invention. In some embodiments, the cell culture medium may be any cell culture medium already known in the art that normally comprises Rspondin, but wherein the Rspondin is replaced (wholly or partially) or supplemented by an agonist of the invention which mimics the activity of an Rspondin protein binding to an Lgr protein. For example, the culture medium may be as described in as described in WO2010/090513, WO2012/014076, Sato et al., 2011 (GASTROENTEROLOGY 2011; 141:1762-1772) and Sato et al., 2009 (Nature 459, 262-5), which are hereby incorporated by reference in their entirety.

For example, in one embodiment the invention provides a method for enhancing the proliferation of stem cells comprising supplying stem cells with antibody 1D9. In one embodiment, the invention provides a cell culture medium comprising antibody 1D9.

In another embodiment the invention provides a method for enhancing the proliferation of stem cells comprising supplying stem cells with a multi-targeting compound comprising an anti-Epcam antibody linked to a Rspondin Furin domain fragment. In one embodiment, the invention provides a cell culture medium comprising a multi-targeting compound comprising an anti-Epcam antibody linked to a Rspondin Furin domain fragment.

In a further embodiment the cell culture medium comprising one or more agonists of the invention, further comprises a receptor tyrosine kinase ligand (for example a growth factor, such as EGF) and a BMP inhibitor (for example Noggin). Such cell culture media are appropriate for growing organoids comprising Lgr5+ cells.

In a further embodiment, the cell culture medium comprising one or more agonists of the invention also comprises Wnt and/or a suitable Wnt substitute that binds and activates the Wnt pathway via Frizzled and/or LRP.

Stem cell culture media often comprise additional growth factors. This method may thus additionally comprise supplying the stem cells with a growth factor.

Growth factors commonly used in cell culture medium include epidermal growth factor (EGF, (Peprotech), Transforming Growth Factor-alpha (TGF-alpha, Peprotech), basic Fibroblast Growth Factor (bFGF, Peprotech), brain-derived neurotrophic factor (BDNF, R&D Systems), Human Growth Factor (HGF) and Keratinocyte Growth Factor (KGF, Peprotech, also known as FGF7). EGF is a potent mitogenic factor for a variety of cultured ectodermal and mesodermal cells and has a profound effect on the differentiation of specific cells in vivo and in vitro and of some fibroblasts in cell culture. The EGF precursor exists as a membrane-bound molecule which is proteolytically cleaved to generate the 53-amino acid peptide hormone that stimulates cells.

EGF or other mitogenic growth factors may thus be supplied to the stem cells. During culturing of stem cells, the mitogenic growth factor may be added to the culture medium every second day, while the culture medium is refreshed preferably every fourth day. In general, a mitogenic factor is selected from the groups consisting of: i) EGF, TGF-α and KGF, ii) EGF, TGF-α and FGF7; iii) EGF, TGF-α and FGF; iv) EGF and KGF; v) EGF and FGF7; vi) EGF and a FGF; vii) TGF-α and KGF; viii) TGF-α and FGF7; ix) or from TGF-α and a FGF.

These methods of enhancing proliferation of stem cells can be used to grow new organoids and tissues from stem cells, as for example described in WO2010/090513 WO2012/014076, Sato et al., 2011 (GASTROENTEROLOGY 2011; 141:1762-1772) and Sato et al., 2009 (Nature 459, 262-5).

In some embodiments the invention provides organoids obtained using the methods and/or media of the invention.

### Methods for Identifying Further Agonists

The identification of the mechanism by which Rspondin induces activation of the Wnt signalling pathway will also enable the identification of further agonists that mimic Rspondin activity. The identification of 1D9 as an agonist of Lgr5, and knowledge of the region where 1D9 binds on Lgr5 will also help identify and engineer new agonists of Lgr5, Lgr6 and Lgr4.

Based on the molecular structures of the variable regions of an anti-Lgr4, Lgr5 and/or Lgr6 antibody of the invention, molecular modelling and rational molecular design can be used to generate and to screen molecules which mimic the molecular structures of the binding region of the antibodies and thus mimic the activity of Rspondin binding to the Lgr proteins. These small molecules may be peptides, aptamers, peptidomimetics, oligonucleotides, antibodies or other organic compounds. Alternatively, one could use large-scale screening procedures commonly used in the field to isolate suitable molecules from libraries of compounds.

Common mutational techniques may be used to modify antibody 1D9 to generate new agonists with improved properties (e.g. in terms of increased beiding affinity to an Lgr protein, increased activity in terms of activating the Wnt pathway, reduced toxicity, greater solubility etc.). In addition, now that it is known that antibody 1D9 is an agonist and binds to the hinge (CRL) region of Lgr5 (see sections “Binding of the agonist to Lgr” and “Exemplary agonists” above), it is also possible to isolate the hinge region of Lgr4, Lgr5 or Lgr6 (or similar fragments comprising or consisting of the 1D9 binding sites) and to generate antibodies that bind to this specific region by methods well known in the art. The isolate hinge region of Lgr4, Lgr5 or Lgr6 (or similar fragments comprising or consisting of the 1D9 binding site) could also be used to screen for small molecules that bind this specific region by methods well known in the art. It would be expected that such antibodies or small molecules that bind this specific region would be more likely to behave as agonists and may also be more likely to be specific for Lgr5 (or Lgr4 or Lgr6).

Furthermore, now that the interaction between Lgr and Rspondin is known, and now that the agonistic activity of antibody 1D9 on Lgr5 is known, crystal structures, NMR or other structural information about the interacting amino acids that are involved in the Lgr-Rpsondin interaction and/or the Lgr-antibody (e.g. Lrg5-1D9) interaction, and/or site specific mutagenesis could be used to identify key interacting amino acids.

For example, an Rspondin derivative or fragment may be engineered to have increased specificity for Lgr5 compared to Lgr4 or Lgr6. Alternatively, an Rspondin derivative or fragment may bind and activate only Lgr5 and not Lgr4 or Lgr6. In an alternative embodiment, the Rspondin derivative or fragment is specific for, or has greater specificity for Lgr4 (relative to Lgr5 and/or Lgr6) or Lgr6 (relative to Lgr4 and/or Lgr5). This would be advantageous for use in therapy, for example, because wild-type Rspondin binds to Lgr4, Lgr5 and Lgr6 and stimulates growth, via the Wnt pathway, in all proliferating cells. An Rspondin derivative or fragment that has increased specificity for Lgr5, would preferentially stimulate growth of Lgr5-positive stem cells, not in all dividing cells. Therefore, in situations where wild-type Rspondin causes too much regrowth, an Rpsondin derivative or fragment with specificity for Lgr5 would be more appropriate for regenerative treatments.

The invention thus provides a method for identifying an agonist of the Wnt pathway, said method comprising:
a) Isolating a peptide fragment of an Lgr protein comprising or consisting of the region of Lgr that binds 1D9 and/or Rspondin;
b) Raising an antibody against said peptide fragment;
c) Testing said antibody for agonistic activity.

In an alternative embodiment, invention provides a method for identifying an antagonist of the Wnt pathway, said method comprising:
a) Isolating a peptide fragment of an Lgr protein comprising or consisting of the region of Lgr that binds 1D9 and/or Rspondin;
b) Raising an antibody against said peptide fragment;
c) Testing said antibody for antagonistic activity.

The invention also thus provides a method for identifying an agonist of the Wnt pathway, said method comprising:
a) contacting a complex comprising at least one Lgr protein, at least one Frizzled receptor and at least one LRP protein with a candidate compound in the presence of a Wnt protein; and
b) determining the level of Wnt/beta catenin signalling
wherein an increase in the level of Wnt/beta catenin indicates that the candidate compound is an agonist of the Wnt pathway.

The method may be a cell-based assay conducted in vitro.

The Lgr protein is at least one of Lgr4, Lgr5 or Lgr6. The Frz is at least one of Frizzled 1-10, typically at least one of Frz5, Frz6 and Frz7. The LRP co-receptor in the complex may be any LRP protein family member. The complex much thus comprise at least one of LRP5 or LRP6. The Wnt protein may be any Wnt family member.

The complex may comprise, for example, Wnt and Frz5-LRP6, Frz6-LRP6, Frz7-LRP6 and other combinations of the proteins described above. In particular, the Lgr protein may be Lgr4, Lgr5 and/or Lgr6 in a complex comprising Frz5, Frz6, and/or Frz7, and LRP5 and/or LRP6, as well as Wnt.

In some embodiments the Lgr protein in the method for identifying an agonist of the Wnt pathway may be a fragment of an Lgr protein. For example, in some embodiments, the fragment of Lgr comprises or consists of the hinge region (or CRL region) of Lgr4, Lgr5 or Lgr6.

An agonist in this context is a compound that mimics the activity of Rspondin, for example by binding an Lgr protein selected from Lgr4, Lgr5 or Lgr6, on a cell and initiates a reaction or activity that is similar to or the same as that initiated by the receptor's natural ligand Rspondin i.e. the agonist enhances Wnt signalling in the cell.

An increase in the level of Wnt/β-catenin signalling according to this screening method may be detected by identification of an increase in any of the following responses indicative of Wnt signalling: β-catenin stability, transcription of TCF-induced genes, LRP phosphorylation, axin translocation from cytoplasm to cell membrane and binding to LRP. Methods for determining the level of Wnt/beta-catenin signalling of candidate compounds are discussed in detail above and any of these assays may be used in the method of this aspect of the invention to assess whether a candidate compound is a Wnt agonist.

The candidate compounds can be obtained using any of the numerous approaches in combinatorial library methods known in the art, including biological libraries, peptoid libraries, spatially addressable parallel solid phase or solution phase libraries, synthetic library methods requiring deconvolution, the “one-bead one-compound” library method, and synthetic library methods using affinity chromatography selection. The biological library and peptoid library approaches are preferred for use with peptides, while the other four approaches are applicable to peptides, non-peptide oligomers or small molecules. Examples of methods for the synthesis of molecular libraries can be found in the art^{73, 74, 75, 76, 77, 78, 79}.

Libraries of compounds can be presented in solution⁸⁰, or on beads⁸¹, chips⁸², bacteria or spores⁸³, plasmids⁸⁴ or on phage^{85, 86, 87, 88}.

The candidate compounds may also be antibodies, such as the antibodies described above.

The candidate compounds may be selected by conducting a preliminary step of screening for compounds that bind to the Lgr protein and in particular that bind to the agonistic epitopes of the Lgr proteins discussed above.

Preferably, the method of screening for compounds that bind Lgr includes: (a) mixing Lgr and one or more candidate compounds; (b) incubating the mixture to allow Lgr and the candidate compound(s) to interact; and (c) assessing whether the candidate compound binds to the Lgr protein.

This method may be conducted when the Frz receptors and LRP co-receptors described above are also present, and in particular when the Lgr protein is bound in a complex with Frz receptors and LRP co-receptors. This complex would mimic the “Wnt receptor complex” that comprises Lgr proteins, Frz receptors and LRP co-receptors and that has now been identified by the inventors

Various assays for assessing binding of candidate compounds to Lgr are well known in the art, such as BIA, SPR, ELISA, FRET, Kinexa, FACS, and mass spectrometry.

Biomolecular interaction analysis (also known as BIA e.g., BIAcore 3000, ProteOn XPR36) utilises surface plasmon resonance to detect biospecific interactions in real time, without the need to label any of the interactants⁸⁹. Changes in the mass at the binding surface indicate a binding event and are measurable in terms of alterations of the refractive index of light near the binding surface. BIA may be used to characterise any kind of biomolecular interaction, including interactions between DNA-DNA, DNA-protein, lipid-protein and hybrid systems of biomolecules and non-biological surfaces can be investigated. Biomolecular Interaction Analysis can be used, for example, to identify the binding of two or more interactants to each other, to determine the affinity of the interactions, to measure the actual association and dissociation rates, and to determine whether two compounds bind competitively or cross-block in a competition binding assay.

The terms “cross-block”, “cross-blocked” and “cross-blocking” are used interchangeably herein to mean the ability of an antibody or other binding agent to interfere with the binding of other antibodies or binding agents to a certain epitope in a standard competitive binding assay. The ability or extent to which an antibody or other binding agent is able to interfere with the binding of another antibody or binding molecule to an epitope, and therefore whether it can be said to cross-block according to the invention, can be determined using standard competition binding assays.

The sandwich ELISA assay is an enzyme-linked immunosorbent assay which involves the immobilisation of a candidate compound on a solid support, such as a polystyrene microtitre plate. The antigen (e.g. an Lgr protein) is added and allowed to interact with the candidate compound. After washing with mild detergent solution to remove any protein that is not specifically bound, a detection antibody which is specific for the antigen is added. The detection antibody can be covalently linked to an enzyme, or can itself be detected by a secondary antibody that is linked to an enzyme through bioconjugation. After another wash step, the plate is developed by adding an enzymatic substrate to produce a visible signal, which indicates the quantity of antigen in the sample. A number of variations of the ELISA assay exist and are well known to those skilled in the art. For example, the competition ELISA assay relies on the ability of a labeled analogue to compete with the test sample analyte for a limited number of binding sites on a common binding partner. The amount of test sample analyte is inversely proportional to the amount of bound tracer as measured by the amount of marker substance.

The KinExA method is in particular useful for determining binding kinetics. The KinExA method measures the concentration of receptor (e.g. Lgr) molecule in a mixture of receptor, ligand (e.g. candidate compound), and ligand-receptor complex. The concentration of uncomplexed receptor is measured by exposing the solution phase mixture to solid phase immobilized ligand for a very brief period of time. The “contact time” between the solution phase mixture and the solid phase immobilized ligand is kept short enough that dissociation of ligand-receptor complex is insignificant. When the possibility of significant dissociation of ligand-receptor complex is kinetically excluded, only uncomplexed (“free”) receptor can bind to the solid phase. The amount of free receptor that binds to the solid phase (measured by fluorescence emission from a secondary label) is directly proportional to the concentration of free receptor in the solution phase sample.

The interaction between two molecules can also be detected using Förster resonance energy transfer (FRET)^{90, 91}. FRET is the phenomenon wherein a donor fluorophore, initially in its electronic excited state, may transfer energy to an acceptor fluorophore in close proximity through nonradiative dipole-dipole coupling. The first compound of interest (e.g. the candidate compound) is labeled with a donor and the second compound of interest (e.g. the Lgr protein) is labeled with an acceptor. When they are dissociated, the donor emission is detected upon the donor excitation. However, when the donor and acceptor are mixed and come into close proximity (1-10 nm) due to the interaction of the two molecules, the acceptor emission is predominantly observed because of the intermolecular FRET from the donor to the acceptor. An FRET binding event can be conveniently measured through standard fluorometric detection means well known in the art (e.g., using a fluorometer).

Affinity purification mass spectrometry is also a well-known approach for the characterisation of protein complexes (reviewed by Bauer, A. & Kuster, B.⁹²)⁹³. Protein complexes may first be isolated by a multitude of different techniques ranging from size exclusion or ion exchange chromatography to different varieties of affinity chromatography. For example, the protein complexes may be captured on affinity columns by antibodies specific for the protein of interest, or by more generic antibodies that capture fusion proteins containing epitope tags, such as Myc, HA, Flag, KT3. Tandem affinity purification employs a similar technique, except that the protein of interest is given two tags, and two affinity steps are used which reduces the amount of unspecific protein binding. Once the protein complex has been isolated, the component parts are then identified. Characterisation of the proteins in the complex may be carried out by mass spectrometry. A preferred approach is tandem affinity purification followed by mass spectrometry (see example 1). A number of mass spectrometry approaches may be employed and are well known and reviewed in the art^{94, 95}.

Once a candidate compound has been identified in vitro as a compound that is an agonist of the Wnt pathway, it may be desirable to perform further experiments to confirm the in vivo function of the compound.

Any of the above methods may therefore comprise the further steps of administering to a non-human animal a candidate compound and assessing its effect on enhancing cellular proliferation, for example, in terms of tissue regeneration.

The invention also provides a method of assessing the in vivo effect on cellular proliferation of a compound obtained or obtainable by any of the methods described above, comprising administering the compound to a human or animal and assessing the effect on cellular proliferation, for example, in terms of tissue regeneration.

### General

“GI” numbering is used above. A GI number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus the sequence associated with a given GI number is never changed.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Various aspects and embodiments of the invention are described below in more detail by way of example. It will be appreciated that modification of detail may be made without departing from the scope of the invention.

### DESCRIPTION OF THE DRAWINGS

FIG. 1: Conditional deletion of Lgr4 and Lgr5 in mouse small intestine.

FIG. 1A: Top: Expression of Lgr5-LacZ is specific to small stem cells located between Paneth cells at crypt bottoms. Bottom: Expression of Lgr4-lacZ⁹⁶ occurs throughout intestinal crypts (note lower magnification of right panel).

FIG. 1B: Design of Lgr5^{fl} allele. Black triangles indicate the inserted loxp sites. PMCNeo was removed by cre-mediated deletion in the germline.

FIG. 1C: Adult mice homozygous for Lgr4^{fl} and/or Lgr5^{fl}, and carrying the Ah-Cre transgene, were analyzed five days post-β-naphtoflavone-induced deletion⁹⁷. Left/middle panels: Proliferative cells are visualized by Ki67 staining. Right panels: Stem cells are visualized by OlfM4 in situ hybridization. Deletion of Lgr5 has no obvious effect. Deletion of Lgr4 has significant deleterious effects on crypt stem cells and proliferative progenitors as evidenced by loss of Ki67 crypts and Olfm4 signals. Upon Lgr4/5 double deletion, >80% of crypts disappear as visualized by loss of Ki67⁺ compartments and OlfM4 expression.

FIG. 2: Wnt target genes are downstream of Lgr4/5

Concomitant deletion of Lgr4 and Lgr5 in Lgr4^{fl/fl}-Lgr5^{fl/fl} mice resulted in the significant downregulation of 307 unique genes one day post deletion (Table 2). The intestinal Wnt signature can be revealed by two opposing experiments: deletion of Apc in vivo results in upregulation of a Wnt target signature⁹⁸, whereas Rspo-1 withdrawal from intestinal organoids⁹⁹ results in the immediate downregulation of Wnt target genes.

FIG. 2A: Heatmap of the log 2 ratio of Apc^{fl/fl} mice vs. wt mice for the 307 Lgr4/5 genes 3 days after deletion of Apc (ratios taken from Van Es et al. 2005¹⁰⁰).

FIG. 2B: Heatmap of the log 2 ratio of intestinal organoids 1 day after Rspo-1 withdrawal (−Rspo1) versus control organoids (+Rspo1) for the 307 Lgr4/5 gene set.

FIG. 2C: Gene Set Enrichment Analysis (GSEA). Genes are ranked according to their differential expression between Apc-deleted and wt mice (data from Sansom, O. J. et al. 2007⁹⁸). Black bars beneath the graph depict the rank positions of the 306 genes from the Lgr4/5 gene set. A highly significant enrichment of the 306 Lgr4/5 genes was detected towards the gene set upregulated 3 days after Apc deletion in vivo.

FIG. 2D: Genes are ranked according to their differential expression in intestinal organoids after Rspondin1 (Rspo-1) withdrawal versus control organoids. GSEA shows a highly significant enrichment of the 306 Lgr4/5 genes towards the genes downregulated after Rspo-1 withdrawal. ES: enrichment score, NES normalized ES, FDR: false discovery rate.

FIG. 3: Lgr4 and -5 proteins interact with Frizzled/Lrp complexes and Rspo-1 as assessed by mass spectrometry.

Left panel (Exps 1-7): HEK293T cells were transiently transfected, and LS174T cells were stably, transfected with the indicated tagged proteins. Right panel (Exps 8-9): HEK293T cells were incubated with the indicated tagged soluble proteins at 1 μg/ml. Sequential immunoprecipitation was performed by anti-FLAG and anti-HA antibodies and the associated complexes were analysed by mass spectrometry. Number of assigned spectra, number of unique peptides and the MASCOT Protein Score are given for selected identified proteins.

FIG. 4: Direct physical interaction of Rspo-1 with Lgr4/5/6 exodomains

FIG. 4A: Soluble Rspo-1 binds to Lgr4, Lgr5 and Lgr6. Top panel: HEK293T cells were transiently transfected (“tx”) with FLAG/HA tagged versions of Lgr4, Lgr5 or Lgr6, and with tagged Frizzled5 (Frz5) or mock as control, incubated with a conditioned medium containing an Rspo1-Fc fusion protein at ˜1 μg/ml where indicated. Cells were washed, lysed and Rspo1-Fc was immunoprecipitated with proteinG beads. Western blotting for the FLAG tag revealed binding of soluble Rspo1-Fc to Lgr4, Lgr5 and Lgr6, but not to Frz5 or mock-transfected cells. Bottom panel: Western blotting of cell lysates for the FLAG tag reveals the presence of the transfected, tagged proteins.

FIG. 4B: Rspo-1-Lgr5 interaction visualized by Surface Plasmon Resonance array imaging. Anti-FLAG antibody spotted on the sensor chip, readily captured Rspo-1-FH (left). The extracellular domain of Lgr5 was expressed as a human IgFc fusion protein (Lgr5-exo-Fc). After a wash, Lgr5-exo-Fc bound to Rspo-1-FH. After regeneration with low pH, a Noggin-Fc fusion protein served as negative control (right). Both Lgr5-exo-Fc and Noggin-Fc could be captured on goat anti-human IgG spotted as a control.

FIG. 4C: Epitope mapping of anti-human Lgr5 antibodies. Ab1 recognizes the cystein-rich linker (CRL)-region in isolation. Mapping of all other epitopes was performed by human-mouse hybrid Lgr5 clones (white bars indicate regions of human origin), allowing mapping of the human-specific antibodies as indicated. LRR: leucine-rich repeat region.

FIG. 4D: N-terminal-binding antibodies block Lgr5-exo-Fc/Rspo-1 interaction. The Lgr5 fusion protein was pre-incubated with the individual monoclonal antibodies followed by incubation with Rspo-1-FH bound to anti-FLAG beads. Blocking is visualized as loss of Lgr5-exo-Fc bound to the Rspo-1-coated beads by Western blotting for the Fc portion.

FIG. 5: Lgr4 is essential for transmitting Rspo-1 signals but dispensable for transmitting Wnt3A signals.

FIG. 5A: Downregulation of Lgr4 in HEK293T cells by transient transfection of three siRNAs (A, B, C) targeting the 3′UTR of Lgr4 or a scrambled siRNA (I). Expression assessed by Northern blotting three days post-transfection (top). Ethidiumbromide-stained gel shows equal loading as judged by ribosomal RNA bands (bottom).

FIG. 5B: TOPflash Wnt reporter assays. HEK293T cells were transfected with three different siRNAs (A, B, C) targeting the 3′UTR of Lgr4, or a non-targeting control siRNA (I). Three days later, the cells were transfected with TOPflash Wnt reporters +/−5 ng of the indicated human Lgr4, Lgr5 or Lgr6 rescue constructs and incubated with Wnt3A and Rspo-1 as indicated. Wnt3A readily induced TOPflash reporter activity without showing effects of removal of Lgr4. Rspondin potentiated the Wnt3A response, but this effect was sensitive to removal of Lgr4. Partial to virtually complete rescue was obtained with Lgr4, Lgr5, and Lgr6 rescue constructs.

FIG. 6: Rspo-1-Lgr5 interaction K_{D} determination by Surface Plasmon Resonance array imaging. Anti-FLAG antibody spotted on the sensor chip, readily captured Rspo-1-FH. Results are given as overlay plot of three injection series of first huRSPO1-FH followed by huLGR5-Fc. Undiluted huRSPO1-FH at 1 μg/ml is injected after 300 s baseline. Saturation of the anti-Flag spot is within 2 minutes and a wash with running buffer resulted in a stable second baseline that shifted 28 millidegree which corresponds to a huRSPO1-FH ligand density of ˜280 pg/mm². Three injections of huLGR5-Fc of 45 nM, 23 nM and 11 nM at 900 s resulted in a specific binding of LGR5-Fc on top of the RSPO1-FH and the exponentials were fitted with a discrete 1:1 interaction model. The residual plot shows a +2 till −2 mdegree experimental minus fit curve (perfect fit will give instrumental noise only) and the apparent KD of 3 nM is an average value calculated from a distribution of affinities and deviation to the 1:1 interaction model.

FIG. 7 Direct physical interaction of Rspondin1 with Lgr4/5/6 exodomains

FIG. 7A: Rspondin1 binds to Lgr4, Lgr5 and Lgr6. Top panel: HEK293T cells transfected (“tx”) with tagged versions of the indicated proteins, incubated with Rspo1-Fc fusion protein at ˜1 μg/ml. Cells were washed, lysed and Rspo1-Fc was immunoprecipitated with proteinG beads. Top: Western blotting for FLAG revealed specific binding to Lgr4, Lgr5 and Lgr6. Bottom: Input of tagged proteins.

FIG. 8. Lgr4 is essential for transmitting Rspondin1 signals but dispensable for transmitting Wnt3A signals.

FIG. 8A: Expression and downregulation of Lgr4 in HEK293T cells. Left: Northern blot for Lgr4 and Lgr5 on Ls174T and HEK293 cells as indicated. Right: Downregulation of

Lgr4 by three siRNAs (A, B, C) targeting the 3′ UTR of Lgr4, or a control siRNA (I). Expression assessed three days post-transfection (top). Ethidiumbromide-stained gel as loading control (bottom).

FIG. 8C: TOPflash Wnt reporter assays. As in FIG. 5B. Rescue performed with wt Lgr5 (ERG) or mutant Lgr5 (ENG) constructs. Rescue was obtained both with wt and mutant Lgr5 constructs.

FIG. 9. Rescue of Lgr4/5 deletion in cultured crypt organoids by Wnt signals.

FIG. 9A: Organoids established under standard conditions¹⁵ from Lgr4^{fx/fx} Lgr5^{f/fl} Villin-CreERT2 mice³⁶ or from control Villin-CreERT2 mice. Tamoxifen treatment leads to death (asterisks) of mutant organoids but not of controls. This is overcome by addition of CHIR99021 at 5 μM.

FIG. 9B: When CHIR99021 is subsequently withdrawn from Lgr4/5 deleted organoids after passage, they immediately undergo apoptosis.

FIG. 9C: Organoids established from Lgr4^{fx/fx} Lgr5^{f/fl} Villin-CreERT2 mice or from control Villin-CreERT2 mice are infected with Wnt3-expressing retrovirus which turns organoids into growing, rounded cysts³⁴, or with control retrovirus. Subsequent deletion of Lgr4 and -5 has no effect on Wnt3-expressing organoids but leads to death of control organoids.

FIG. 10. Early and late lineage tracing in Lgr4^{GFR-ires-CreERT2}×R26R-LacZ Cre reporter mice. Generation of the Lgr4 allele was identical to that of the Lgr5 and Lgr6 alleles. Lineage tracing was performed and analyzed at 2 days post tamoxifen induction for scoring transit amplifying cell tracing and at 395 days post-induction for scoring stem cells. While most early tracing events resulted in marking single cells in crypts and villi (top panel), which disappeared over the next few days, significant numbers of stem cell tracing events were also noted as “blue ribbons” (middle panel: whole mount; lower panel: paraffin section histology. LacZ tracing in blue, tissue counter-stained in red). No tracing events were observed without tamoxifen induction.

FIG. 11 Design of Lgr5^{fl} allele.

FIG. 11A: Black triangles indicate the inserted loxp sites. PMCNeo was removed by cre-mediated deletion in the germline.

FIG. 11B: PAS stain 5 days after gene deletion shows no effects on differentiated compartments. PAS staining of small intestine of mice of the indicated genotype, 5 days after gene deletion. Dark counterstain. Goblet cells: dyed body on villi; enterocyte: dyed apical membrane; Paneth cells: dyed cell body at crypt bottoms. Top panel is indistinguishable from wt intestine. Bottom panel: Arrows point to Paneth cell “nests” that remain after disappearance of proliferative crypt cells.

FIG. 12 Stem cells are still present one day after deletion of Lgr4 and Lgr5.

Top/Middle: Ki57 stain for proliferative cells; light counter-stain. One day after deletion of Lgr4 and Lgr5, mutant intestine (middle) is undistinguishable from wt intestine (top). Arrows indicate proliferative stem cells.

Bottom: Representative EM images of crypt base of an adult mouse homozygous for Lgr4^{fx} and Lgr5^{fl}, and carrying the Ah-Cre transgene analyzed one day (left) and five days (right) post-B-naphtoflavone-induced deletion. Stem cells (S) are physically present at one day between the Paneth cells, but absent at day 5. Note extensive RER and secretory granules in Paneth cells (P)

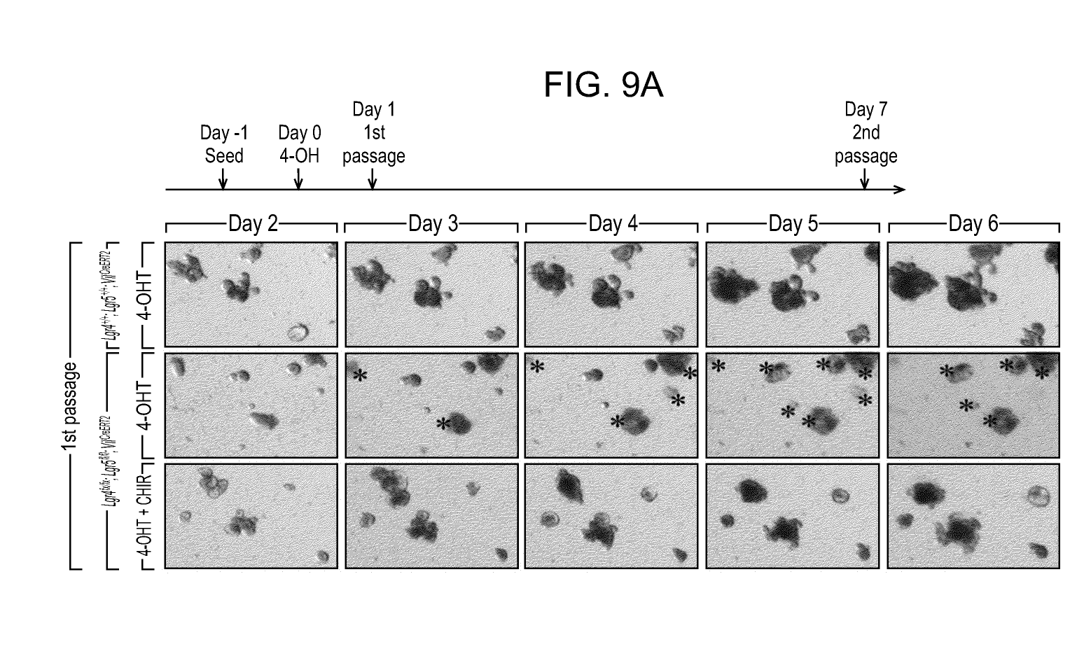

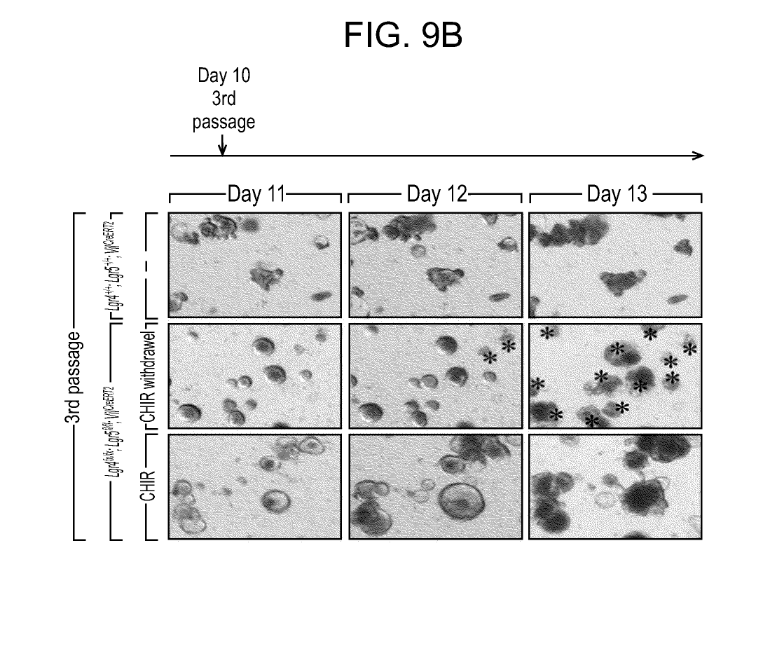

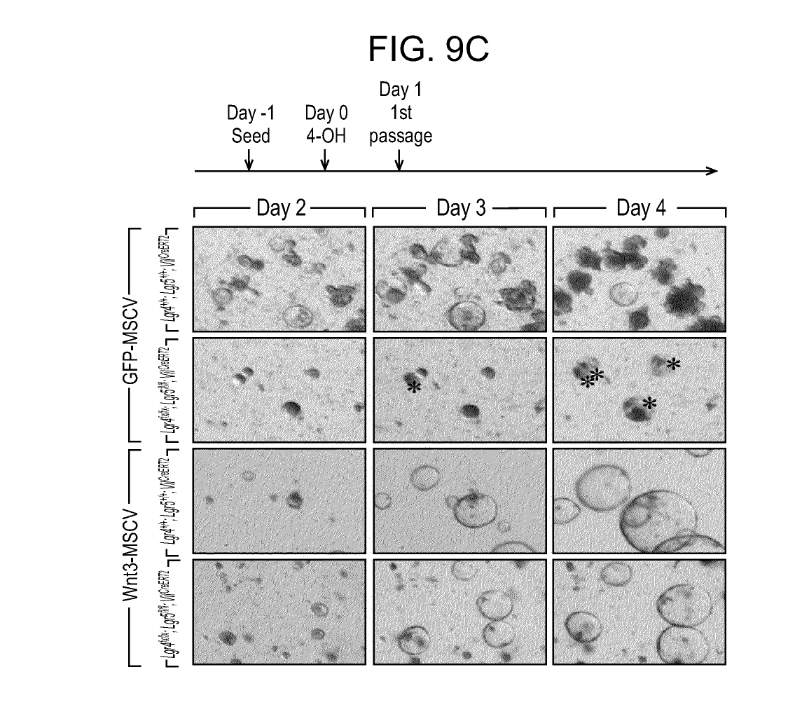

FIG. 13 All Rspondins bind to Lgr4, Lgr5 and Lgr6. Rspondin1-4 were produced as IgFc fusion proteins and subjected to the assay depicted in FIG. 9A. As is evident, all four Rspondins bound specifically to FH-tagged versions of human Lgr4, -5 and -6 transfected into HEK293T cells, but not to control Lgr7. Top: input of the four Lgr5 proteins. Bottom: Pull-down with Rspo1-Fc protein. Only the full-length Lgr5 protein binds Rspo1.

FIG. 14 N-terminal domains of Lgr5 are required for Rspo1 binding.

FIG. 14A: Same assay strategy as in FIG. 15. FL: full-length. ΔN: Deletion of the extreme N-terminus of Lgr5 (amino acids: 28-50). ΔLRR1: deletion of the first LRR domain from Lgr5 (amino acids: 51-75), ΔLRR1-17: Deletion of N-terminus and entire LRR region. Only full-length protein binds to Rspo1.

FIG. 14B: Same as in A) but incubations with Rspo1 were performed after cell lysis.

FIG. 15: Rspondin1-Lgr5 interaction KD determination by Surface Plasmon Resonance array imaging. Plot of the apparent kd and KD calculated values using a 1:1 binding model from serial injections of Lgr5-Fc to the array of RSPO-1 captured spots as measured in the IBIS MX96 instrument of IBIS Technologies (Enschede, The Netherlands). In IBIS-SPRint software (version 2) an exponential fitting routine y=a*ebx was used for extrapolating the values of KD and kd at Rmax=0 (arrows). The coëfficient of the exponential a (y-intercept) resulted in kd=1.55*10-4 s-1, KD=3.07*10-9 M respectively (ka=5.04*104 M-1s-1) for binding of the Lgr5-Fc to RSpo-1-FH.

Method: IBIS-SPRinT 2.0. An array of anti-Flag spots with decreasing densities was printed on a preactivated sensor surface (IBIS Technologies, Enschede, the Netherlands) using a continuous flow microspotter (CFM) (Wasatch Microfluidics, Utah, US). Undiluted RSPO-1-FH supernatant was exposed to the array and each anti-Flag spot captured a decreasing density of RSPO-1-FH as shown in the sensorgram of figure X. After measuring a new baseline for each spot the LGR5-Fc was injected and the sensorgram of each spot was recorded simultaneously. Rmax values were correlated to ligand (RSPO-1) densities.

The apparent rate- and affinity constant kd, ka and KD may change significantly, if rebinding effects, biphasic behavior and/or steric hindrance of the immobilized/captured molecules occurs at the sensor chip surface. A common strategy is to load the ligand at a very low density to mimic homogeneous binding in solution, preferably just above the limit of detection of the instrument. However, also at low ligand densities, the negative influence of rebinding etc. to the rate- and affinity constants kd, ka and KD may occur. Here we apply a method for the determination of kd, ka and KD, which uses all biomolecular interaction data of LGR5 to RSPO-1 responses of the different spots, resulting in a single rate- and affinity constant kd, ka and KD at Rmax=0 by exponential fitting the apparent kd and KD values to Rmax=0. Theoretically, this means that the affinity constant of a single ligand molecule to its single analyte molecule is determined in the limit to zero response.

FIG. 16 Dose response curve of Rspondin in Wnt reporter assay.

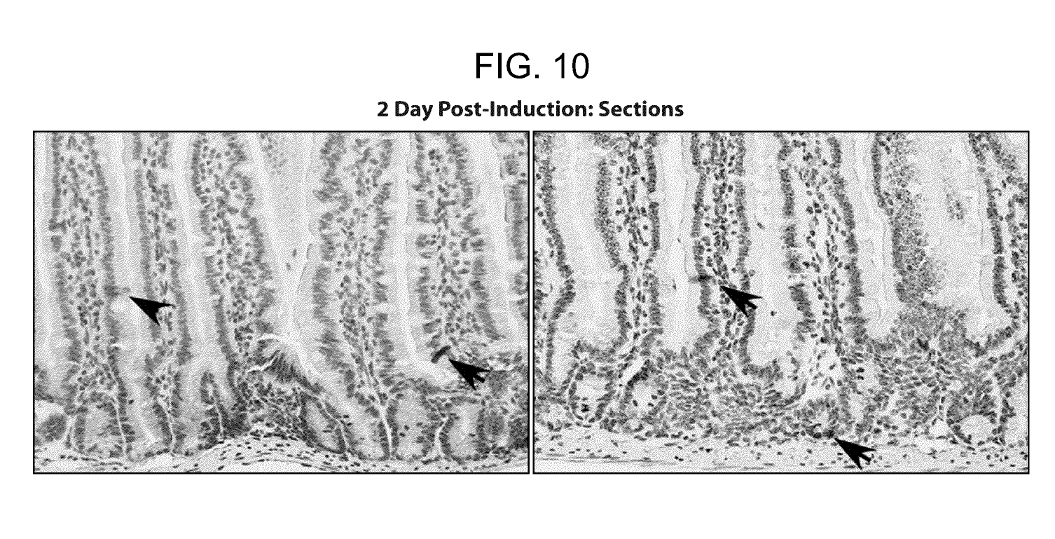

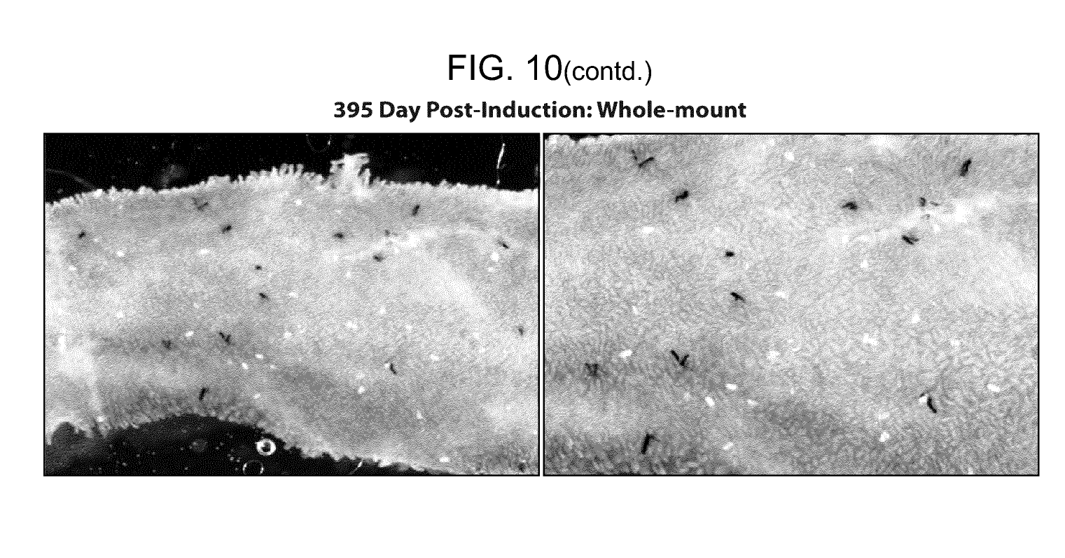

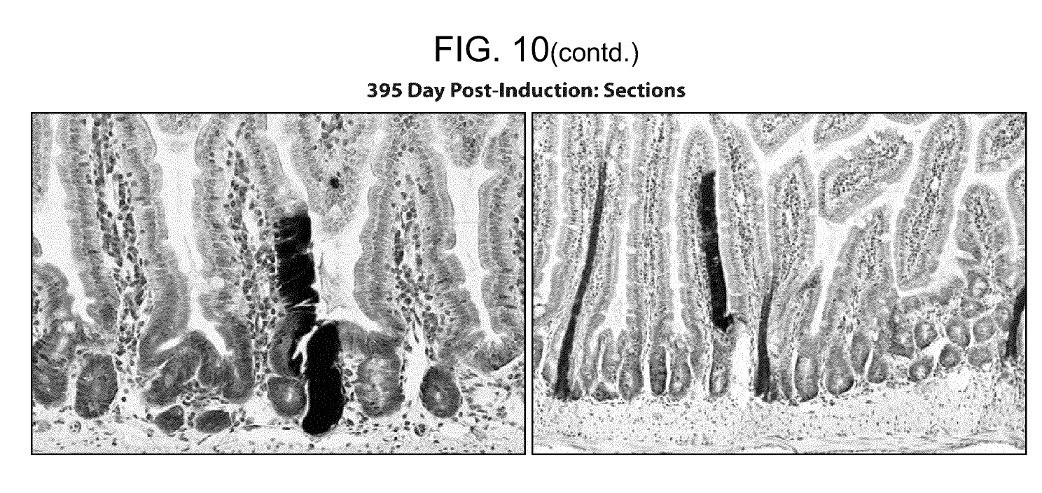

Experiment performed in HEK293T cells as in FIG. 10, with constant amount of Wnt and increasing concentrations of Rspondin1. Transfections were done in duplicate; both values are given.

FIG. 17 Only Lgr4, 5, or 6 can rescue Rspondin responsiveness.

A) Assay performed as in FIG. 10B. No rescue was obtained with Lgr1, Lgr7, or Lgr8.

FIG. 18. Rescue of Lgr4/5 deletion in cultured crypt organoids by retrovirally expressed Lgr5.

Representative images of organoids established from Lgr4^{fx/fx} Lgr5^{f/fl} Villin-CreERT2 mice are infected with retrovirus expressing GFP (top), Lgr5 (not shown) and Lgr5-ENG (bottom). Both Lgr5-expressing retroviruses protected against subsequent deletion of Lgr4 and -5, implying that G protein signaling is not essential for Lgr signaling. Arrows indicate growth of rescued organoids.

FIG. 19 Lgr5 binds to the Furin like domain of RSp4

19A: shows the immunoprecipitation of flag tagged human RSpo4 proteins. hRSPO4 WT (Full length); hRSP4 daa (deletion of the basic amino acid domain); hRSPO4 dtht+ aa (deletion of the thrombospondin domain and Basic domain. These proteins are used to precipitate either hLGR5 (Fc tagged) or DKK (flag tagged). It was found that Lgr5 but not Dkk binds to hRSPO4 and its deletion that include only the furin domain. This indicates that Lgr5 binds to the furin domain.

19B: A flag tag Western blot shows equal expression of all RSPO proteins.

19C: A diagram showing the lanes used in the Western blot.

FIG. 20: Activation of B-Cat/TCF TOP promoter by Wnt, RSpo and LGR5 antibodies.

FIG. 20A: HEK293T cells stably transfected with LGR5 are transfected with the TOP reporter and Renilla reporter constructs. One day later the cells are stimulated with: 1) Wnt3A condition medium (Produced in L-Cells and added at ⅓ of the total medium volume), 2) RSpo His tagged purified on a nickel column from 293T cells added at 1 ug/ml, 3) Anti LGR5Rat monoclonal antibodies 1D9, 4D11, 8F2 prot A purified at 1 ug/ml. Luciferase activity is determined 24 h after stimulation. The luciferase counts represent the level of activation of the Wnt pathway compared to the control.

FIG. 20B: HEK293T cells were seeded into 96-well plates in DMEM/10% FCS at a density of 10⁴ cells/well and in triplicate transfected (PEI: Polyethylenimine, linear, MW-25,000) with 10 ng TOP or FOP luciferase (Ref 1), 1 ng TK Renilla, 10 ng of pcDNA-based hLgr5-Flag, and 80 ng empty vector DNA. After 24 hrs, medium was replaced for 50% fresh DMEM/10% FCS, and 50% Wnt3a-conditioned medium or control conditioned medium. At the same time point, purified human Rspondin1 (stock solution of 100 μg/ml in PBS) was added at a final concentration of 1 μg/ml and ProtA-purified (stock solution of 1 mg/ml in PBS) Lgr5-specific antibodies (1D9 and 8F2) at three different concentrations. At 72 hrs results were measured using a dual luciferase assay kit (Promega USA). The luciferase counts represent the level of activation of the Wnt pathway compared to the control.

FIG. 21 Graph showing effect of anti-Rspo3 mAb on Wnt activity Blocking of RSpondin-induced enhancement of Wnt signaling by rat anti h/m Rspondin3 antibody;

HEK293T cells were transfected with Wnt pathway-specific (TOP) luciferase reporter in combination with a TK driven Renilla reporter. After 24 hrs cells were incubated for 24 hrs. with control conditioned medium (CM), Wnt3a CM, or combinations of Wnt3a+Rspondin-CM. The relative volume proportion (%) of Rspondin CM is indicated. A rat monoclonal antibody (R&D systems, clone 400403), recognizing both human and mouse Rspondin3, was added to a final concentration of 2.5 μg/ml. Wnt pathway activity was quantitated by comparing averages of triplicate Renilla-normalized TOP luciferase counts of cells receiving stimuli, to un-stimulated cells.

FIG. 22 Isolated furin domains enhance Wnt signaling to a greater extent than full-length Rspondin

The graph shows TOP luciferase counts relative to control (y-axis) for full length Rspo1, compared to truncated fragments of Rspo1, as described below. Fragment FUR1/2+Thr is represented by SEQ ID NO: 139 and fragment FUR1/2 is represented by SEQ ID NO: 140. The numbers represent the lanes on the x-axis.

| 1 = | ctr medium | | |
|---|---|---|---|
| 2 = | Wnt3a | | |
| 3 = | Wnt3a + Rspo1Fc-conditioned medium | | |
| 5 = | Wnt3a FL Rspo1-HIS | 62 | nM |
| 6 = | | 30 | nM |
| 7 = | | 15 | nM |
| 8 = | | 7 | nM |
| 9 = | | 3 | nM |
| 10 = | | 1.5 | nM |
| 5 = | Wnt3a FUR1/2 + Thr-HIS Peng | 62 | nM |
| 6 = | | 30 | nM |
| 7 = | | 15 | nM |
| 8 = | | 7 | nM |
| 9 = | | 3 | nM |
| 10 = | | 1.5 | nM |
| 5 = | Wnt3a FUR1/2-HIS | 62 | nM |
| 6 = | | 30 | nM |
| 7 = | | 15 | nM |
| 8 = | | 7 | nM |
| 9 = | | 3 | nM |
| 10 = | | 1.5 | nM |

FIG. 23 Antibody 1D9 can be used as an Rspondin mimic in culture media

This figure shows mouse colon organoids grown in Sato medium (Sato et al. Nature 459, 262-5, 2009). Left column show organoids grown in the presence of full length RSP1. Middle column shows the media with supplemented with the 1D9 antibody instead of RSP1. Right column shows organoids grown without RSP1 or 1D9.

FIG. 24 Epcam-Ab-Rspondin1 furin domain fusion (a bispecific agonist targeting epithelial cells) is effective in culture media at lower concentrations than Rspondin1 alone.

This figure shows mouse colon organoids grown in Sato medium (Sato et al Nature 459, 262-5, 2009). Left column show organoids grown in the presence of full length RSP1. Middle column has the EpcamAb-RSP furin domain fusion at a concentration 50× lower than the full length RSP1 in the left column. The right column shows organoids without RSP1 or the fusion protein.

FIG. 25 Epcam-Ab-Rspondin1 furin domain fusion in a TOP/FOP assay

This graph shows the results of a TOP/FOP assay for the multi-targeting compound, EpcamAb-RSP furin domain fusion, compared to normal wild-type Rspondin1. The luciferase counts on the y-axis are a measure of Wnt pathway activation compared to a control. This figure shows that Epcam-Ab-Rspondin1 furin domain fusion mimics the activity of Rspondin for enhancing Wnt activation at a 16× lower concentration than Rspondin.

FIG. 26 1D9 antibody binds the CRL region of LGR5

This figure demonstrates that the 1D9 antibody binds the CRL region of LGR5. Purified proteins and antibodies were used. LGR5-543 represents the full exo domain of LGR5 (CRL+Leucine rich repeats). LGR5-478 represents the exo domain of LGR5 without the CRL region.

This graph represents the protein complexes separated by size on a gel filtration column. On the left are the proteins running of the column first (so the biggest complexes). From left to right we have a blue line with two peaks. The left peak represents the LGR5-543/RSp1/Antibody 1D9 complex. The next peak of the same line is the LGR-543/RSp complex (added in excess). The next separation represented by the second line from the left having only a single peak is the antibody alone (black line). Below this the separation of the LGR5-478 incubated with the Antibody. This purification shows that the antibody purifies with the Ab alone peak. The next peak on the same line is at the size of the Lgr5-RSp peak. This demonstrates that the Ab did not bind the complex (green line). Last the middle sized peak (amplitude) on the right represent the Lgr5/RSp complex. This line has a single peak (Red line).

FIG. 27 Lgr is expressed on the basolateral side of the membrane

The figures show fluorescent staining of his-tagged recombinant fusion Lgr5 (left) and Lgr4 (middle) compared to the control (right), in mouse colon organoids grown in Sato medium (Sato et al. Nature 459, 262-5, 2009). It can be seen that Lgr proteins are expressed on the basolateral (non-lumen) side of the membrane.

FIG. 28 TOP/FOP assay showing Lgr4 and Lgr5 antagonists

This experiment is done with the stable transfected HEK-Lgr5FlagHA or HEK-Lgr4FlagHA cells. All cell have been transfected with the Wnt activity reporter TOP/FOP. Wnt activity was tested with and without several Lgr5 and Lgr4 rat monoclonal antibodies.

28A: Lgr5 antibodies—From left to right: nothing added (control); Wnt only added; Wnt+RSp1; Wnt+RSpo+Antibody medium without antibody; Wnt+all antagonistic antibodies; far right-hand column shows Wnt+the agonistic antibody 1D9.

28B: Lgr4 antibodies—From left to right: nothing added (control); Wnt only added; Wnt+RSpo+Antibody medium without antibody; Wnt+all antagonistic antibodies;

### MODES FOR CARRYING OUT THE INVENTION

Various aspects and embodiments of the present invention will now be described in some detail. It will be appreciated that modification of detail may be made without departing from the invention.

### Example 1

### Lgr4/5/6 Family Members Reside in Frizzled-Lrp Complexes and Mediate Signaling by the Wnt Agonist Rspondin1

The role of Lgr proteins in stem cells was previously unknown. Similarly, whilst a number of contrasting explanations had been postulated, the exact mechanism by which Rspondin activates the Wnt/β-signalling was also unknown. In this first example, the inventors describe the surprising discovery that Lgr proteins, which are Wnt target genes, also play a role themselves in the Wnt signalling pathway and form part of the Wnt receptor complex. Furthermore, the inventors demonstrated that Rpsondin binds to Lgr proteins in the Wnt receptor complex. The inventors thus describe an unexpected new mechanism for Rspondin-mediated Wnt/β-catenin signalling activation. This provides new avenues for identifying agonists for the Wnt/β-catenin signalling pathway, which mimic the Rpsondin-Lgr interaction.

The adult stem cell marker Lgr5 and its relative Lgr4 are often co-expressed in Wnt-driven proliferative compartments. In summary, the inventors find that conditional deletion of the two genes in the gut impairs Wnt target gene expression and results in rapid demise of intestinal crypts, thus phenocopying Wnt pathway inhibition. Mass-spectrometry demonstrates that Lgr4 and Lgr5 associate with the Frizzled/Lrp Wnt receptor complex. Each of the four Rspondins, secreted Wnt pathway agonists, can bind to Lgr4, -5 and -6. In HEK293 cells, Rspondin1 enhances canonical Wnt signals initiated by Wnt3A. Removal of Lgr4 does not affect Wnt3A signaling, but abrogates the Rspondin1-mediated signal enhancement, a phenomenon rescued by re-expression of Lgr4, -5 or -6. Genetic deletion of Lgr4/5 in intestinal crypt cultures phenocopies withdrawal of Rspondin1 and can be rescued by Wnt pathway activation. Lgr5 homologs are facultative Wnt receptor components that mediate Wnt signal enhancement by soluble Rspondin proteins.

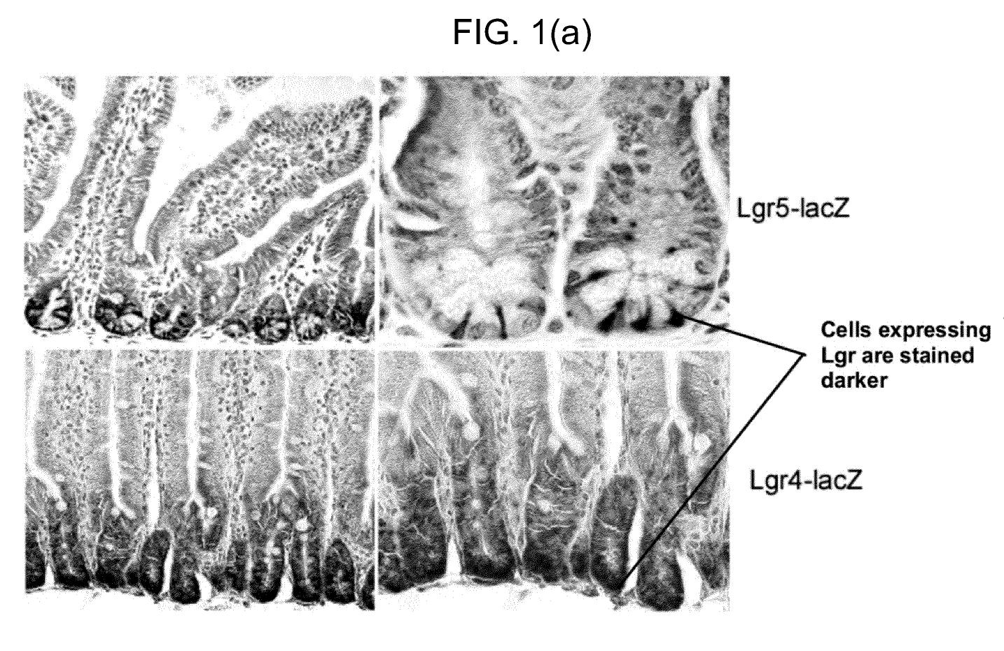

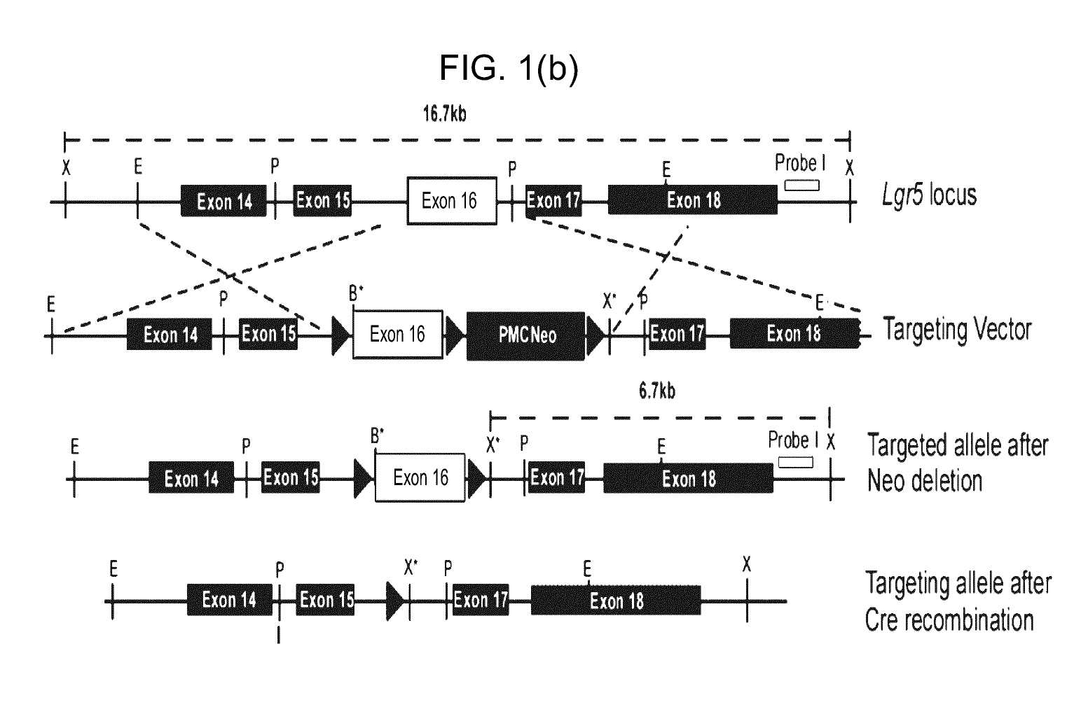

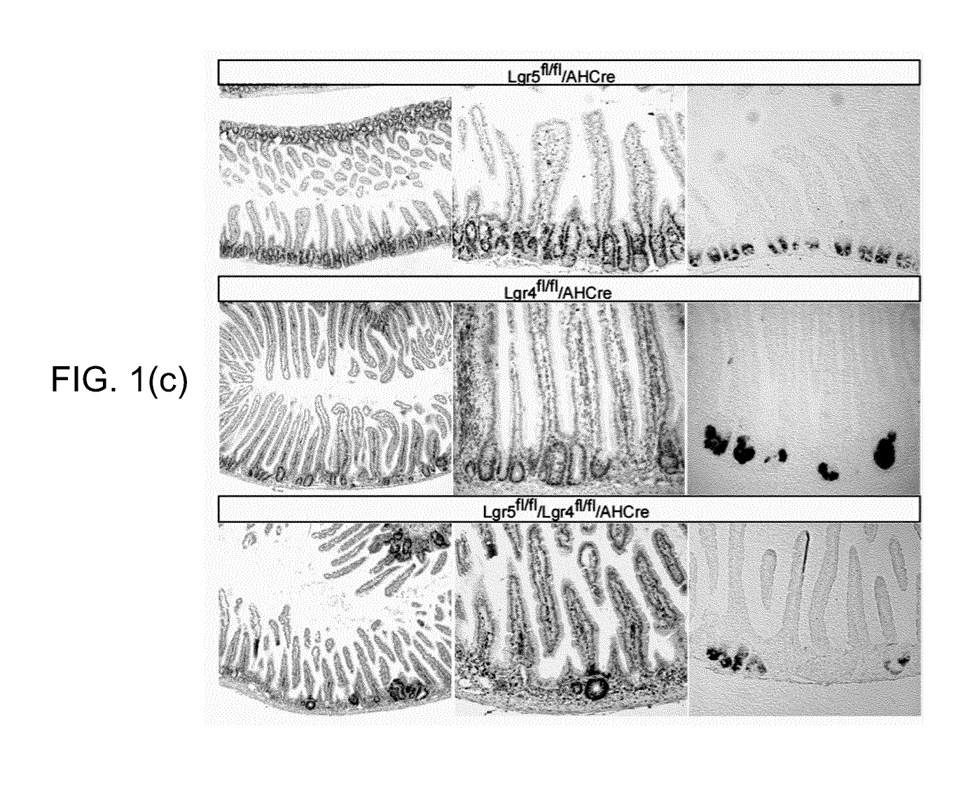

Lgr4, Lgr5 and Lgr6 encode orphan 7-Transmembrane receptors, that are close relatives of the receptors for the hormones FSH, LH and TSH¹⁰¹. It was previously unknown how the Lgr4-6 receptors signal. Lgr5 is a Wnt target gene which marks proliferative stem cells in several Wnt-dependent stem cell compartments, specifically the small intestine and colon¹⁰², the stomach¹⁰³, and the hair follicle¹⁰⁴. Lgr6 marks multipotent stem cells in the epidermis¹⁰⁵. The expression of Lgr4 is much broader¹⁰⁶, but the inventors noted that Lgr5 is co-expressed with Lgr4 in the stem cell compartments mentioned above. For instance, Lgr5 marks small intestinal stem cells at the base of crypts, while Lgr4 marks all crypt cells, including the Lgr5⁺ stem cells (FIG. 1A). Both, Lgr4 and Lgr5 null mutations are neonatal-lethal in mice^{107, 108}.

Lgr5 and Lgr4 are often co-expressed in Wnt-driven proliferative compartments. To address a potential function in crypts, the inventors generated the Lgr5^{fl} allele (FIG. 1B) in which exon 16 is flanked by loxp sites; its deletion causes a frame shift. This allele was crossed into a mouse strain carrying conditional Lgr4^{fx} alleles¹⁰⁸ and the gut-specific Ah-Cre transgene, which is inducible by β-naphtoflavone¹⁰⁹. Conditional deletion of Lgr5 alone in the intestinal epithelium of adult mice yielded no apparent phenotype. In particular, the Paneth cell phenotype reported previously in Lgr5 null neonatal mice¹¹⁰ could not be confirmed. Deletion of Lgr4 alone resulted in a deleterious effect on the proliferative cells in crypts, which typically became obvious from day 4-5 post-induction onwards. Crypt proliferation halted and many crypts disappeared. No direct effects were observed on the differentiated compartments of the villus. The combined deletion of Lgr4 and -5 enhanced the crypt phenotype as judged by the cell proliferation marker Ki67 and the stem cell marker Olfm4¹¹¹. FIG. 1C depicts typical results obtained at day 5 post-induction. Over the next few days, villi typically shortened because of the halted cell production in crypts and eventually the phenotype was not compatible with life.

Two signalling pathways, Wnt^{112, 113} and Notch¹¹⁴, are crucial for the maintenance of adult crypt proliferation. To determine whether Lgr4/5 genes may be involved in these signalling pathways, the inventors performed differential gene expression analysis by microarray on small intestinal crypts isolated from Lgr4/5 knock-out mice. To address immediate changes in gene expression, the gene expression analysis performed on day 1-post-deletion of Lgr4 and -5, before any histological changes are apparent. Simultaneous deletion of Lgr4 and Lgr5 resulted in the significant downregulation of 307 genes in two separate experiments (gene identities are shown in Table 2).

Table 2 shows 306 unique genes significantly downregulated in Lgr4/5 double knock-out mice one day after deletion in two biological replicates performed as a dye swap experiment, resulting in 4 individual arrays:

| | Probe | Biological Replicate 2 | | Biological Replicate 2 | | | |
|---|---|---|---|---|---|---|---|
| | Name | Dye swap 1 | Dye swap 2 | Dye swap 1 | Dye swap 2 | Avg | GeneName |
| 1 | A_52_P87997 | 2.13 | 1.39 | 3.90 | 3.90 | 2.83 | Gsdmc2 |
| 2 | A_51_P150489 | 1.76 | 2.21 | 3.44 | 3.69 | 2.77 | Gsdmc1 |
| 3 | A_51_P521155 | 2.25 | 2.11 | 3.42 | 3.25 | 2.76 | 1700124P09Rik |
| 4 | A_52_P498798 | 2.36 | 2.10 | 2.99 | 3.09 | 2.64 | Gsdmc3 |
| 5 | A_51_P312336 | 2.57 | 2.97 | 2.45 | 2.44 | 2.61 | Slc14a1 |
| 6 | A_52_P25357 | 3.06 | 3.06 | 1.98 | 1.82 | 2.48 | NAP027049-1 |
| 7 | A_52_P387458 | 1.65 | 1.55 | 3.29 | 3.14 | 2.41 | 1810035L17Rik |
| 8 | A_51_P337308 | 0.85 | 2.61 | 2.78 | 3.30 | 2.39 | Saa3 |
| 9 | A_51_P179878 | 3.49 | 2.69 | 1.90 | 1.42 | 2.38 | 2900019G14Rik |
| 10 | A_51_P501840 | 2.10 | 2.75 | 2.10 | 1.94 | 2.22 | Dnajb3 |
| 11 | A_52_P251690 | 1.64 | 2.40 | 2.34 | 2.48 | 2.22 | Gvin1 |
| 12 | A_51_P267700 | 2.52 | 2.65 | 1.89 | 1.81 | 2.22 | 1190003M12Rik |
| 13 | A_52_P566963 | 0.93 | 1.67 | 2.90 | 3.12 | 2.15 | Msi1 |
| 14 | A_51_P515605 | 1.31 | 2.39 | 2.51 | 2.34 | 2.14 | Col3a1 |
| 15 | A_52_P67463 | 1.51 | 0.62 | 3.51 | 2.87 | 2.13 | 1810026B05Rik |
| 16 | A_52_P1016836 | 1.56 | 1.96 | 2.50 | 2.45 | 2.12 | ENSMUST00000061000 |
| 17 | A_52_P403157 | 1.86 | 2.83 | 1.90 | 1.75 | 2.09 | Sorbs2 |
| 18 | A_52_P503387 | 0.69 | 1.32 | 3.15 | 3.16 | 2.08 | Trp53inp1 |
| 19 | A_51_P225493 | 1.66 | 1.09 | 2.91 | 2.55 | 2.05 | 1700016C15Rik |
| 20 | A_51_P117952 | 1.15 | 1.32 | 3.01 | 2.71 | 2.05 | Hspa1a |
| 21 | A_51_P285097 | 0.98 | 2.15 | 2.20 | 2.84 | 2.04 | Wdr38 |
| 22 | A_51_P218091 | 2.24 | 2.80 | 1.53 | 1.48 | 2.01 | Lgr5 |
| 23 | A_52_P488919 | 1.34 | 1.72 | 2.60 | 2.32 | 1.99 | Zbtb26 |
| 24 | A_51_P264685 | 0.59 | 1.38 | 3.02 | 2.97 | 1.99 | Whrn |
| 25 | A_51_P347547 | 1.30 | 1.51 | 2.50 | 2.64 | 1.99 | Klf10 |
| 26 | A_51_P450752 | 1.41 | 3.61 | 1.34 | 1.57 | 1.98 | Pla2g4b |
| 27 | A_52_P522166 | 0.63 | 1.06 | 3.03 | 3.17 | 1.97 | TC1654900 |
| 28 | A_52_P134195 | 1.39 | 3.83 | 1.30 | 1.28 | 1.95 | Ceacam10 |
| 29 | A_52_P668812 | 0.85 | 1.23 | 2.66 | 3.03 | 1.94 | Sgol2 |
| 30 | A_51_P182303 | 1.36 | 2.26 | 2.06 | 2.06 | 1.93 | Col1a2 |
| 31 | A_51_P130544 | 1.34 | 0.95 | 2.79 | 2.47 | 1.89 | Gas5 |
| 32 | A_52_P828218 | 1.78 | 2.36 | 1.44 | 1.94 | 1.88 | Olfm4 |
| 33 | A_52_P195772 | 1.70 | 1.68 | 1.96 | 2.19 | 1.88 | Rbmx |
| 34 | A_52_P682465 | 1.84 | 1.17 | 2.39 | 2.07 | 1.87 | Rps28 |
| 35 | A_52_P190744 | 0.94 | 1.96 | 2.18 | 2.37 | 1.87 | Rnpc3 |
| 36 | A_51_P507602 | 1.02 | 2.24 | 2.06 | 2.15 | 1.86 | Clca4 |
| 37 | A_52_P386075 | 1.19 | 1.19 | 2.36 | 2.67 | 1.85 | ENSMUSG00000057445 |
| 38 | A_51_P480709 | 1.39 | 1.61 | 2.10 | 2.28 | 1.84 | Ywhaz |
| 39 | A_52_P460584 | 1.14 | 2.45 | 1.91 | 1.81 | 1.83 | Tnfrsf25 |
| 40 | A_52_P367621 | 1.25 | 1.28 | 1.82 | 2.80 | 1.79 | Arid5b |
| 41 | A_51_P478486 | 0.80 | 0.73 | 2.32 | 3.18 | 1.76 | Fus |
| 42 | A_51_P185660 | 1.99 | 1.95 | 1.82 | 1.26 | 1.75 | Ccl9 |
| 43 | A_52_P222230 | 1.50 | 1.54 | 1.80 | 2.16 | 1.75 | AK157581 |
| 44 | A_52_P1052476 | 1.25 | 1.71 | 2.01 | 2.02 | 1.75 | Bub3 |
| 45 | A_51_P447189 | 1.21 | 1.15 | 2.29 | 2.34 | 1.75 | 1700030C10Rik |
| 46 | A_52_P145433 | 1.64 | 0.93 | 2.53 | 1.86 | 1.74 | D7Ertd715e |
| 47 | A_51_P324161 | 0.67 | 1.17 | 2.18 | 2.84 | 1.72 | Ankrd26 |
| 48 | A_52_P780821 | 1.25 | 0.77 | 2.20 | 2.60 | 1.71 | Msi2 |
| 49 | A_51_P285413 | 1.00 | 1.98 | 1.58 | 2.20 | 1.69 | Rbbp6 |
| 50 | A_52_P540219 | 1.06 | 2.29 | 1.48 | 1.91 | 1.69 | Timp2 |
| 51 | A_52_P211418 | 0.80 | 0.91 | 2.11 | 2.90 | 1.68 | 6030408C04Rik |
| 52 | A_52_P328825 | 2.24 | 2.55 | 0.92 | 0.90 | 1.65 | Wdfy1 |
| 53 | A_52_P529790 | 1.88 | 2.07 | 1.24 | 1.43 | 1.65 | Hisppd2a |
| 54 | A_52_P100002 | 1.32 | 0.92 | 2.41 | 1.97 | 1.65 | Rps24 |
| 55 | A_51_P460391 | 1.38 | 1.38 | 2.04 | 1.75 | 1.64 | Defcr21 |
| 56 | A_52_P355004 | 1.87 | 2.80 | 1.16 | 0.72 | 1.64 | Cttnbp2 |
| 57 | A_52_P419879 | 0.68 | 1.81 | 1.55 | 2.50 | 1.63 | TC1695874 |
| 58 | A_52_P309022 | 1.53 | 1.17 | 1.89 | 1.94 | 1.63 | Dach1 |
| 59 | A_51_P162984 | 1.83 | 0.99 | 1.92 | 1.78 | 1.63 | Prm1 |
| 60 | A_51_P129464 | 1.04 | 1.37 | 2.01 | 2.09 | 1.63 | Scd2 |
| 61 | A_52_P522157 | 0.76 | 0.68 | 2.63 | 2.41 | 1.62 | 2810026P18Rik |
| 62 | A_52_P214630 | 0.92 | 1.41 | 2.24 | 1.91 | 1.62 | Sox9 |
| 63 | A_52_P276727 | 1.74 | 1.54 | 1.72 | 1.46 | 1.62 | Lgr4 |
| 64 | A_52_P14778 | 1.23 | 1.81 | 1.67 | 1.73 | 1.61 | Piwil4 |
| 65 | A_51_P275527 | 1.12 | 1.89 | 1.63 | 1.79 | 1.61 | Slc12a2 |
| 66 | A_52_P1133481 | 1.05 | 1.96 | 1.85 | 1.56 | 1.61 | BF149456 |
| 67 | A_51_P508510 | 0.93 | 1.94 | 1.56 | 1.97 | 1.60 | Notch1 |
| 68 | A_52_P483885 | 1.22 | 1.58 | 1.92 | 1.67 | 1.60 | 4930455C21Rik |
| 69 | A_52_P679105 | 1.96 | 1.66 | 1.60 | 1.14 | 1.59 | Prss23 |
| 70 | A_51_P101719 | 0.70 | 0.82 | 2.28 | 2.56 | 1.59 | Ttc14 |
| 71 | A_52_P431615 | 1.32 | 1.98 | 1.23 | 1.81 | 1.58 | Gm1966 |
| 72 | A_51_P281326 | 1.28 | 1.87 | 1.54 | 1.57 | 1.57 | Clasp2 |
| 73 | A_52_P659448 | 1.88 | 1.11 | 1.44 | 1.83 | 1.56 | 91304300000 |
| 74 | A_52_P510119 | 0.89 | 0.79 | 2.09 | 2.45 | 1.55 | Pgm2l1 |
| 75 | A_51_P375543 | 1.28 | 1.18 | 1.67 | 2.08 | 1.55 | Myb |
| 76 | A_52_P407007 | 1.22 | 1.15 | 1.57 | 2.26 | 1.55 | 4632415L05Rik |
| 77 | A_52_P381009 | 0.78 | 0.99 | 1.99 | 2.45 | 1.55 | Tia1 |
| 78 | A_52_P306845 | 0.75 | 1.50 | 2.11 | 1.83 | 1.55 | Cav1 |
| 79 | A_51_P114287 | 1.02 | 0.80 | 2.18 | 2.16 | 1.54 | Cpsf6 |
| 80 | A_51_P346715 | 0.88 | 1.30 | 1.89 | 2.09 | 1.54 | D4Wsu53e |
| 81 | A_51_P463765 | 1.11 | 2.19 | 1.43 | 1.42 | 1.54 | Timp3 |
| 82 | A_52_P16136 | 1.07 | 0.75 | 2.10 | 2.21 | 1.53 | D10Bwg1070e |
| 83 | A_52_P35534 | 2.24 | 2.51 | 0.78 | 0.59 | 1.53 | E230029C05Rik |
| 84 | A_51_P222936 | 0.86 | 2.38 | 1.45 | 1.42 | 1.53 | Fmnl2 |
| 85 | A_52_P119039 | 0.93 | 1.24 | 1.75 | 2.14 | 1.52 | Hmgcs1 |
| 86 | A_51_P398525 | 1.37 | 2.70 | 0.97 | 1.02 | 1.51 | Fn3k |
| 87 | A_52_P71261 | 0.82 | 0.74 | 2.27 | 2.21 | 1.51 | TC1615264 |
| 88 | A_51_P172688 | 1.25 | 1.26 | 1.78 | 1.75 | 1.51 | Stard3nl |
| 89 | A_51_P201254 | 0.59 | 0.94 | 2.16 | 2.30 | 1.50 | AK047739 |
| 90 | A_52_P465809 | 0.92 | 0.94 | 1.91 | 2.18 | 1.49 | Pwwp2a |
| 91 | A_51_P501757 | 0.82 | 1.64 | 1.68 | 1.78 | 1.48 | Rgmb |
| 92 | A_52_P566487 | 0.61 | 1.14 | 2.19 | 1.96 | 1.48 | Zfp26 |
| 93 | A_51_P221031 | 1.29 | 1.93 | 1.14 | 1.46 | 1.46 | Slc16a12 |
| 94 | A_52_P531514 | 1.64 | 1.44 | 1.29 | 1.42 | 1.45 | Uhrf2 |
| 95 | A_52_P81571 | 0.81 | 2.11 | 1.10 | 1.75 | 1.44 | Rad51l1 |
| 96 | A_52_P367294 | 0.94 | 0.82 | 1.85 | 2.17 | 1.44 | Fsd1l |
| 97 | A_52_P566840 | 1.31 | 2.52 | 0.81 | 1.14 | 1.44 | Gpr110 |
| 98 | A_51_P265806 | 2.13 | 1.26 | 1.37 | 1.00 | 1.44 | Clca2 |
| 99 | A_51_P449624 | 0.71 | 1.06 | 1.65 | 2.33 | 1.44 | 6430706D22Rik |
| 100 | A_51_P268167 | 0.75 | 1.44 | 1.64 | 1.91 | 1.44 | D14Abb1e |
| 101 | A_51_P390755 | 1.34 | 2.35 | 0.75 | 1.22 | 1.42 | 9630010G10Rik |
| 102 | A_52_P229536 | 1.12 | 0.90 | 1.67 | 1.90 | 1.40 | Cd44 |
| 103 | A_52_P172704 | 1.17 | 1.58 | 1.31 | 1.52 | 1.39 | E030011O05Rik |
| 104 | A_52_P18116 | 1.19 | 0.99 | 1.69 | 1.69 | 1.39 | Ccl24 |
| 105 | A_51_P433796 | 1.81 | 1.35 | 1.29 | 1.07 | 1.38 | Agr3 |
| 106 | A_52_P175190 | 0.99 | 2.86 | 0.79 | 0.84 | 1.37 | Egfr |
| 107 | A_51_P211165 | 0.77 | 0.63 | 1.97 | 2.12 | 1.37 | Rbm26 |
| 108 | A_51_P374707 | 1.22 | 1.50 | 1.42 | 1.34 | 1.37 | Tspan12 |
| 109 | A_52_P288177 | 1.29 | 1.28 | 1.27 | 1.62 | 1.36 | Epha4 |
| 110 | A_52_P655285 | 1.37 | 1.10 | 1.46 | 1.52 | 1.36 | Zfp462 |
| 111 | A_51_P483483 | 1.12 | 1.18 | 1.68 | 1.43 | 1.35 | ENSMUST00000038450 |
| 112 | A_52_P247927 | 0.98 | 1.78 | 1.42 | 1.22 | 1.35 | 2810442I21Rik |
| 113 | A_51_P474902 | 0.71 | 2.01 | 1.18 | 1.49 | 1.35 | Acvr1c |
| 114 | A_51_P426919 | 0.87 | 0.89 | 1.89 | 1.71 | 1.34 | Gt(ROSA)26Sor |
| 115 | A_52_P507479 | 1.84 | 1.39 | 0.82 | 1.25 | 1.33 | Fam73a |
| 116 | A_51_P335000 | 1.82 | 0.92 | 1.49 | 0.99 | 1.30 | Fhl1 |
| 117 | A_52_P308875 | 1.41 | 1.90 | 0.93 | 0.98 | 1.30 | Apobec3 |
| 118 | A_51_P430071 | 0.95 | 1.14 | 1.61 | 1.52 | 1.30 | Hist2h2be |
| 119 | A_52_P290325 | 0.71 | 1.25 | 1.58 | 1.67 | 1.30 | Depdc1a |
| 120 | A_51_P170725 | 1.08 | 1.59 | 1.30 | 1.22 | 1.30 | 1300002K09Rik |
| 121 | A_52_P187987 | 1.52 | 1.35 | 1.14 | 1.16 | 1.29 | 2310001H17Rik |
| 122 | A_52_P486260 | 1.97 | 1.57 | 0.96 | 0.66 | 1.29 | Prelp |
| 123 | A_52_P18706 | 1.53 | 1.22 | 1.01 | 1.33 | 1.27 | Dppa5a |
| 124 | A_51_P120066 | 1.54 | 0.70 | 1.52 | 1.32 | 1.27 | 9330151L19Rik |
| 125 | A_51_P517843 | 1.36 | 1.41 | 1.12 | 1.13 | 1.26 | Glipr2 |
| 126 | A_52_P60194 | 1.49 | 1.15 | 1.09 | 1.31 | 1.26 | C4bp |
| 127 | A_52_P639343 | 0.94 | 1.69 | 1.11 | 1.28 | 1.26 | D330028D13Rik |
| 128 | A_51_P474538 | 1.00 | 0.75 | 1.43 | 1.84 | 1.25 | Igf1r |
| 129 | A_52_P669035 | 0.95 | 1.51 | 1.28 | 1.27 | 1.25 | Clca1 |
| 130 | A_52_P79648 | 0.66 | 0.93 | 1.72 | 1.70 | 1.25 | Ocm |
| 131 | A_52_P25932 | 0.80 | 1.22 | 1.32 | 1.66 | 1.25 | TC1660846 |
| 132 | A_52_P363110 | 0.85 | 1.57 | 1.25 | 1.33 | 1.25 | Fgfr2 |
| 133 | A_52_P674165 | 0.95 | 0.93 | 1.89 | 1.23 | 1.25 | Rsbn1l |
| 134 | A_51_P105178 | 1.13 | 0.88 | 1.40 | 1.59 | 1.25 | Sox4 |
| 135 | A_52_P652293 | 1.59 | 1.37 | 1.25 | 0.78 | 1.25 | Trim24 |
| 136 | A_52_P377791 | 1.22 | 1.24 | 1.40 | 1.10 | 1.24 | Fxyd3 |
| 137 | A_52_P652289 | 0.62 | 0.69 | 1.68 | 1.96 | 1.24 | AK032921 |
| 138 | A_51_P492707 | 1.91 | 1.24 | 0.93 | 0.87 | 1.24 | Ptpro |
| 139 | A_52_P239536 | 1.00 | 0.97 | 1.47 | 1.50 | 1.24 | Ppp1r9a |
| 140 | A_52_P425317 | 1.44 | 0.88 | 1.31 | 1.32 | 1.24 | 4933406C10Rik |
| 141 | A_52_P67570 | 0.73 | 1.18 | 1.27 | 1.75 | 1.23 | Lass4 |
| 142 | A_52_P454703 | 0.64 | 1.41 | 1.33 | 1.54 | 1.23 | BC033430 |
| 143 | A_51_P327369 | 0.82 | 0.95 | 1.43 | 1.70 | 1.23 | Plekhg3 |
| 144 | A_52_P641684 | 0.79 | 1.15 | 1.37 | 1.60 | 1.23 | Nfia |
| 145 | A_52_P479539 | 0.81 | 0.93 | 1.71 | 1.41 | 1.22 | Cit |
| 146 | A_51_P323248 | 0.99 | 0.80 | 1.57 | 1.50 | 1.22 | Sdc4 |
| 147 | A_52_P491849 | 0.65 | 0.60 | 1.64 | 1.95 | 1.21 | Trp53 |
| 148 | A_52_P742248 | 0.64 | 0.58 | 1.77 | 1.86 | 1.21 | Als2cr13 |
| 149 | A_51_P394172 | 1.21 | 1.87 | 0.83 | 0.93 | 1.21 | Es1 |
| 150 | A_51_P168439 | 1.16 | 1.19 | 1.34 | 1.13 | 1.20 | Klhl24 |
| 151 | A_52_P661071 | 1.03 | 0.63 | 1.57 | 1.57 | 1.20 | Snhg3 |
| 152 | A_51_P409694 | 0.90 | 1.87 | 0.96 | 1.06 | 1.20 | Psrc1 |
| 153 | A_52_P481770 | 1.55 | 0.66 | 0.90 | 1.67 | 1.19 | NAP103155-1 |
| 154 | A_52_P260747 | 0.97 | 1.60 | 1.20 | 1.00 | 1.19 | E430016P22Rik |
| 155 | A_52_P130044 | 0.75 | 0.60 | 1.50 | 1.89 | 1.18 | TC1632596 |
| 156 | A_51_P244586 | 1.05 | 1.12 | 1.14 | 1.40 | 1.18 | Zfp618 |
| 157 | A_51_P110301 | 0.83 | 1.54 | 1.20 | 1.13 | 1.18 | C3 |
| 158 | A_51_P358445 | 0.87 | 1.15 | 1.34 | 1.34 | 1.17 | Dnase1 |
| 159 | A_51_P517672 | 0.72 | 1.12 | 1.41 | 1.44 | 1.17 | Rnf152 |
| 160 | A_52_P61893 | 0.91 | 0.58 | 1.47 | 1.72 | 1.17 | Nono |
| 161 | A_51_P277588 | 1.03 | 1.12 | 1.31 | 1.21 | 1.17 | Sfrs18 |
| 162 | A_51_P237924 | 1.00 | 0.83 | 1.13 | 1.69 | 1.16 | Slc6a7 |
| 163 | A_52_P1115511 | 1.76 | 1.15 | 0.96 | 0.77 | 1.16 | 6030422H21Rik |
| 164 | A_52_P10683 | 1.22 | 0.87 | 1.24 | 1.28 | 1.15 | Hist1h1d |
| 165 | A_52_P996032 | 0.85 | 0.70 | 1.33 | 1.71 | 1.15 | A630031M23Rik |
| 166 | A_51_P293729 | 0.97 | 0.65 | 1.39 | 1.58 | 1.14 | Ivns1abp |
| 167 | A_52_P485417 | 0.84 | 0.73 | 1.29 | 1.70 | 1.14 | Hmgb2 |
| 168 | A_52_P254095 | 0.78 | 0.78 | 1.71 | 1.29 | 1.14 | Cd200 |
| 169 | A_52_P481493 | 1.01 | 0.72 | 1.33 | 1.50 | 1.14 | Fkbp5 |
| 170 | A_52_P244193 | 0.86 | 1.07 | 1.36 | 1.24 | 1.14 | Cd24a |
| 171 | A_51_P489779 | 1.22 | 0.87 | 1.39 | 1.06 | 1.13 | Cdk6 |
| 172 | A_51_P352968 | 1.18 | 1.39 | 0.86 | 1.11 | 1.13 | Marcks |
| 173 | A_52_P590701 | 0.73 | 1.51 | 1.31 | 0.98 | 1.13 | Dzip3 |
| 174 | A_51_P419726 | 1.37 | 1.13 | 1.04 | 0.97 | 1.13 | Ptprs |
| 175 | A_51_P151516 | 1.32 | 1.72 | 0.74 | 0.73 | 1.13 | B230208H11Rik |
| 176 | A_52_P232433 | 1.07 | 0.60 | 1.37 | 1.47 | 1.13 | Hnrnpa1 |
| 177 | A_51_P335770 | 1.12 | 1.39 | 1.17 | 0.81 | 1.12 | Afap1 |
| 178 | A_52_P355751 | 1.19 | 1.19 | 1.11 | 0.99 | 1.12 | 544988 |
| 179 | A_52_P1148292 | 0.82 | 1.47 | 1.10 | 1.08 | 1.12 | AK168746 |
| 180 | A_52_P679152 | 0.64 | 0.90 | 1.40 | 1.51 | 1.11 | Clk1 |
| 181 | A_51_P364657 | 1.36 | 1.39 | 0.75 | 0.90 | 1.10 | Rnf32 |
| 182 | A_51_P509746 | 1.98 | 0.73 | 0.93 | 0.76 | 1.10 | Porcn |
| 183 | A_51_P127681 | 1.04 | 1.28 | 1.10 | 0.96 | 1.10 | Clic4 |
| 184 | A_51_P102607 | 1.13 | 0.99 | 0.93 | 1.33 | 1.10 | 2310007H11Rik |
| 185 | A_51_P444696 | 0.62 | 0.70 | 1.34 | 1.71 | 1.09 | Cdca2 |
| 186 | A_52_P409076 | 1.23 | 1.41 | 0.81 | 0.91 | 1.09 | Sema5a |
| 187 | A_51_P425962 | 0.77 | 1.05 | 1.44 | 1.10 | 1.09 | Khdrbs3 |
| 188 | A_52_P146848 | 0.59 | 0.69 | 1.56 | 1.51 | 1.09 | BF120285 |
| 189 | A_51_P250217 | 1.30 | 1.30 | 0.84 | 0.90 | 1.09 | A_51_P250217 |
| 190 | A_52_P93284 | 1.68 | 0.75 | 1.09 | 0.82 | 1.08 | Mycl1 |
| 191 | A_52_P666698 | 0.77 | 1.40 | 0.72 | 1.43 | 1.08 | TC1715877 |
| 192 | A_51_P181751 | 1.22 | 0.71 | 1.39 | 1.00 | 1.08 | 4930488E11Rik |
| 193 | A_51_P419319 | 1.04 | 0.84 | 1.15 | 1.28 | 1.08 | Aqp4 |
| 194 | A_51_P216605 | 0.75 | 1.20 | 1.31 | 1.04 | 1.08 | Hbp1 |
| 195 | A_52_P206002 | 1.69 | 1.21 | 0.73 | 0.67 | 1.07 | Defcr22 |
| 196 | A_52_P319161 | 0.87 | 0.59 | 1.31 | 1.48 | 1.06 | Stmn1 |
| 197 | A_52_P15073 | 0.61 | 0.92 | 1.24 | 1.46 | 1.06 | 4833442J19Rik |
| 198 | A_51_P408749 | 1.37 | 1.01 | 1.20 | 0.66 | 1.06 | 4933439C10Rik |
| 199 | A_52_P646112 | 0.76 | 0.63 | 1.47 | 1.36 | 1.06 | Rps13 |
| 200 | A_51_P472481 | 1.46 | 1.32 | 0.78 | 0.67 | 1.06 | Dync2li1 |
| 201 | A_51_P147651 | 0.73 | 0.71 | 1.10 | 1.63 | 1.04 | Ccdc15 |
| 202 | A_51_P173735 | 0.62 | 1.32 | 0.94 | 1.28 | 1.04 | Dock11 |
| 203 | A_51_P147714 | 0.62 | 1.22 | 1.05 | 1.25 | 1.03 | Sfrs14 |
| 204 | A_52_P65295 | 0.89 | 0.73 | 0.99 | 1.52 | 1.03 | AK084936 |
| 205 | A_52_P223508 | 0.73 | 1.46 | 0.99 | 0.94 | 1.03 | 6720401G13Rik |
| 206 | A_52_P162957 | 1.04 | 0.98 | 1.06 | 1.03 | 1.03 | Frat2 |
| 207 | A_51_P429197 | 0.70 | 0.66 | 0.98 | 1.76 | 1.03 | Csnk1e |
| 208 | A_52_P555537 | 1.25 | 0.95 | 1.02 | 0.87 | 1.02 | 2810008D09Rik |
| 209 | A_51_P443322 | 0.82 | 0.85 | 0.91 | 1.50 | 1.02 | Eif3c |
| 210 | A_52_P622850 | 0.85 | 0.80 | 1.42 | 0.99 | 1.02 | Hes5 |
| 211 | A_52_P557265 | 1.18 | 1.05 | 0.80 | 1.01 | 1.01 | Cyp39a1 |
| 212 | A_51_P104125 | 0.72 | 0.60 | 1.41 | 1.31 | 1.01 | Fam76b |
| 213 | A_51_P303332 | 0.83 | 0.97 | 1.19 | 1.03 | 1.00 | Mitd1 |
| 214 | A_51_P404077 | 1.38 | 1.13 | 0.71 | 0.78 | 1.00 | Fzd2 |
| 215 | A_51_P225781 | 1.30 | 0.97 | 0.83 | 0.90 | 1.00 | Insc |
| 216 | A_52_P251366 | 1.10 | 0.97 | 0.95 | 0.95 | 0.99 | Neil3 |
| 217 | A_52_P598309 | 0.73 | 0.81 | 1.21 | 1.22 | 0.99 | 1500012F01Rik |
| 218 | A_52_P124083 | 0.72 | 1.13 | 1.09 | 1.01 | 0.99 | A530082C11Rik |
| 219 | A_52_P478289 | 0.60 | 0.81 | 1.23 | 1.29 | 0.98 | C430048L16Rik |
| 220 | A_52_P569218 | 1.06 | 0.82 | 1.08 | 0.96 | 0.98 | Utrn |
| 221 | A_51_P482043 | 0.88 | 0.72 | 1.24 | 1.05 | 0.97 | Epm2aip1 |
| 222 | A_51_P407726 | 1.10 | 1.08 | 0.84 | 0.86 | 0.97 | Zan |
| 223 | A_51_P336599 | 1.46 | 0.79 | 0.85 | 0.78 | 0.97 | Kcne3 |
| 224 | A_52_P317246 | 1.62 | 0.75 | 0.67 | 0.84 | 0.97 | Esrrg |
| 225 | A_51_P306017 | 1.59 | 0.71 | 0.72 | 0.85 | 0.97 | Dll1 |
| 226 | A_51_P199098 | 0.92 | 0.76 | 1.05 | 1.14 | 0.97 | Tmem107 |
| 227 | A_51_P488333 | 0.63 | 0.67 | 1.09 | 1.47 | 0.97 | A130022F02Rik |
| 228 | A_52_P562872 | 0.66 | 0.90 | 1.06 | 1.24 | 0.96 | 4930520O04Rik |
| 229 | A_51_P184796 | 0.75 | 1.17 | 0.85 | 1.08 | 0.96 | Zfp101 |
| 230 | A_51_P288277 | 1.12 | 0.76 | 1.14 | 0.83 | 0.96 | AK003987 |
| 231 | A_51_P251438 | 0.96 | 0.99 | 0.91 | 0.99 | 0.96 | 9130004C02Rik |
| 232 | A_51_P434332 | 1.15 | 1.23 | 0.74 | 0.71 | 0.96 | Sycn |
| 233 | A_52_P561073 | 1.16 | 0.99 | 0.82 | 0.86 | 0.96 | Tox |
| 234 | A_51_P117666 | 0.76 | 0.61 | 1.18 | 1.27 | 0.96 | 1810032O08Rik |
| 235 | A_52_P650259 | 1.16 | 0.92 | 0.88 | 0.85 | 0.95 | Noxa1 |
| 236 | A_51_P337662 | 0.80 | 0.74 | 1.23 | 1.02 | 0.95 | Ddx26b |
| 237 | A_51_P402760 | 0.67 | 1.45 | 0.74 | 0.93 | 0.95 | Pla2g4a |
| 238 | A_51_P169564 | 0.79 | 0.66 | 0.86 | 1.47 | 0.94 | 5730455P16Rik |
| 239 | A_52_P139936 | 1.15 | 0.59 | 1.36 | 0.67 | 0.94 | Phf21b |
| 240 | A_51_P261428 | 1.13 | 0.95 | 0.70 | 0.98 | 0.94 | Pdcd4 |
| 241 | A_51_P399305 | 0.90 | 1.26 | 0.65 | 0.93 | 0.93 | Tnfrsf19 |
| 242 | A_52_P440729 | 1.02 | 1.27 | 0.60 | 0.83 | 0.93 | Atf7ip |
| 243 | A_51_P416846 | 1.15 | 1.02 | 0.84 | 0.72 | 0.93 | 2610020H08Rik |
| 244 | A_51_P217682 | 0.87 | 1.12 | 0.99 | 0.74 | 0.93 | Pcf11 |
| 245 | A_51_P101621 | 0.58 | 0.63 | 1.07 | 1.43 | 0.93 | Creb1 |
| 246 | A_51_P348325 | 1.04 | 0.81 | 0.65 | 1.20 | 0.93 | Wdr51b |
| 247 | A_51_P155997 | 0.65 | 0.71 | 1.31 | 1.03 | 0.92 | Bcl7a |
| 248 | A_51_P300337 | 1.18 | 0.97 | 0.76 | 0.78 | 0.92 | Csrp2 |
| 249 | A_51_P146126 | 0.78 | 0.66 | 1.49 | 0.77 | 0.92 | Cdkn1b |
| 250 | A_52_P678800 | 0.90 | 0.81 | 1.01 | 0.97 | 0.92 | Rps21 |
| 251 | A_52_P329054 | 0.80 | 0.69 | 1.24 | 0.96 | 0.92 | Cep70 |
| 252 | A_52_P234039 | 0.85 | 0.84 | 1.18 | 0.80 | 0.92 | Znrf3 |
| 253 | A_52_P558368 | 0.98 | 0.90 | 1.05 | 0.74 | 0.92 | Zdhhc1 |
| 254 | A_52_P337246 | 0.81 | 1.39 | 0.71 | 0.75 | 0.92 | Isl1 |
| 255 | A_52_P301035 | 1.20 | 1.11 | 0.73 | 0.62 | 0.91 | Rccd1 |
| 256 | A_52_P90257 | 0.72 | 0.81 | 1.02 | 1.10 | 0.91 | TC1668402 |
| 257 | A_52_P148212 | 0.73 | 1.26 | 0.88 | 0.77 | 0.91 | C79407 |
| 258 | A_51_P366931 | 0.67 | 0.77 | 1.11 | 1.10 | 0.91 | Prc1 |
| 259 | A_52_P3825 | 0.96 | 1.07 | 0.73 | 0.90 | 0.91 | Ddb2 |
| 260 | A_52_P206762 | 0.73 | 0.86 | 1.04 | 1.01 | 0.91 | Zfp422 |
| 261 | A_51_P272283 | 0.59 | 1.02 | 0.98 | 1.06 | 0.91 | Cmbl |
| 262 | A_51_P245796 | 1.18 | 1.10 | 0.61 | 0.74 | 0.91 | Ddit4 |
| 263 | A_51_P127215 | 0.95 | 0.84 | 1.04 | 0.80 | 0.91 | Sesn3 |
| 264 | A_52_P748067 | 1.28 | 0.78 | 0.85 | 0.71 | 0.91 | AI608034 |
| 265 | A_51_P306608 | 0.93 | 0.62 | 1.08 | 0.96 | 0.90 | Taf1d |
| 266 | A_51_P262230 | 0.63 | 0.72 | 1.39 | 0.82 | 0.89 | a2ld1 |
| 267 | A_52_P84037 | 0.59 | 0.76 | 0.93 | 1.23 | 0.88 | Socs2 |
| 268 | A_51_P157462 | 0.68 | 0.68 | 0.90 | 1.25 | 0.88 | Rgn |
| 269 | A_51_P345248 | 0.68 | 0.63 | 0.59 | 1.59 | 0.87 | Sfxn2 |
| 270 | A_52_P416756 | 0.96 | 0.70 | 0.74 | 1.09 | 0.87 | D17H6S56E-5 |
| 271 | A_51_P142896 | 0.61 | 0.94 | 0.70 | 1.24 | 0.87 | Cd59a |
| 272 | A_52_P495104 | 0.64 | 0.76 | 1.05 | 1.03 | 0.87 | Heca |
| 273 | A_51_P439970 | 0.91 | 0.62 | 0.89 | 1.01 | 0.86 | Ggh |
| 274 | A_52_P362997 | 0.90 | 1.12 | 0.58 | 0.82 | 0.86 | Cenpl |
| 275 | A_52_P395929 | 0.59 | 0.91 | 0.97 | 0.95 | 0.86 | TC1677080 |
| 276 | A_51_P475785 | 1.06 | 0.63 | 0.98 | 0.75 | 0.85 | BC052040 |
| 277 | A_52_P613498 | 0.77 | 0.79 | 0.93 | 0.92 | 0.85 | 4833420G17Rik |
| 278 | A_51_P312482 | 0.83 | 0.78 | 1.09 | 0.69 | 0.85 | Ccdc34 |
| 279 | A_51_P355906 | 1.08 | 0.79 | 0.88 | 0.64 | 0.85 | Kit |
| 280 | A_52_P556804 | 0.59 | 0.79 | 1.06 | 0.93 | 0.84 | TC1653151 |
| 281 | A_51_P334930 | 1.32 | 0.66 | 0.77 | 0.62 | 0.84 | 1810030J14Rik |
| 282 | A_51_P333923 | 0.88 | 0.93 | 0.87 | 0.67 | 0.84 | Tspan1 |
| 283 | A_52_P177161 | 0.63 | 1.02 | 0.63 | 1.06 | 0.83 | Pbrm1 |
| 284 | A_51_P126476 | 0.68 | 0.92 | 0.87 | 0.84 | 0.83 | Myo1b |
| 285 | A_52_P316933 | 0.68 | 0.62 | 0.88 | 1.10 | 0.82 | Sh3bgrl2 |
| 286 | A_52_P472936 | 0.61 | 1.32 | 0.61 | 0.74 | 0.82 | Ythdc1 |
| 287 | A_52_P136966 | 0.68 | 0.75 | 0.88 | 0.98 | 0.82 | Ift74 |
| 288 | A_52_P199776 | 1.05 | 0.96 | 0.64 | 0.59 | 0.81 | Snhg7 |
| 289 | A_52_P126266 | 0.60 | 1.27 | 0.64 | 0.74 | 0.81 | Prkab2 |
| 290 | A_51_P124784 | 0.90 | 0.75 | 0.69 | 0.90 | 0.81 | Acpl2 |
| 291 | A_52_P329256 | 1.08 | 0.58 | 0.81 | 0.74 | 0.80 | Fxn |
| 292 | A_52_P347847 | 0.65 | 1.33 | 0.60 | 0.62 | 0.80 | AU022252 |
| 293 | A_51_P221428 | 1.21 | 0.66 | 0.60 | 0.73 | 0.80 | Cbx6 |
| 294 | A_51_P129360 | 0.86 | 0.85 | 0.81 | 0.64 | 0.79 | Pthlh |
| 295 | A_51_P500713 | 0.60 | 0.64 | 0.90 | 0.98 | 0.78 | Dck |
| 296 | A_51_P114591 | 0.74 | 0.61 | 0.87 | 0.91 | 0.78 | 1110005A03Rik |
| 297 | A_51_P141502 | 0.85 | 0.78 | 0.73 | 0.70 | 0.77 | Nudt21 |
| 298 | A_51_P362959 | 0.72 | 0.73 | 0.84 | 0.71 | 0.75 | Fbxo36 |
| 299 | A_52_P684798 | 0.73 | 1.05 | 0.60 | 0.61 | 0.75 | Alms1 |
| 300 | A_51_P102096 | 0.66 | 0.75 | 0.64 | 0.93 | 0.75 | Myc |
| 301 | A_51_P268673 | 0.61 | 0.64 | 0.74 | 0.98 | 0.74 | 2210016H18Rik |
| 302 | A_51_P243623 | 0.61 | 0.85 | 0.67 | 0.66 | 0.70 | Brms1l |
| 303 | A_51_P152211 | 0.63 | 0.78 | 0.60 | 0.71 | 0.68 | Zfp386 |
| 304 | A_51_P386304 | 0.62 | 0.66 | 0.75 | 0.66 | 0.67 | Ccnl2 |
| 305 | A_51_P200819 | 0.59 | 0.70 | 0.63 | 0.61 | 0.63 | Prdx4 |
| 306 | A_51_P338961 | 0.65 | 0.58 | 0.61 | 0.60 | 0.61 | Qser1 |

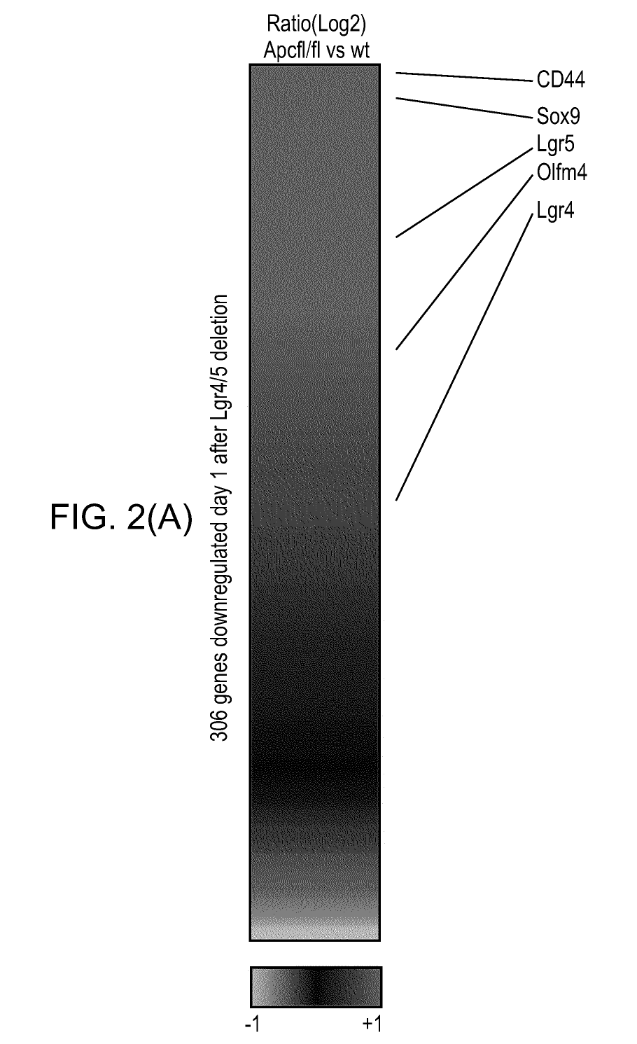

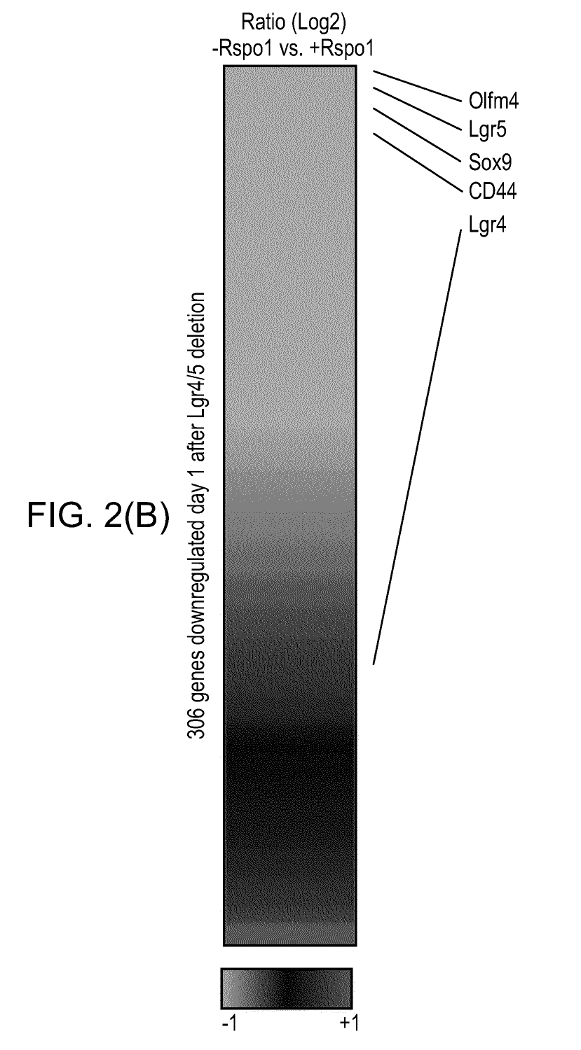

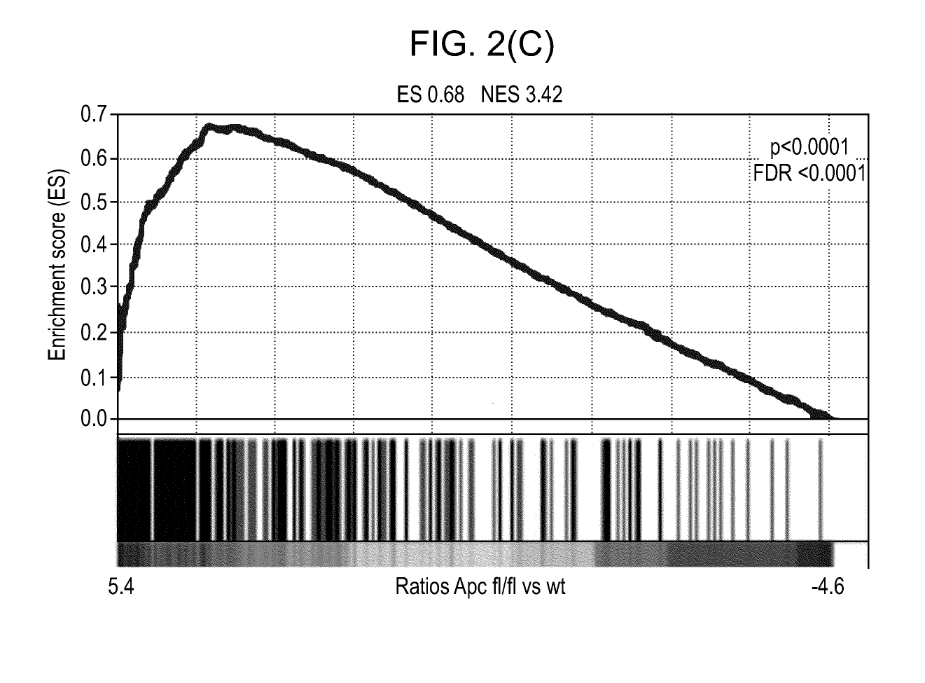

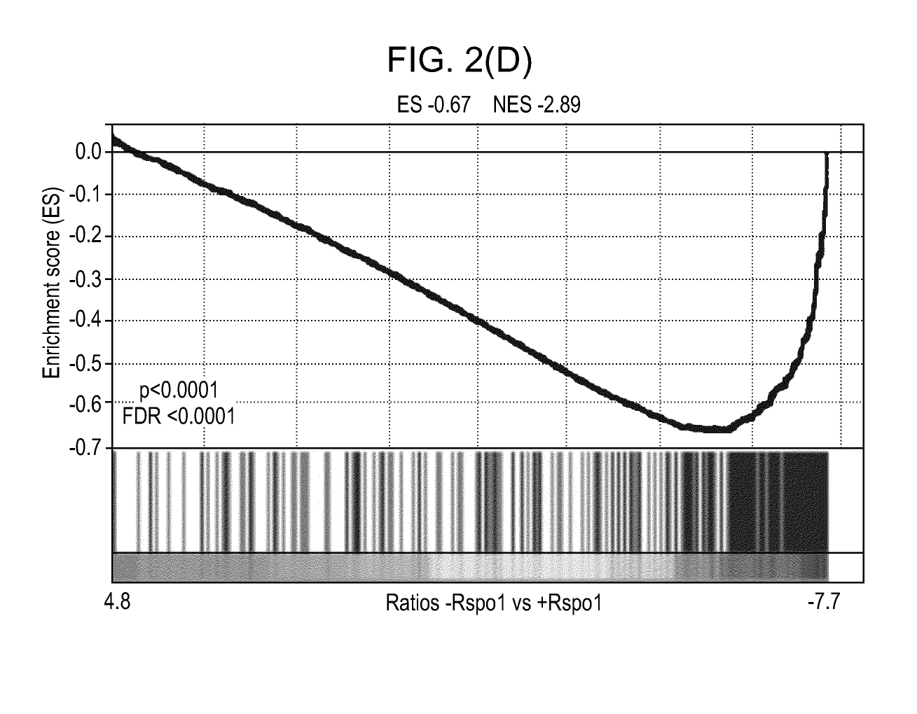

In order to determine if Lgr4/5 genes are downstream targets of the Wnt pathway, the behaviour of this gene set was analysed in two complementary scenarios that detect intestinal Wnt target genes. In the first, published scenario¹¹⁵, deletion of Apc results in the immediate upregulation of Wnt pathway target genes. Comparison of the Lgr4/5 deletion gene set to the microarray data from Sansom, O. J. et al.,¹¹⁵ showed a significant upregulation of 53% (137 genes) of the Lgr4/5 gene set, whereas only 5% (12 genes) were significantly downregulated. The remaining 42% also showed a clear tendency to be upregulated (FIG. 2, dark ratios). Next, Enrichment Analysis (GSEA) was used to determine whether the gene set is significantly enriched in a certain biological state, such as upon Wnt activation (Subramanian et al, PNAS, 2005). GSEA showed a highly significant enrichment (FDR and p-value<0.0001) of the Lgr4/5 gene set towards the upregulated genes after Apc deletion (FIG. 2C). For the second scenario, an in vitro crypt organoid culture, which is strictly dependent on the Wnt agonist Rspondin1 (Rspo1)¹¹⁶, was employed. Rspo-1 was acutely withheld from established small intestinal crypt organoids and performed differential gene expression arraying before, and 1 day after withdrawal (to be deposited at GEO). Rspo-1 withdrawal resulted in the immediate significant downregulation of 38% (166 genes) of the Lgr4/5 gene set, while only 4% (11 genes) showed the opposite behaviour. The remaining 58% showed a clear tendency towards downregulation (FIG. 2, light ratios). GSEA analysis confirmed the highly significant enrichment (FDR and p-value<0.0001) of the Lgr4/5 gene set towards the downregulated genes after Rspo-1 withdrawal (FIG. 2D). Thus, simultaneous deletion of Lgr4 and -5 resulted in the immediate downregulation of many genes that are under the control of the Wnt pathway. This suggests a previously unknown role for Lgr proteins in the Wnt signaling pathway.

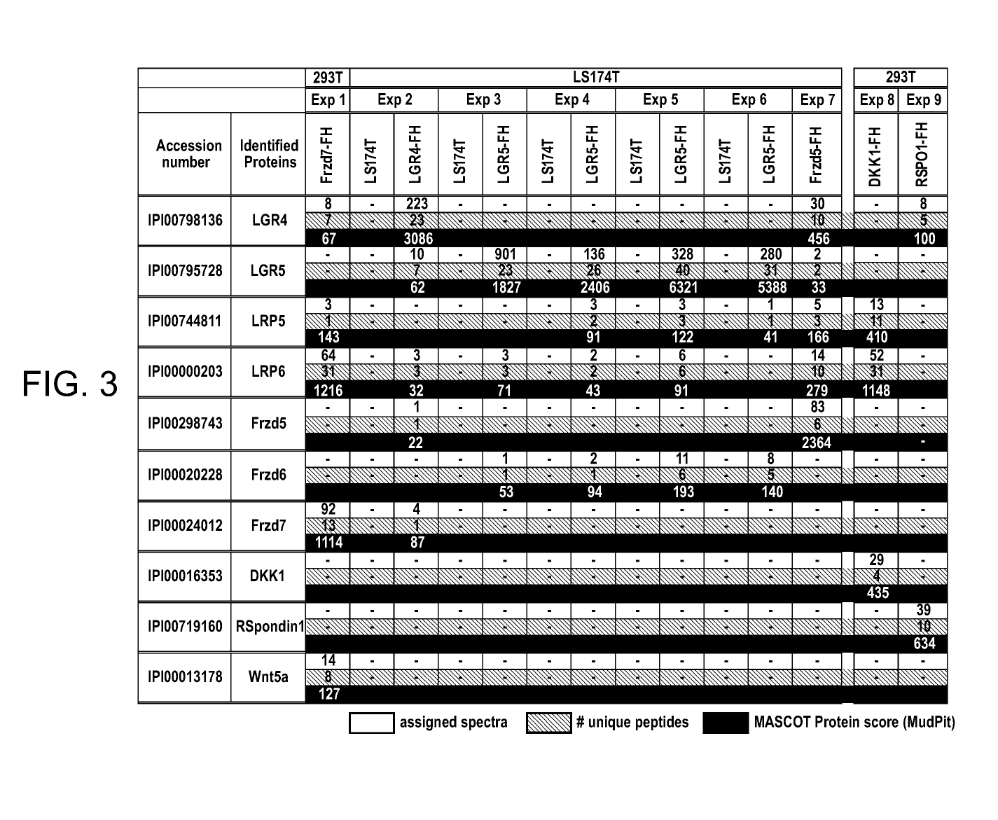

To address the molecular context in which the Lgr receptors function, a tandem affinity purification mass spectrometric strategy was pursued. Bait proteins were generated by double (Flag-HA)-tagging¹¹⁷. As a pilot, double-tagged Frizzled7 (Frz7-FH) were transiently transfected into HEK293T cells. The cells were lysed and immunoprecipitated with an anti-Flag antibody. The immunoprecipitated material was eluted under native conditions with Flag peptide, and re-precipitated with the anti-HA antibody after which mass spectrometric analysis was performed. Significant signatures were detected for Frz7 and, as expected, the Wnt co-receptors LRP5 and LRP6. However, surprisingly, multiple peptides were also detected with high confidence, belonging to endogenous Lgr4 (FIG. 3). Of note, Lgr4 or its family members were not observed using the same protocol in HEK293 cells for approximately 20 unrelated baits. Nor were Frizzleds or LRP5/6 ever observed with these bait proteins.

Stable clones of LS174T colorectal cancer cells were then generated that moderately overexpressed double-tagged versions of Lgr4, Lgr5 or Frizzled5 (Frz5). The Lgr4 bait captured Lgr5 LRP6, Frz5 and Frz7. In four independent experiments conducted with Lgr5 as bait, Frz6 and LRP5 and LRP6 were detected. Using Frz5 as bait, we identified Lgr4, Lgr5, LRP5 and LRP6. These proteins were never observed in non-transfected controls that were run in parallel. These results are summarized in FIG. 3. From this set of experiments, it was concluded that Lgr4 and Lgr5 can occur in a physical complex with Frizzleds and LRP5/6.

In an independent set of experiments, the inventors pursued the identification of the Rspo-1 receptor. The four secreted Rspondin proteins, only encoded in vertebrate genomes, activate the canonical Wnt pathway as measured by increases in β-catenin levels and in β-catenin/Tcf reporter assays. They are particularly potent when synergizing with secreted Wnt proteins^{118, 119}. Systemic delivery of Rspo-1 in mice leads to a dramatic enhancement of the size and proliferative activity of Wnt-responsive intestinal crypts¹²⁰ and stimulates repair after epithelial injury¹²¹. While Rspondin3 has recently been shown to utilize syndecans as receptors to mediate non-canonical Wnt signals¹²², the nature of Rspondin receptors that drive canonical Wnt signals has remained controversial. Rspo-1 has been reported to be a high affinity ligand of the Wnt co-receptor LRP6^{123, 124}. Rspo-1 has also been postulated to bind and block the Kremen protein that down-regulates surface expression of Wnt receptors¹²⁵. In a fourth study, Rspo-1 has been proposed to activate the Wnt pathway by blocking the interaction of the Wnt inhibitor Dkk1 with the LRP6 co-receptor¹²⁶.

HEK293T cells are responsive to Wnt3A and Rspondin¹²⁷. Therefore, the inventors decided to use this human kidney cell line in an effort to identify the cognate Rspondin receptor. In order to validate the efficacy of using soluble baits for the discovery of surface receptors, a double (Flag-HA)-tagged version of human Dkk1 (Dkk1-FH) was readily produced at ˜1 μg/ml by transient transfection into HEK293T cells. Dkk1 is known to interact with Lrp5/6 and block Wnt signaling¹²⁶. The inventors incubated 2×10⁹ HEK293T cells with the Dkk1-FH conditioned medium on ice. The cells were then washed several times, lysed and immunoprecipitated with an anti-FLAG antibody. The immunoprecipitated material was eluted under native conditions with FLAG peptide, and re-precipitated with the anti-HA antibody, after which mass spectrometric analysis was performed. In this analysis, Dkk1 and endogenous LRP5 and LRP6 (FIG. 3) were identified, proving that the strategy worked to identify surface receptors.

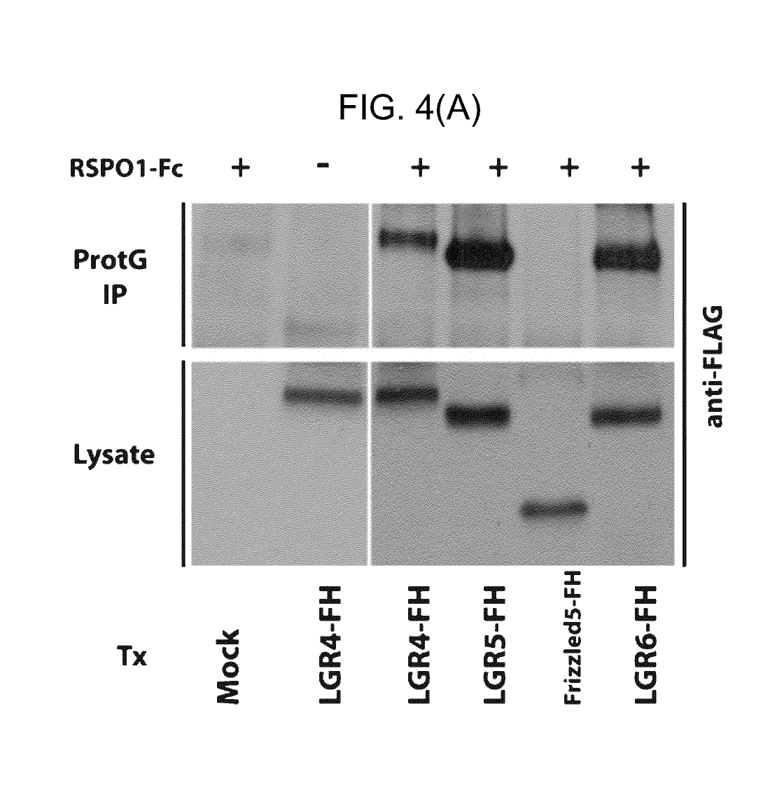

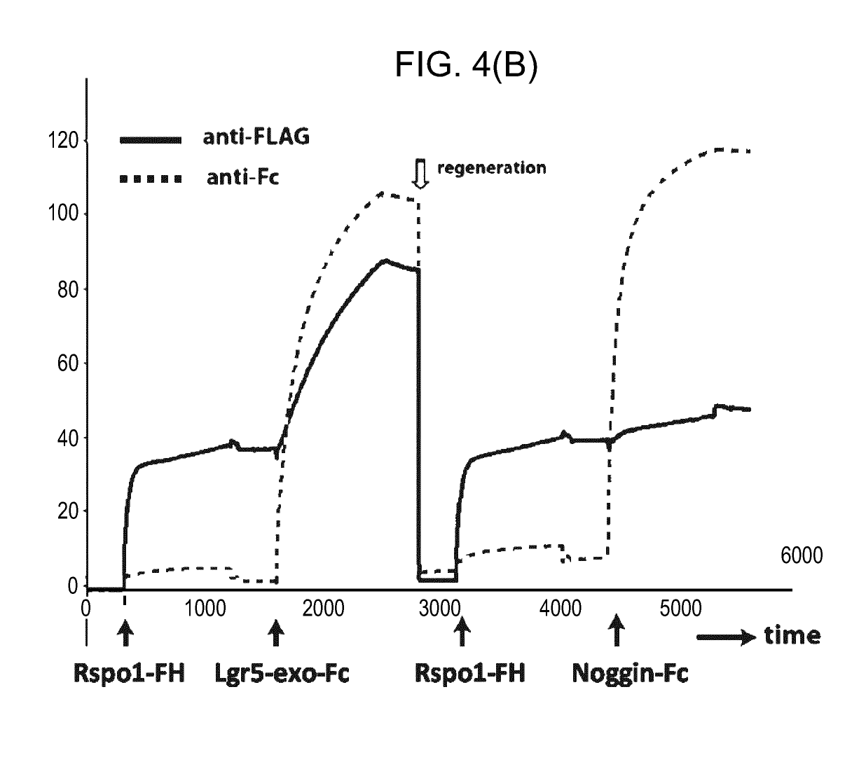

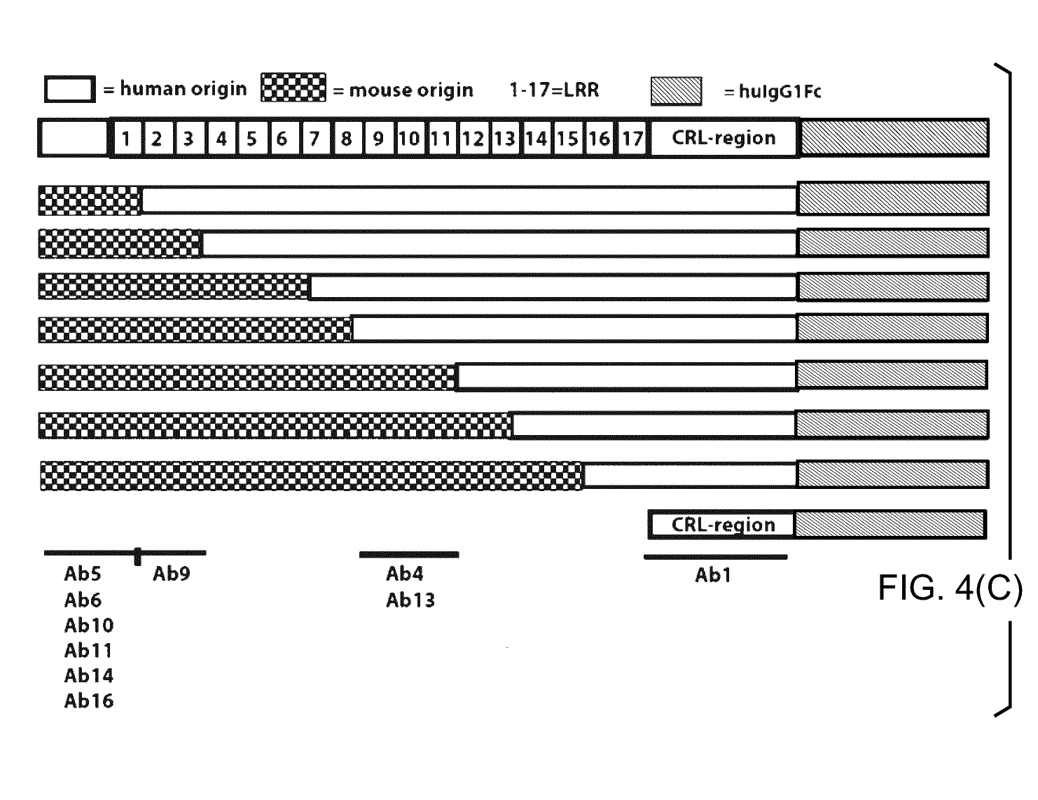

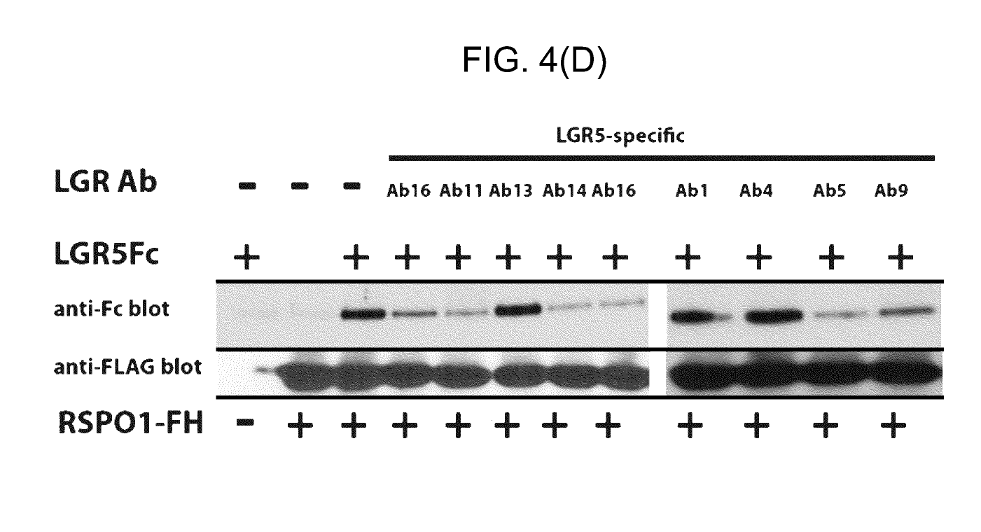

A double (FLAG-HA)-tagged version of human Rspo-1 (Rspo1-FH) was then produced at ˜1 μg/ml in HEK293T cells, the concentration that works optimally in crypt organoid culture¹¹⁶. As tested in the β-catenin/TCF reporter TOPFlash reporter assay128, the protein was functional, potentiating the effect of Wnt3A by ˜12-fold (FIG. 4). Then 2×10⁹ HEK293 cells were incubated with the Rspo1-FH conditioned medium on ice and subjected them to the above protocol. A dominant protein in terms of peptide identifications in the mass spectrometric analysis was Rspo-1. The only transmembrane protein detected was Lgr4 (FIG. 3).

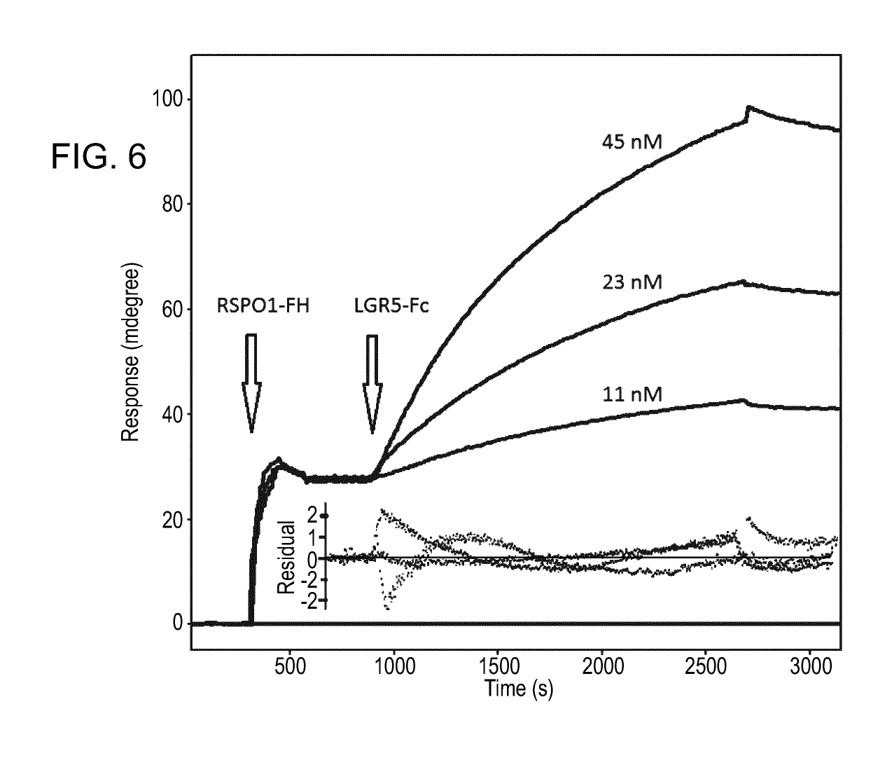

To confirm this interaction, HEK293T cells were transfected transiently with FLAG/HA-tagged versions of Lgr4, Lgr5 or Lgr6, and with tagged Frz5 as control. Transfected cells were incubated with a conditioned medium containing an Rspo1-Fc fusion protein at ˜1 μg/ml, washed, lysed and Rspo1-Fc was immunoprecipitated with proteinG beads. Western blotting for the FLAG tag revealed binding of soluble Rspo1-Fc to Lgr4, Lgr5 and Lgr6, but not to Frz5 (FIG. 4A). Soluble Rspo1-FH also interacted with the leucine-rich-repeat exodomain of Lgr5 (amino acids 1 to 546) expressed as a human IgG-Fc fusion protein (Lgr5-exo-Fc). This allowed us to perform a binding experiment using Surface Plasmon Resonance array imaging. Anti-FLAG antibody spotted on the sensor chip, readily captured Rspo-1-FH (FIG. 4B Left). After a wash, Lgr5-exo-Fc bound to Rspo1-FH (left). After regeneration, a control experiment was performed using a Noggin-Fc fusion protein (FIG. 4B, right), which did not bind Rspo1-FH. Both Lgr5-exo-Fc and Noggin-Fc could be captured on anti-human IgG spotted as a control (FIG. 4B). The K_{D} of the Lgr5-exo-Fc/Rspo1-FH interaction was determined at ˜3 nM by a 1:1 discrete interaction model at low ligand and low analyte concentration (Scrubber 2.0, BioLogic Software, Australia) (see FIG. 6).

TABLE 3
List of Mass Spectrometry baits that did not yield peptides
from Frizzled proteins, Lgr4-6, or Lrp5/6 proteins

betaTrCP1/FBXW1
betaTrCP2/FBXW11
eEF2K
ASB11
KCTD11
KCTD21
RNF43
BHLHE40
UCHL1
USP47
FBXW9
FBXW4
FBXW5
FBXO3
FBXO18
FBXO31
FBXL2
EMI1
E2F3A
E2F3B
PER2

A series of rat monoclonal antibodies was raised against the full-length human Lgr5 protein and described in WO2010/016766¹²⁹, which is incorporated herein in its entirety. Table 4 provides an overview of working names for each mAb and the corresponding original sub-clone identities.

TABLE 4
Working names and subclone IDs for anti-Lgr5 antibodies.

| Lgr5 mAb | |
|---|---|
| name | Subclone ID |
| Ab1 | 1D9 A5 |
| Ab4 | 4D11 F8 |
| Ab5 | 6C10 D4 |
| Ab6 | 3A4 G6 |
| Ab7 | 5A7 F10 |
| Ab9 | 9G5 E5 |
| Ab10 | 2B8 A7 |
| Ab11 | 3B9 D9 |
| Ab12 | 5C8 G9 |
| Ab13 | 7B11 G11 |
| Ab14 | 8F2 B4 |
| Ab16 | 10C1 C7 |

All antibodies reacted with Lgr5-exo-Fc and their epitopes were mapped using C-terminal deletion clones as well as human-mouse hybrids of the Lgr5 exodomain. The results are summarized in FIG. 4C. Preincubation of Lgr5-exo-Fc with these antibodies prior to exposure to Rspo1-FH revealed that all antibodies recognizing the region comprising the N-terminus and leucine rich repeat 1 (LRR1) of Lgr5 blocked the Rspo1 interaction, whereas the other antibodies did not (FIG. 4D).

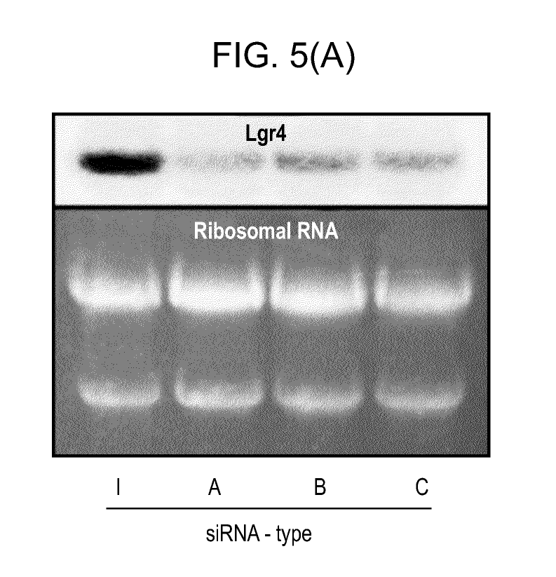

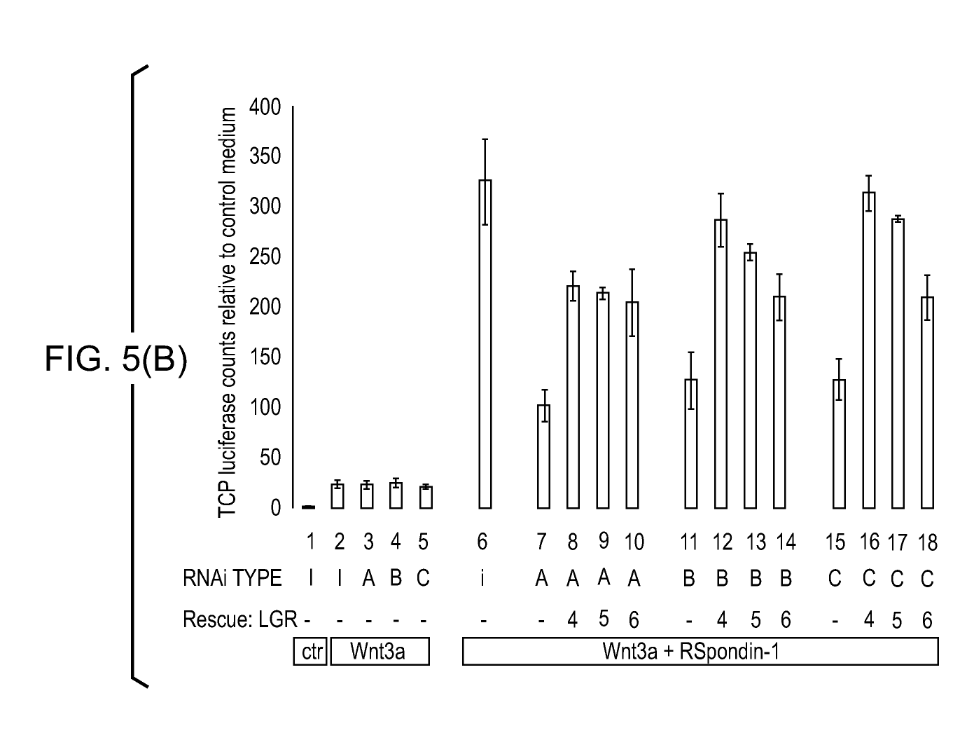

To test if Lgr4 constitutes a functional Rspo-1 receptor, Lgr4 mRNA was removed from HEK293T cells by three independent siRNAs (each of which significantly reduced Lgr4 expression as assessed by Northern blotting; FIG. 5A). Subsequently, the cells were transfected with TOPFlash Wnt reporters and their response to exogenously added Wnt3A and Rspo-1 was measured. Wnt3A alone induced a ˜25 fold increase in TOPFlash activity. Removal of Lgr4 had no effect on this response to exogenous Wnt3A (FIG. 5B; compare bar 1 to bar 2-5). The combination of Wnt3A with Rspo-1 led to a further ˜12 fold increase in TOPFlash activity (bar 6). This effect was greatly diminished in cells from which Lgr4 was removed (FIG. 5B; bars 7, 10 and 14), but could be rescued by transfection of very low amounts (5 ng) of Lgr4 (bars 8, 12 and 16), Lgr5 (bars 9, 13 and 17) or Lgr6 expression plasmids (bars 10, 14 and 18). This implied that Lgr4 is dispensable for Wnt3A-mediated input into this pathway, but is necessary for transmitting Rspo-1-mediated input into the canonical Wnt pathway. Thus, Lgr4 is a facultative component of the Wnt receptor complex. Given that Lgr5 and Lgr6 also bind Rspo-1 and can functionally rescue loss of Lgr4, the three family members appear to fulfill similar biochemical roles.

Taken together, these data imply that the members of the Lgr4/5/6 family reside in Frizzled-LRPS/6 complexes and bind soluble Rspondins. Engagement of Lgr proteins by Rspondins triggers downstream canonical Wnt signals through the associated Frizzled-Lrp5/6 complex. This notion is in agreement with the observation that Rspondins, like Wnt proteins, induce LRP6 phosphorylation¹²⁴. The current data do not immediately explain why Rspondin signalling appears dependent on the presence of Wnt proteins. Possibly, Wnt interactions with their cognate Frizzled-Lrp5/6 complexes induce conformational or biochemical changes in the receptor complex that are essential for the subsequent enhancement of signalling activity by Rspondin/Lgr interaction.

The inventors have previously shown that Lgr5 is itself a Wnt target gene and marks stem cells in multiple Wnt-dependent adult stem cell compartments. The current observations provide a molecular explanation for the expression of Lgr5 and the related gene Lgr4 by Wnt-dependent stem- and progenitor cells. In intestinal crypts, Lgr4 and Lgr5, incorporated into Frizzled/LRP complexes, allow Rspondins to augment short-range Wnt signals emanating from Paneth cells¹²⁸. Crypt stem cells co-express Lgr4 and Lgr5, while their undifferentiated progenitors only express Lgr4. Indeed, we show here that the combined deletion of Lgr4 and -5 phenocopies loss of Wnt signaling activity. Lgr4 and Lgr5 may only differ in their expression pattern, or the signals transmitted through Lgr4 and Lgr5 proteins may be subtly different, such that Lgr5 would provide a stem cell-specific version of Wnt signal potentiation by Rspondins.

Overall, these surprising results demonstrate a previously unknown mechanism for Rsponding signalling through Lgr protein receptors. Unexpectedly, Lgr proteins form part of a Wnt receptor complex (Lgr-Frz-LRP) for activating Wnt/β-signalling. Now that it is known that this receptor complex has two ligands—Wnt and Rspondin—this opens up new opportunities to design Rspondin mimetics, which mimic the role of Rspondin in activating Wnt/β-signalling. Such mimetic compounds could be used alone, or in combination with conventional Wnt agonists, for therapeutic reasons, as well as for tissue engineering and cell culture.

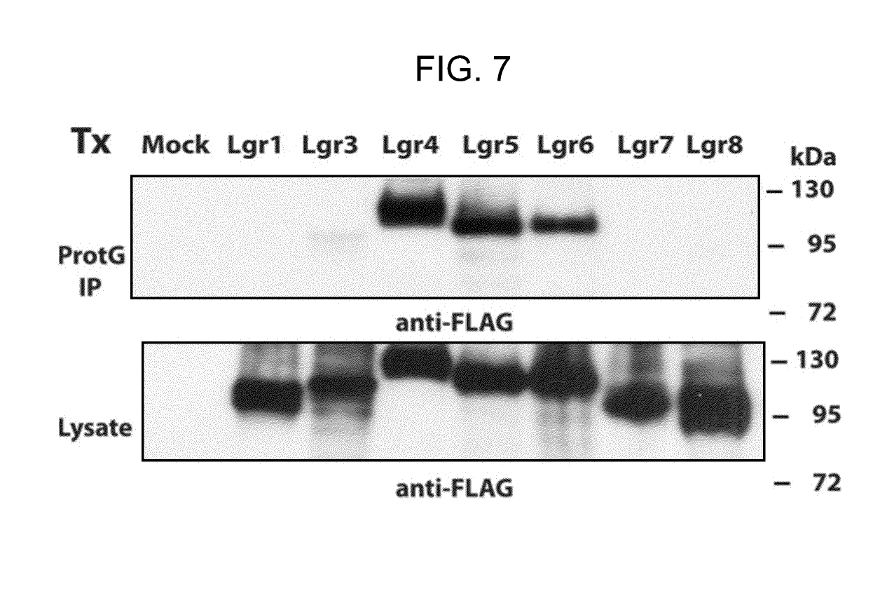

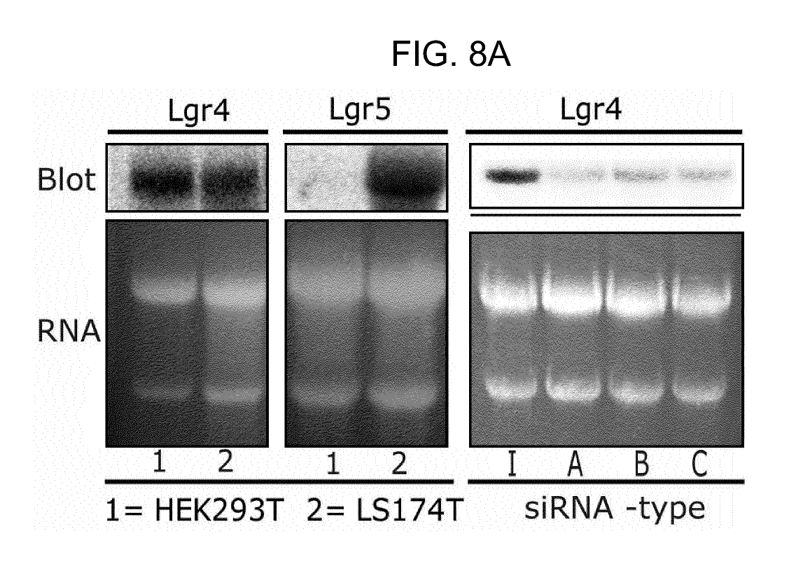

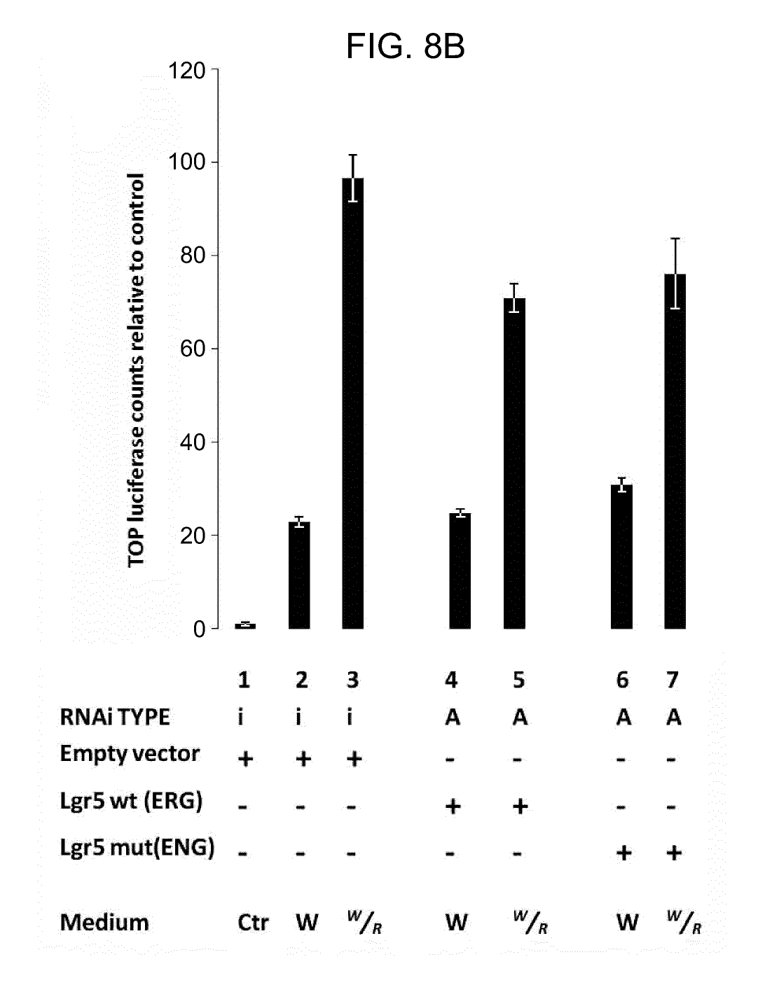

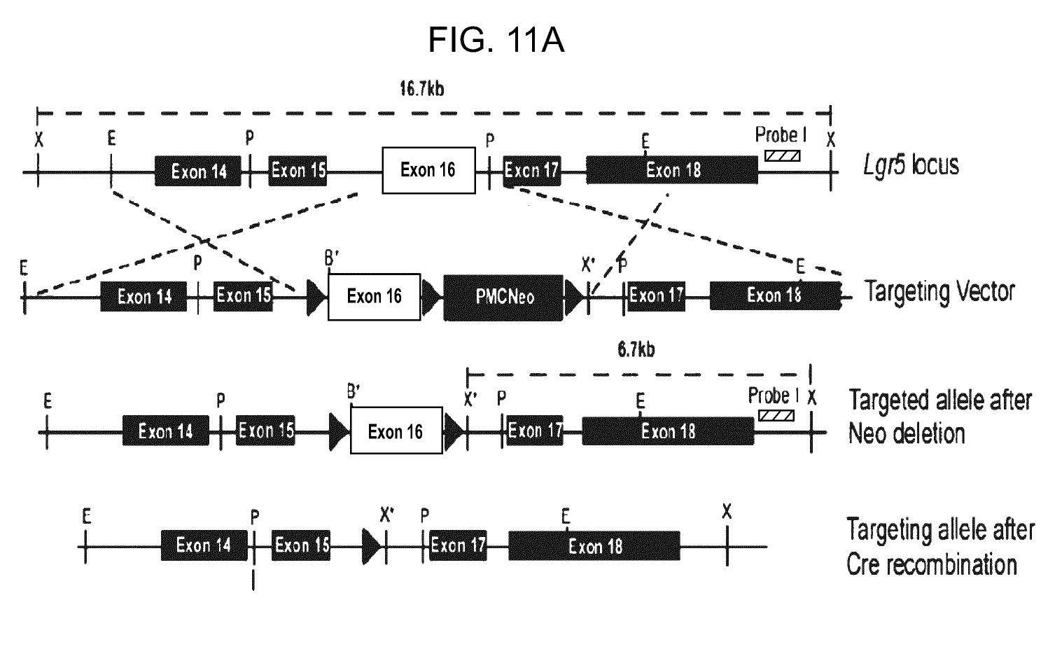

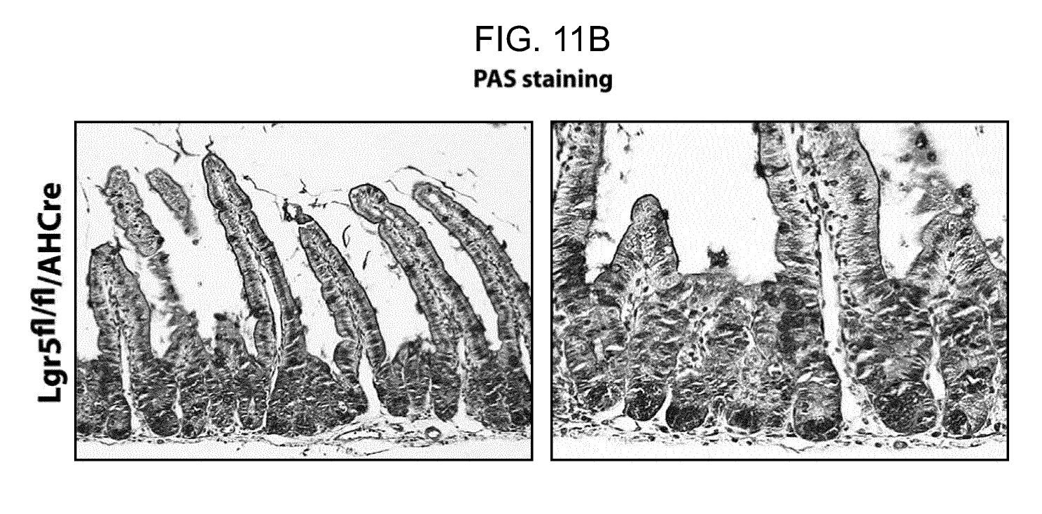

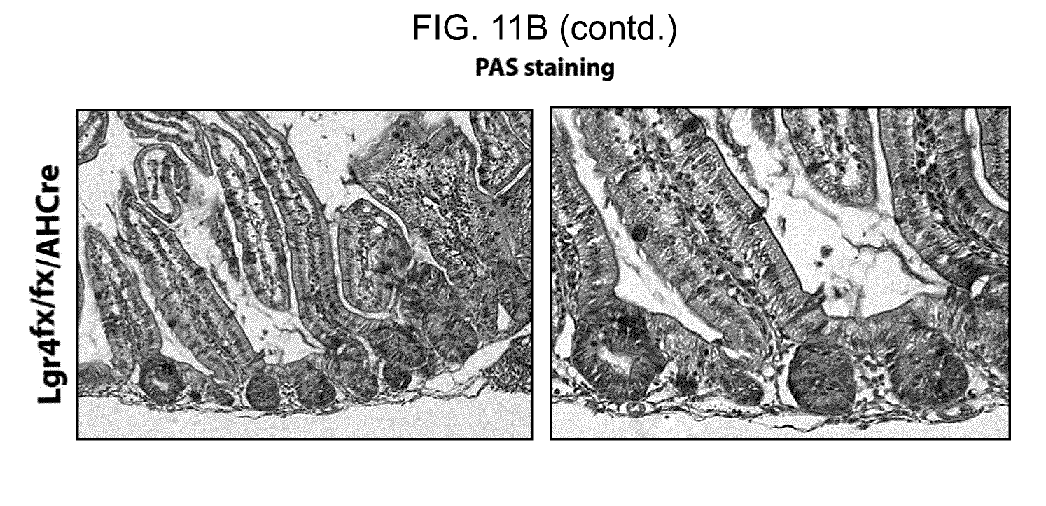

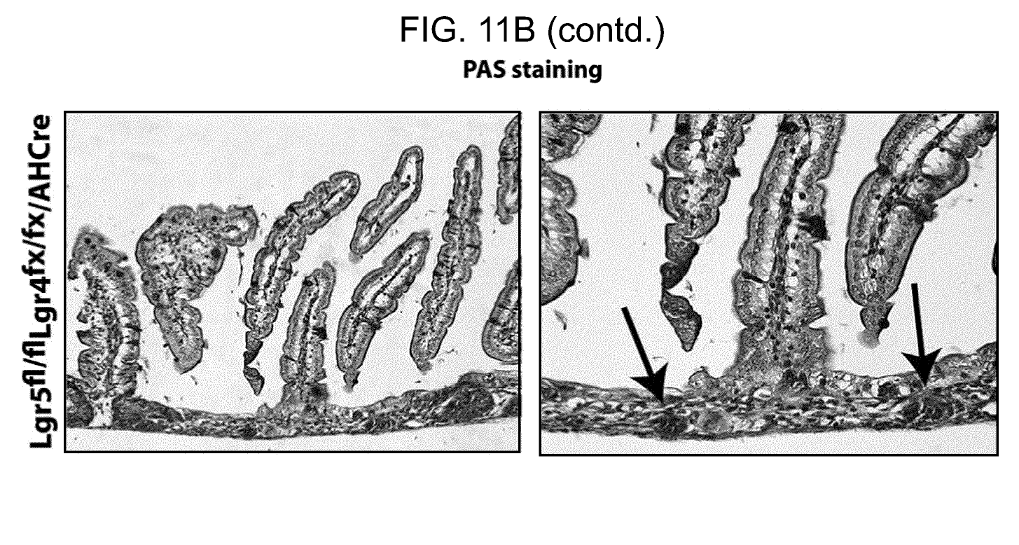

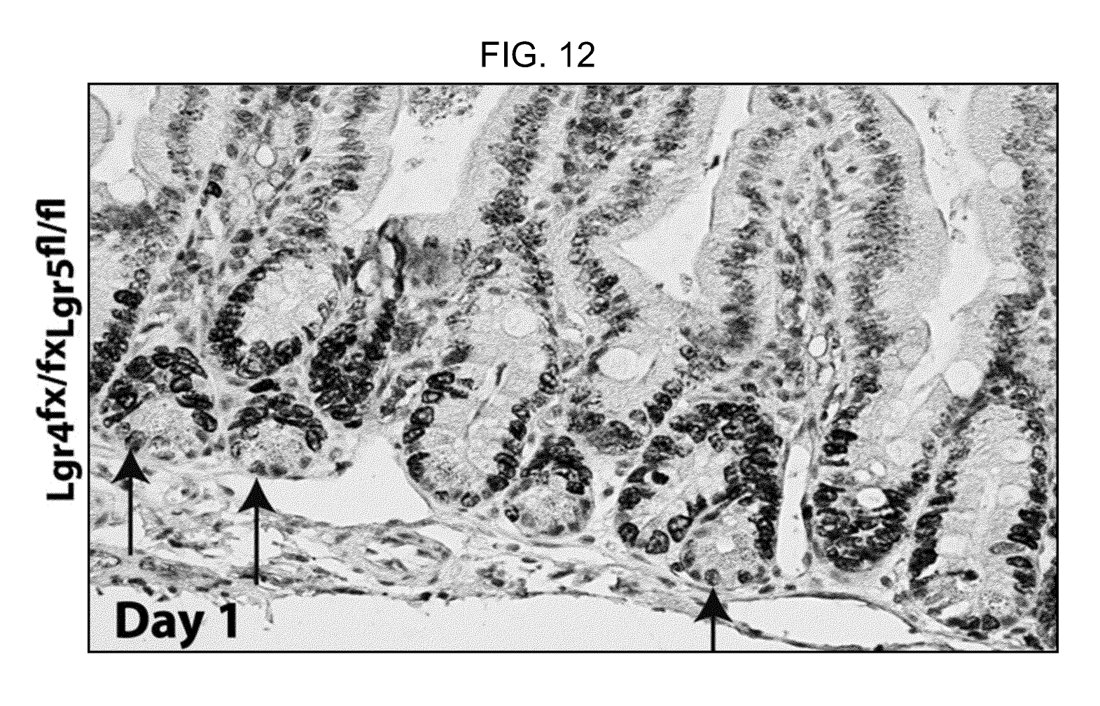

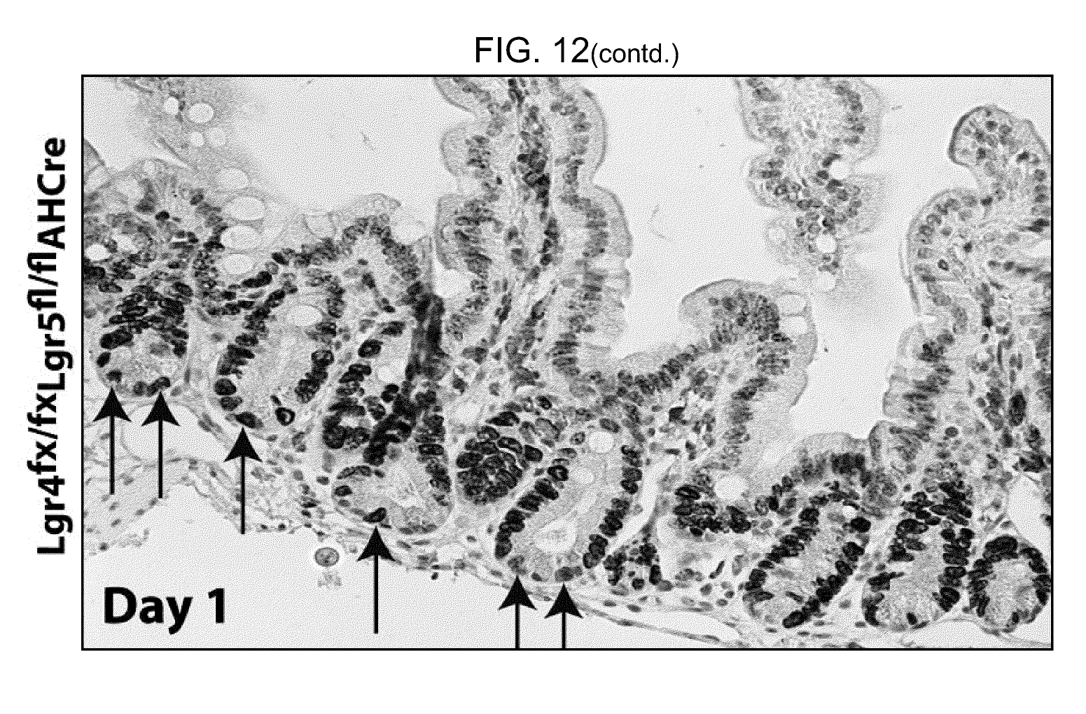

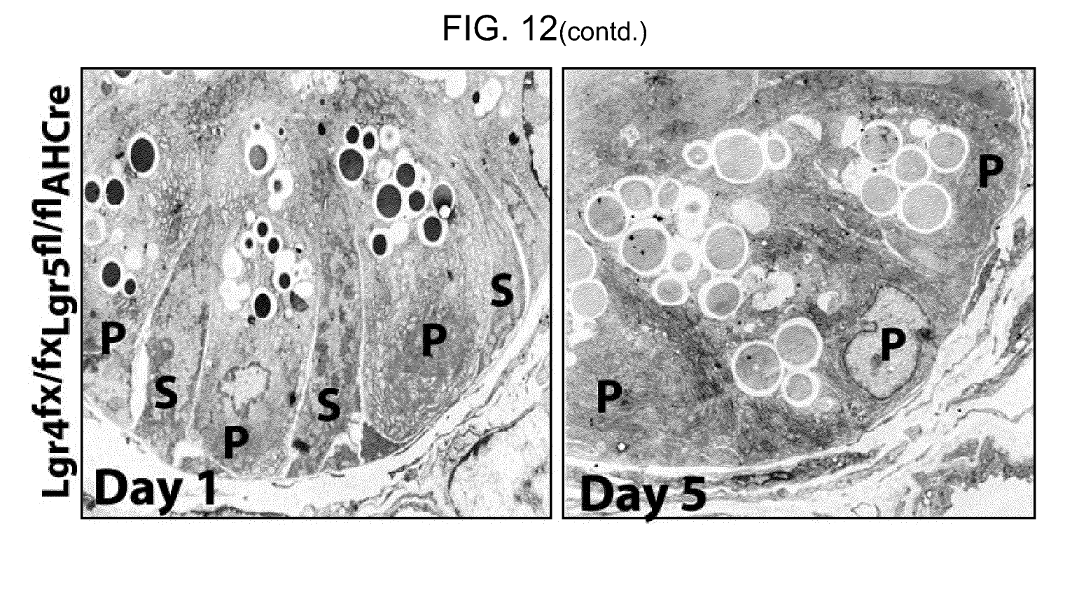

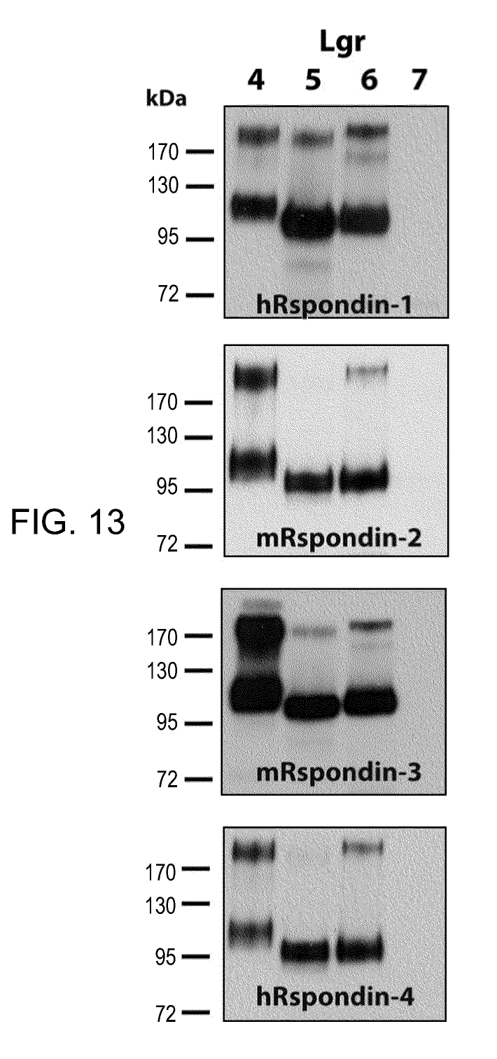

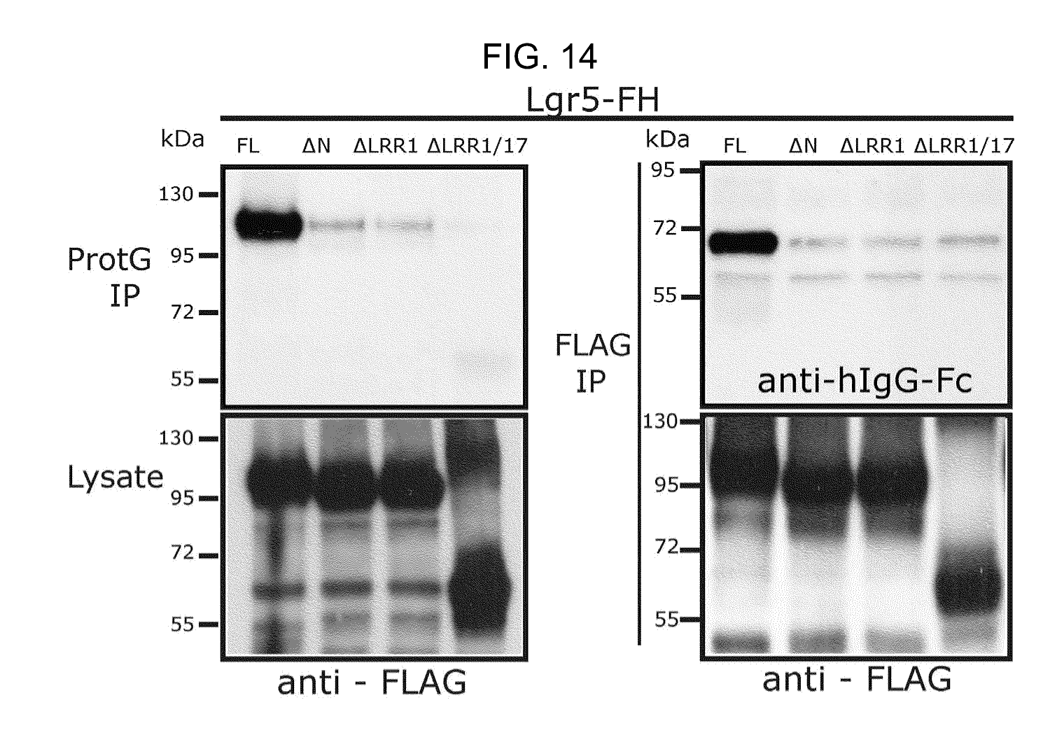

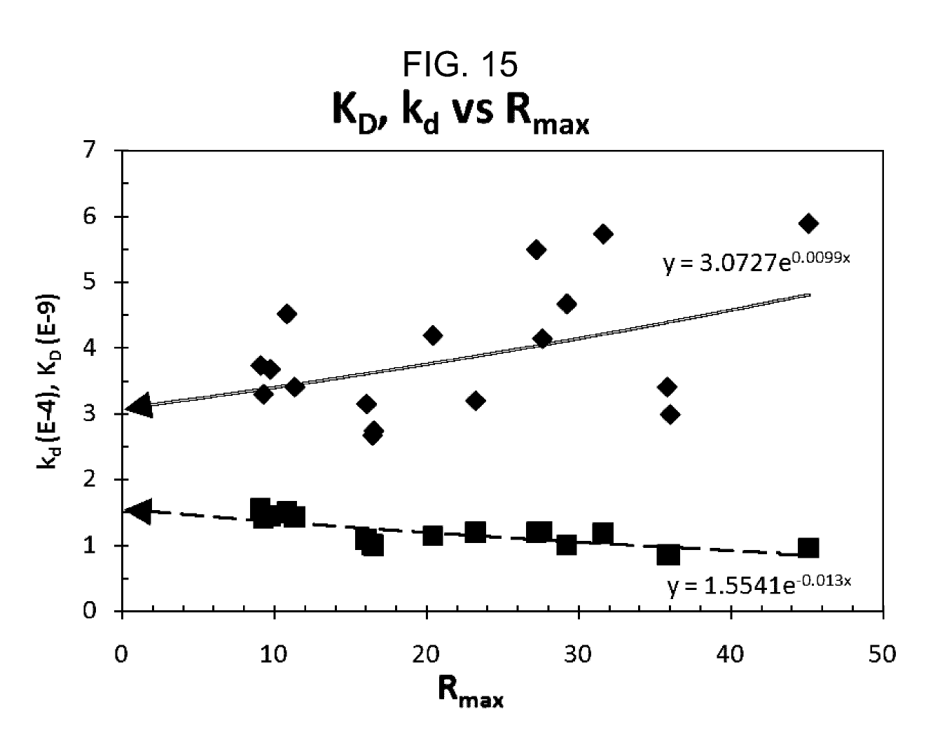

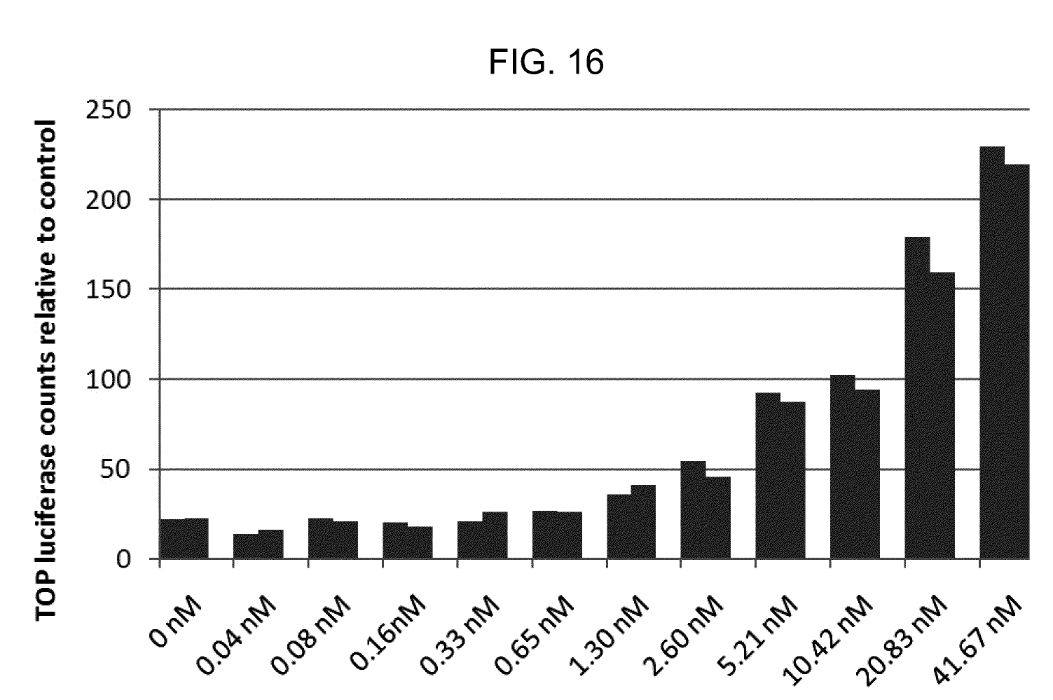

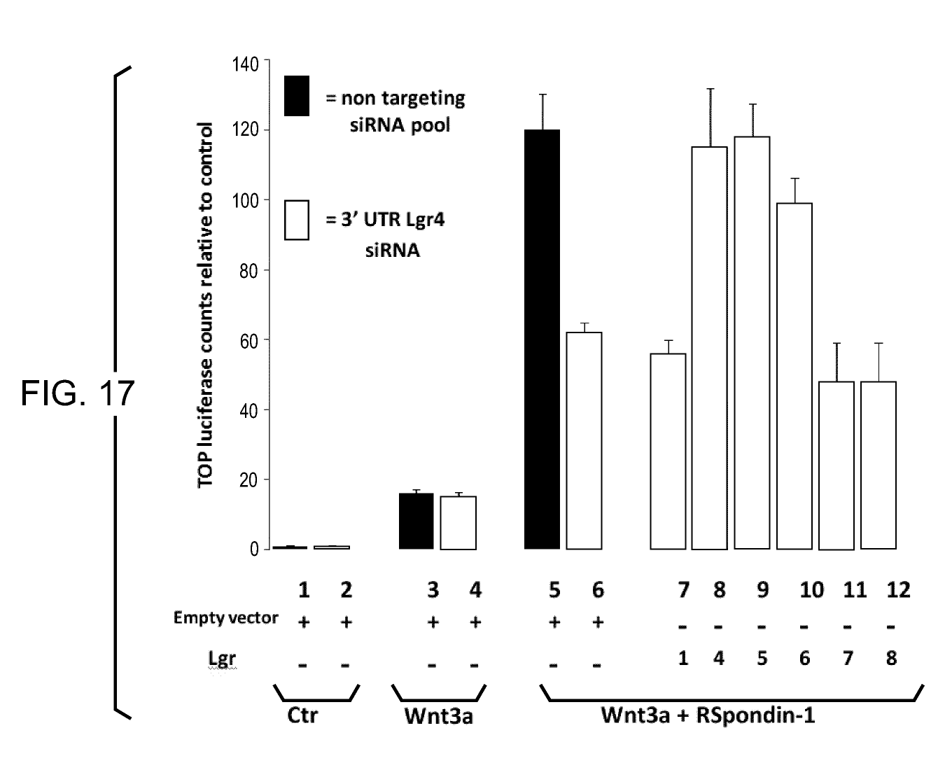

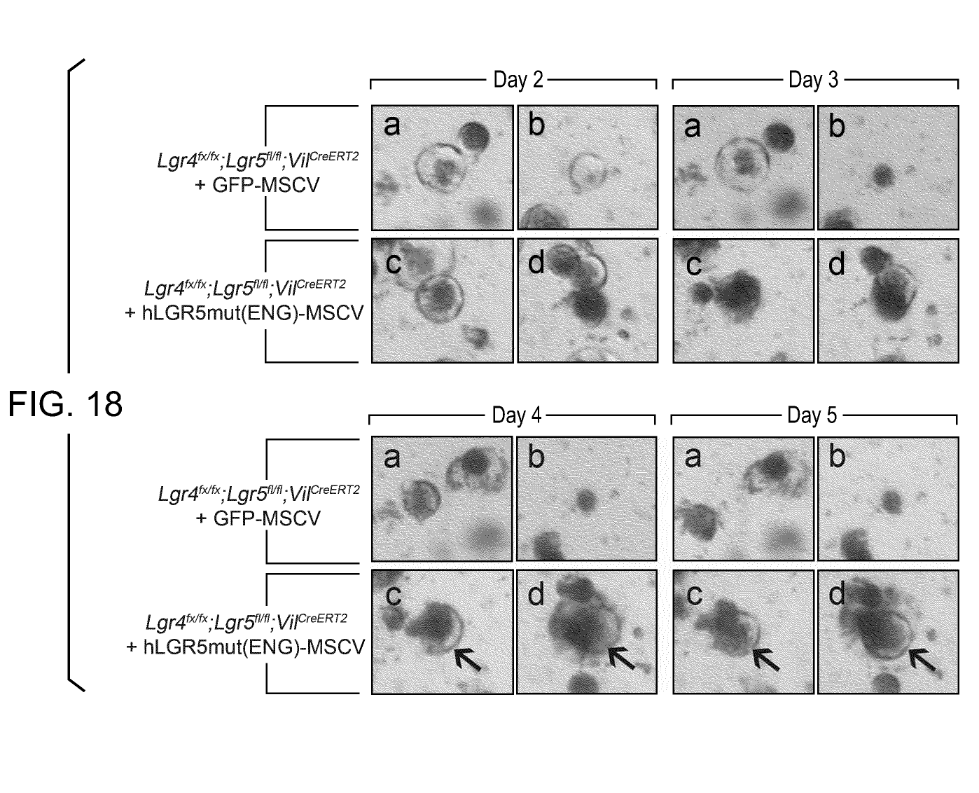

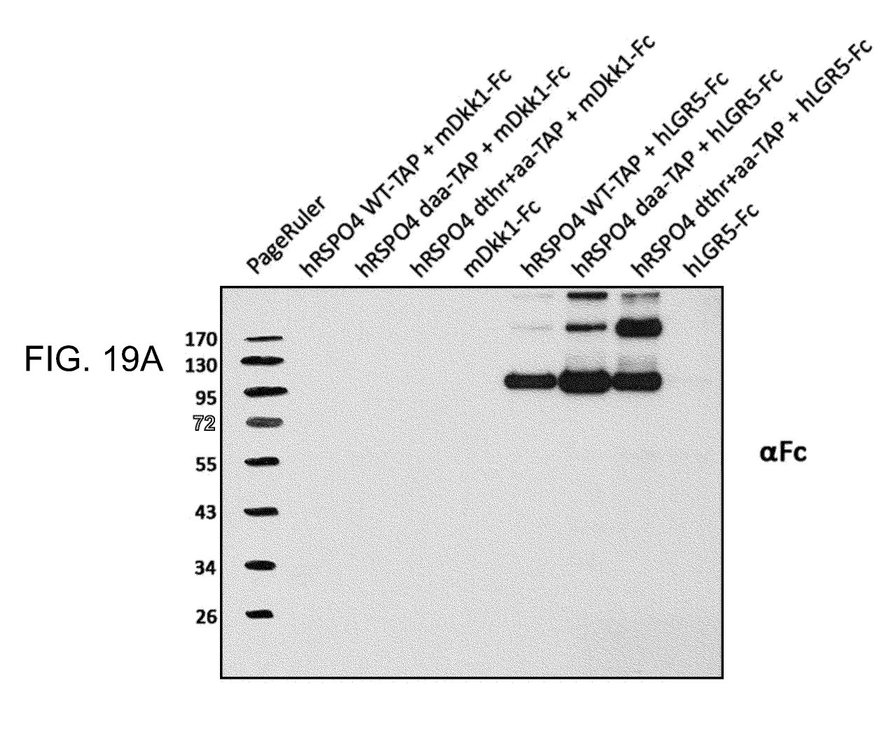

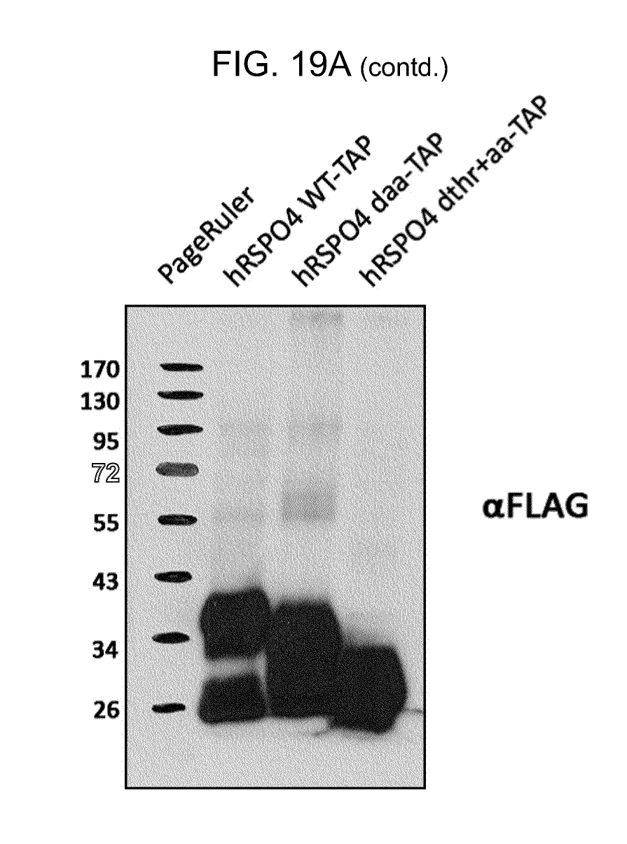

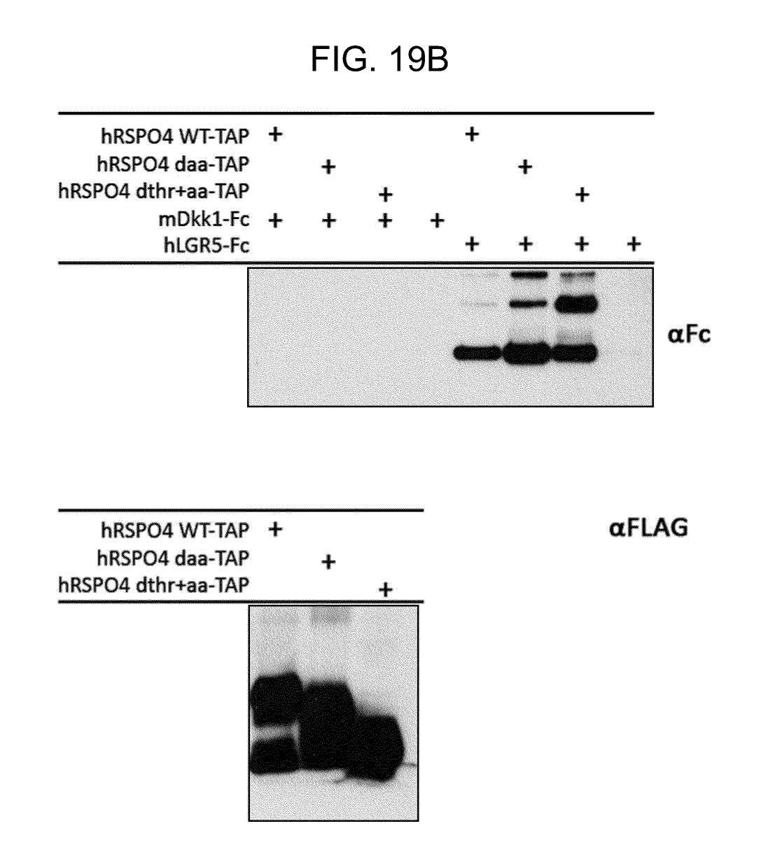

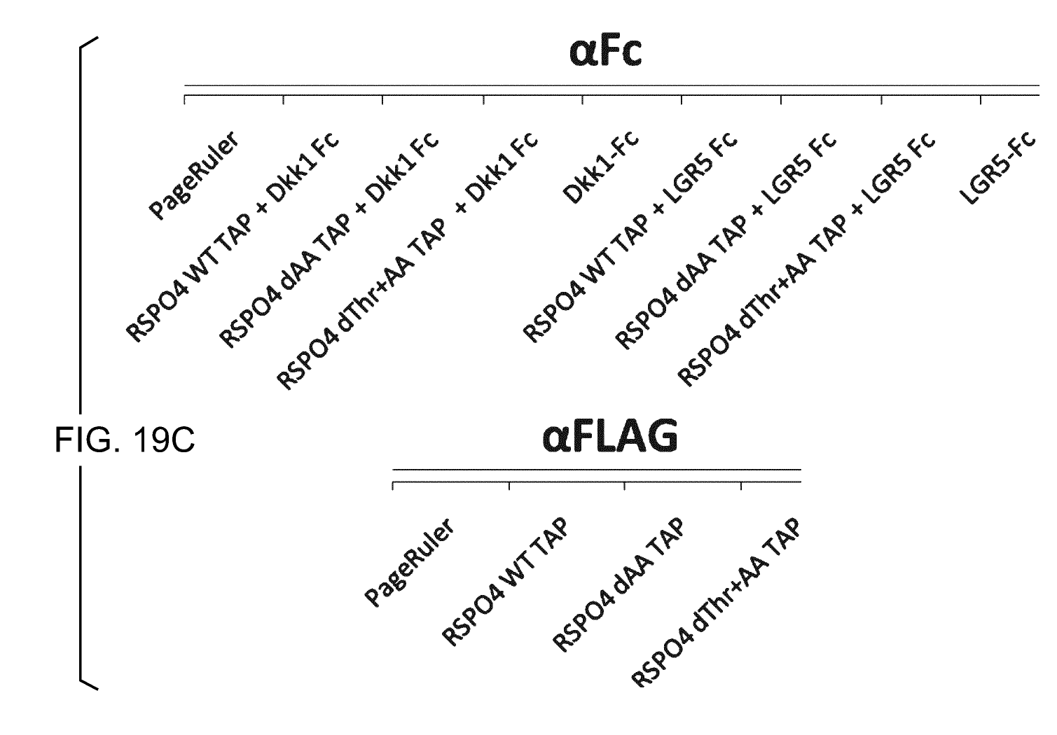

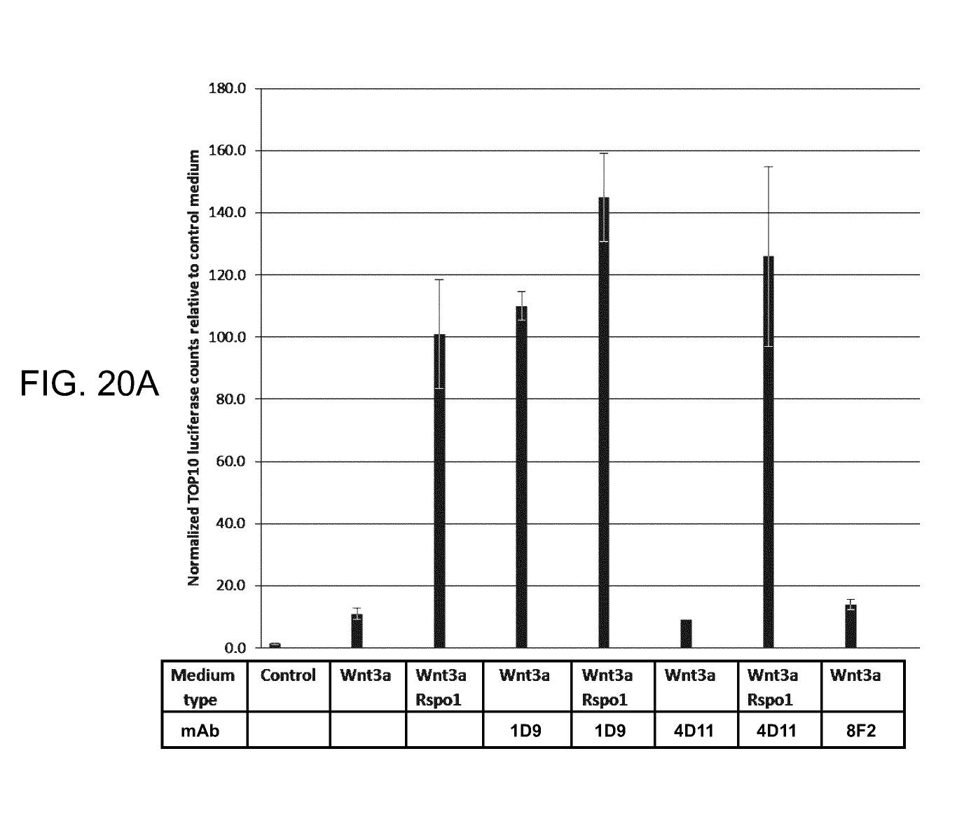

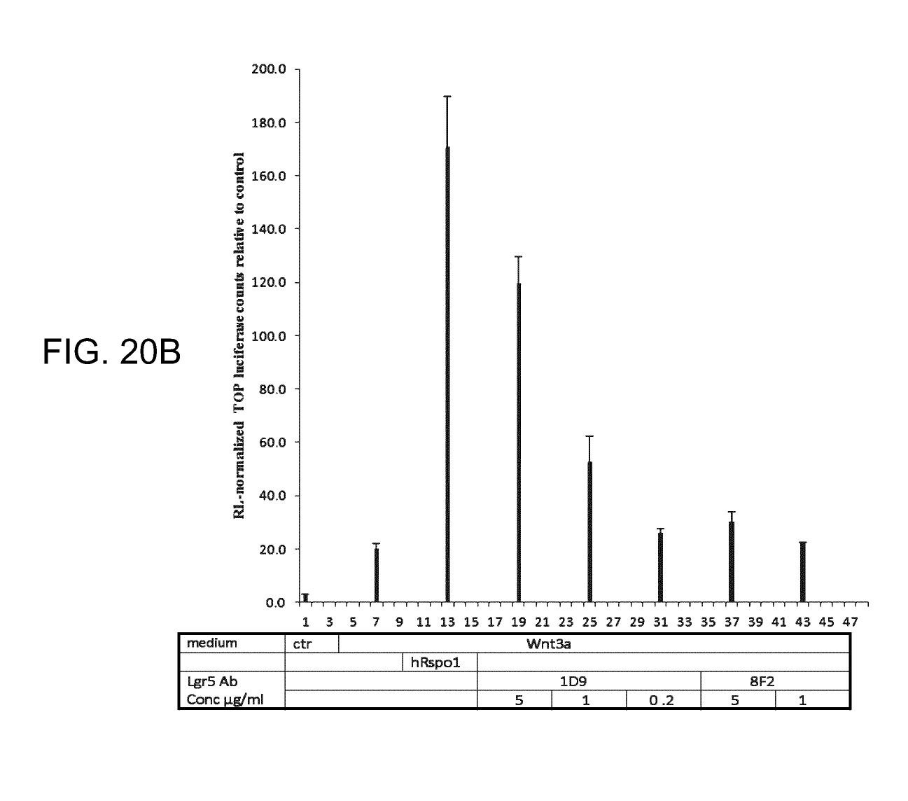

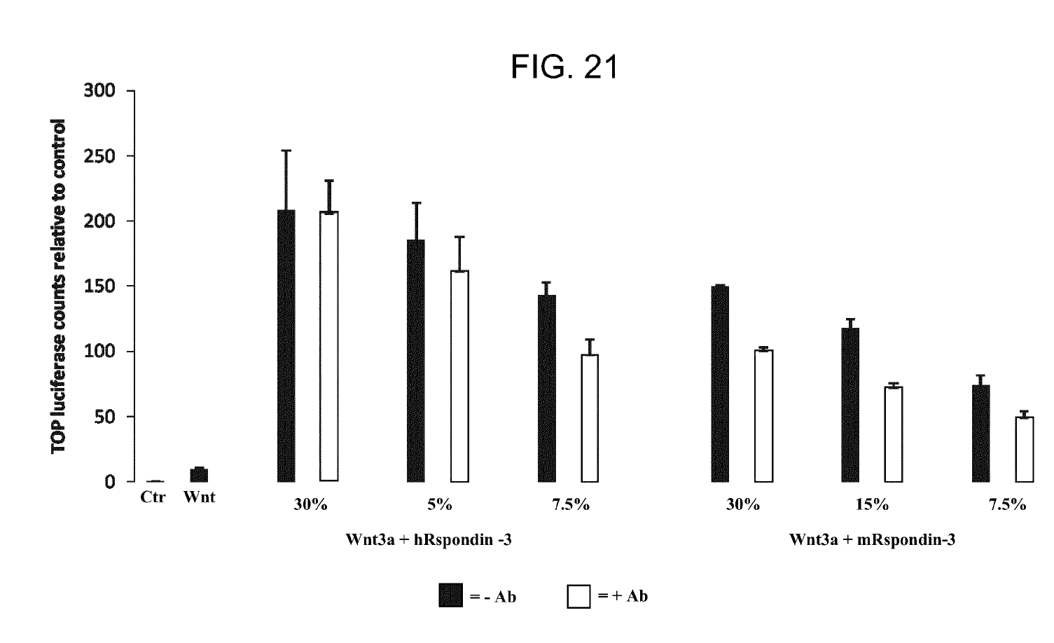

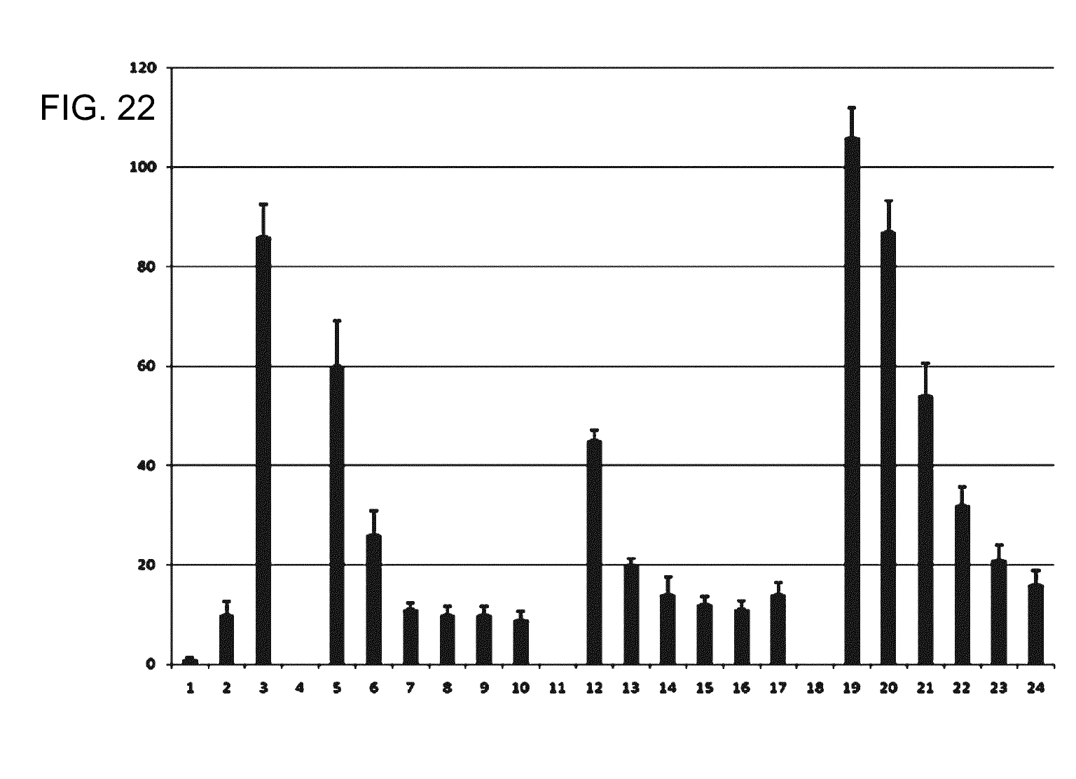

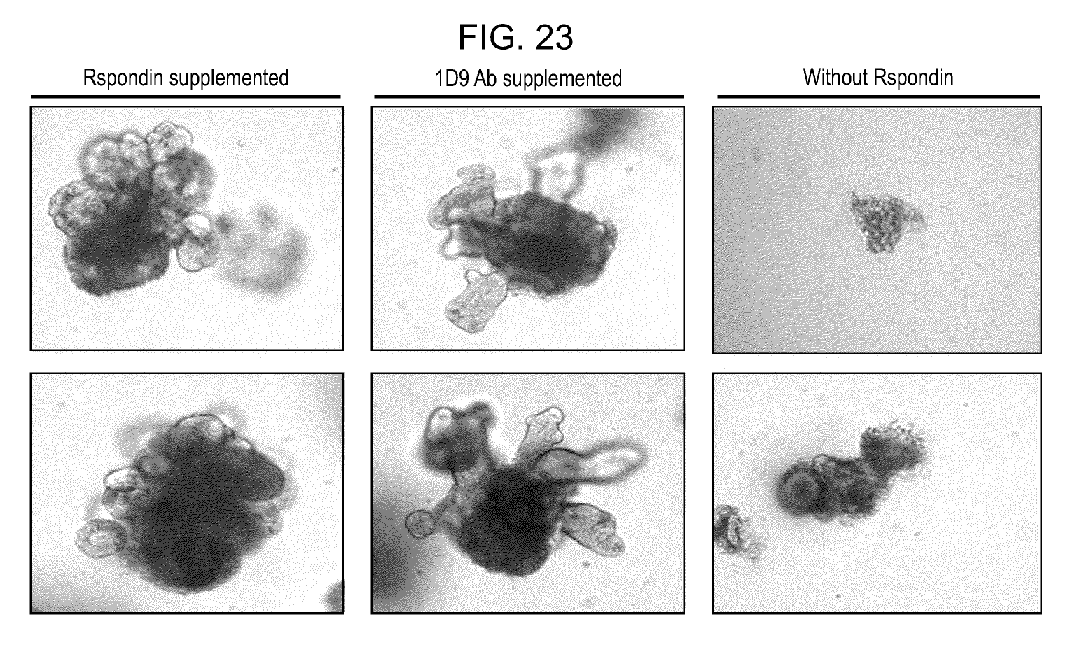

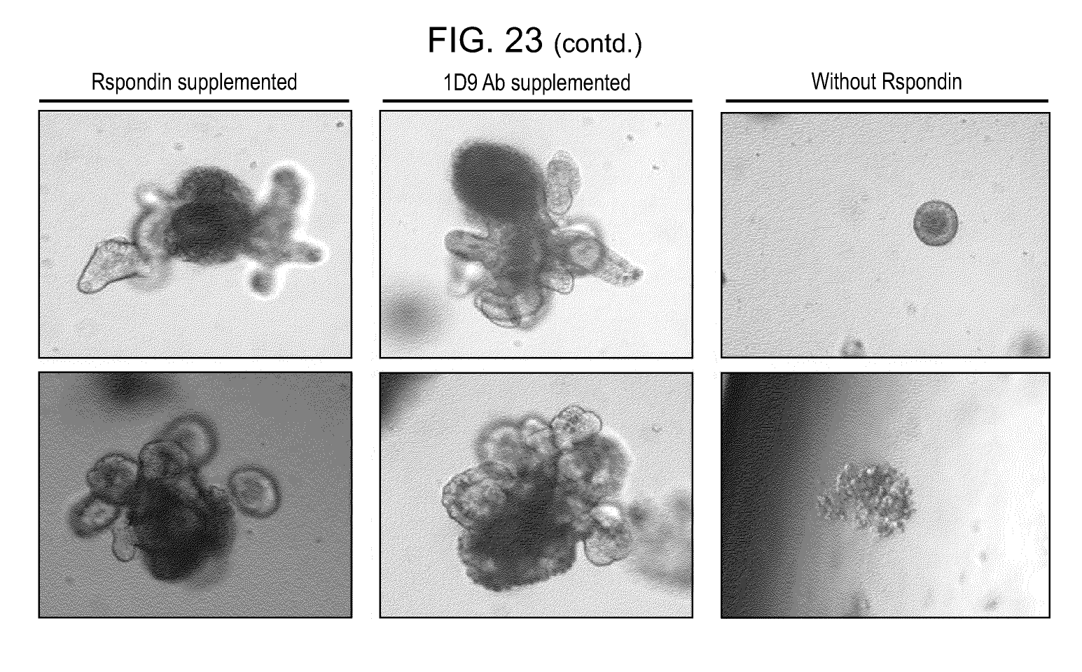

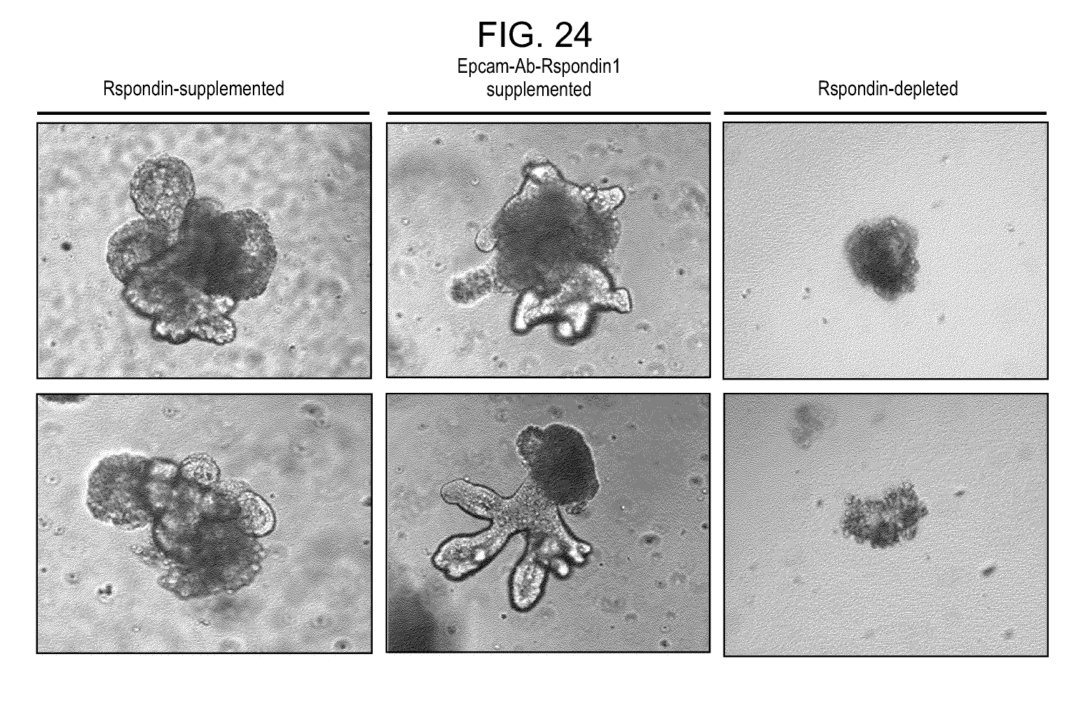

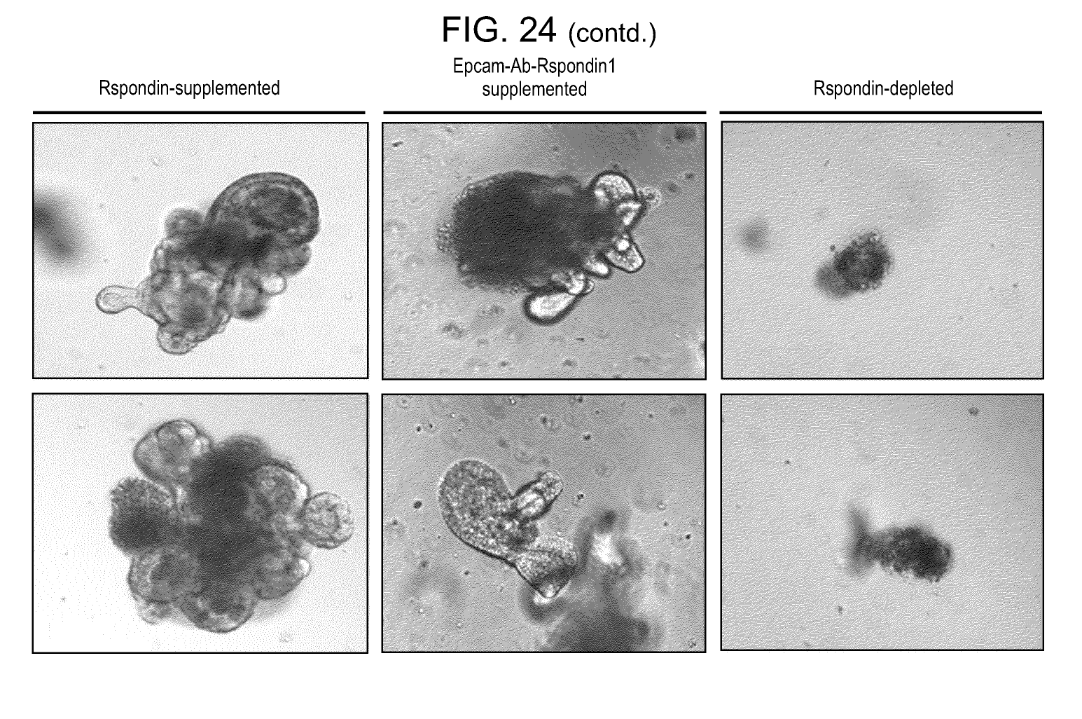

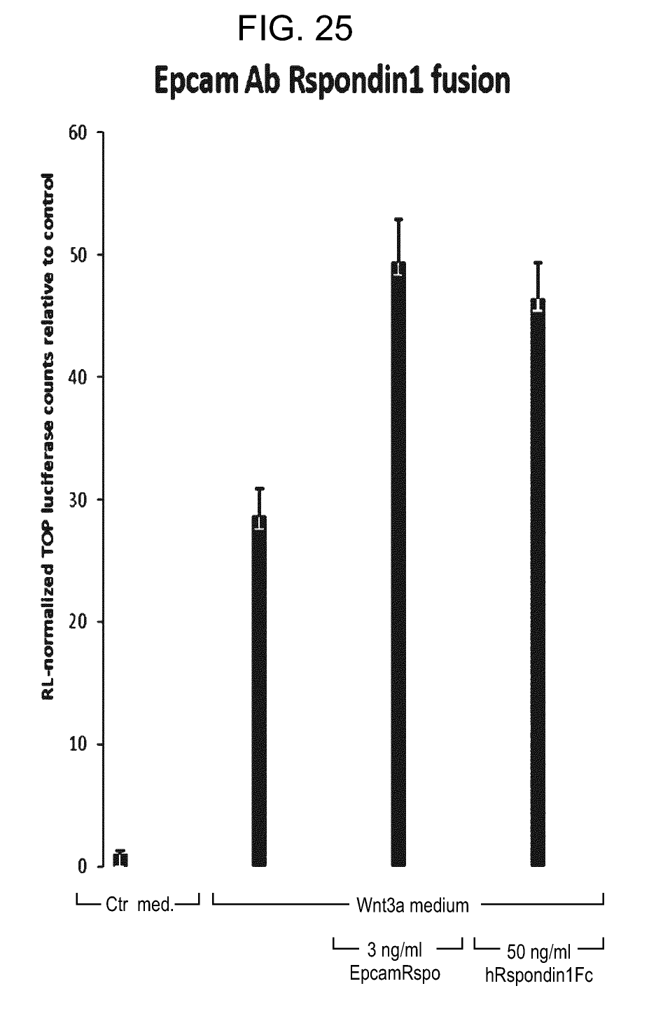

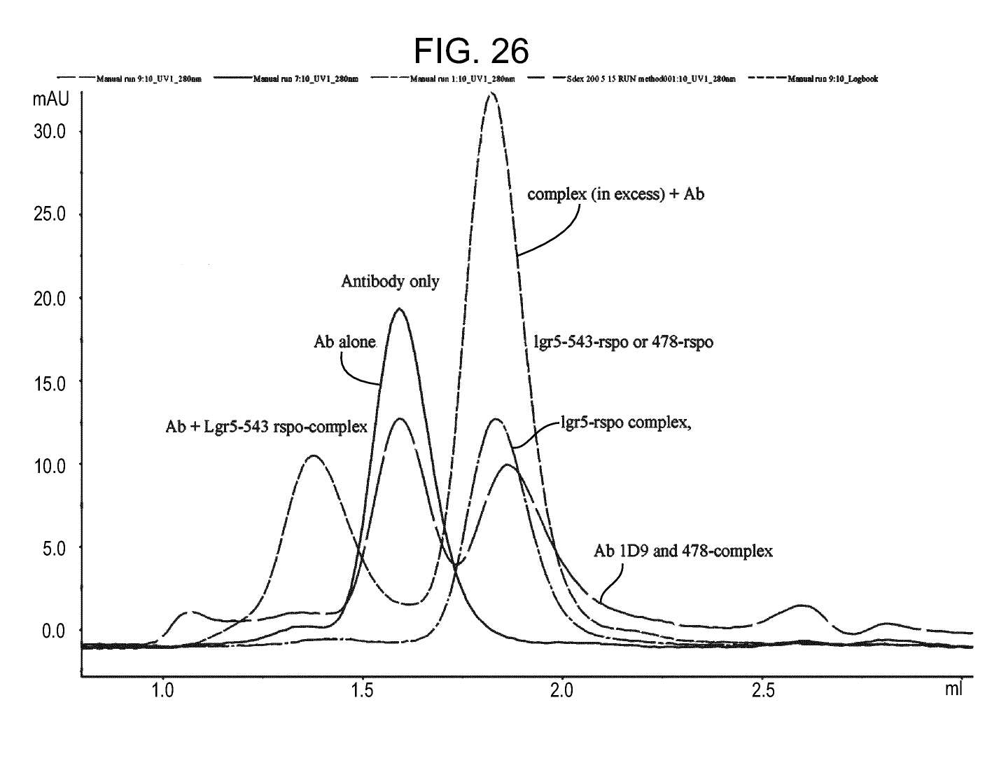

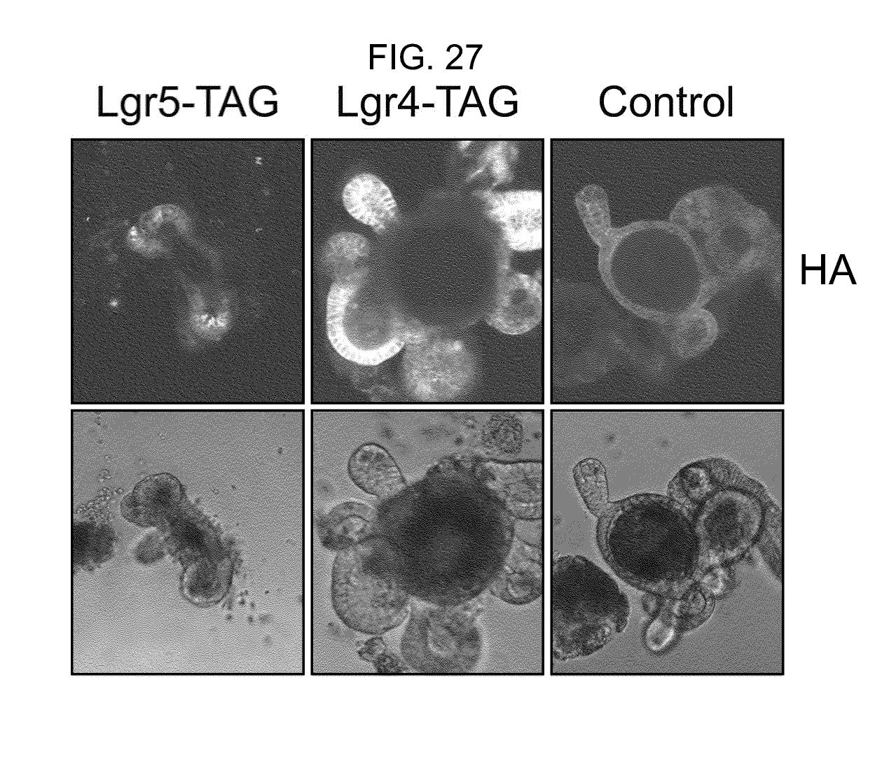

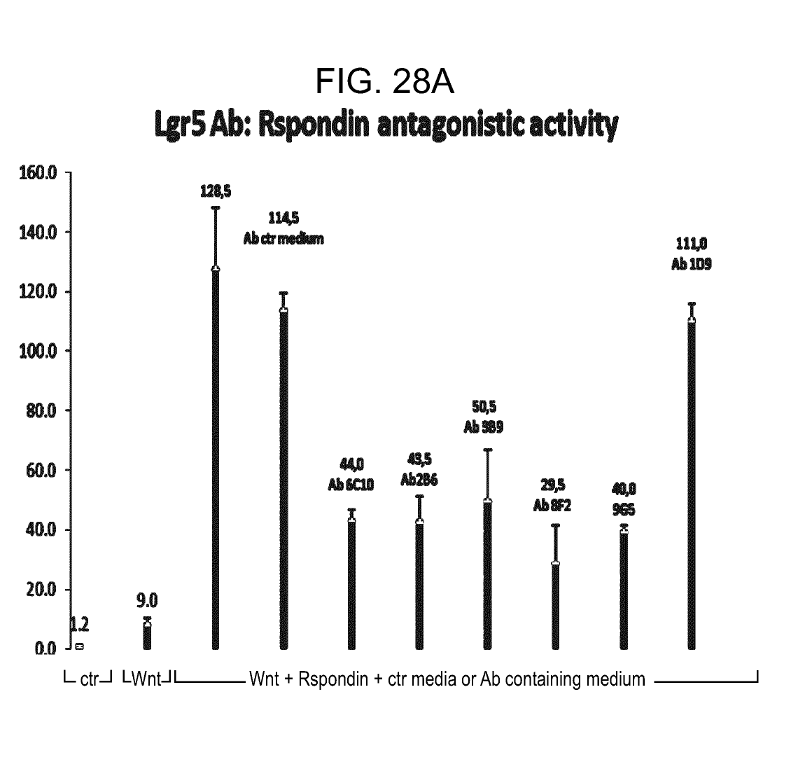

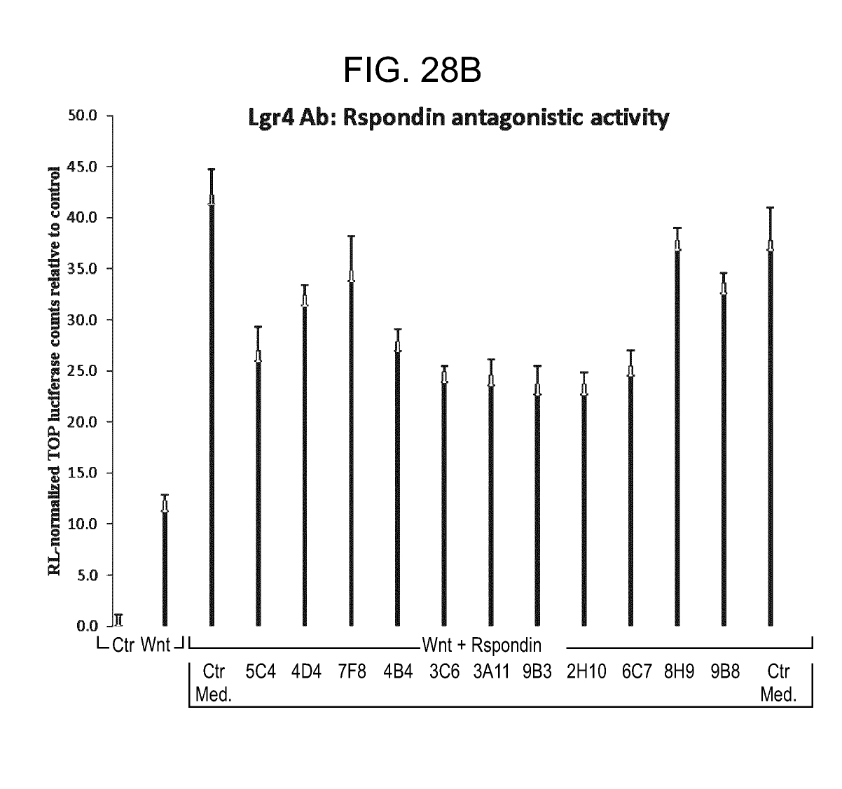

FIGS. 7-18 further support the findings described in Example 1. For further information refer to De Lau et al. Nature, 476, 293-297, 2011.

### Materials and Methods for Example 1

### Microarray Analysis

### Lgr4/5 Knock-Out Mice

Small intestinal crypts were isolated 1 day after induction of deletion from AHCre

Lgr4^{fx/fx} Lgr5^{fl/fl} as well as wt mice by incubation in 2 mM EDTA. Intestines were cut open along the length and villi were removed by scraping with a glass slide (Starfrost microscope slides, 76×26 mm, Waldemar Knittel Glasbeabeitungs GmbH, Germany). The intestines were cut into pieces of approx. 3 cm and washed twice with ice cold DPBS (Gibco 14190). The intestinal fragments were then incubated in DPBS (Gibco 14190) supplemented with 5 mM EGTA (Sigma-Aldrich E4378) for 30 minutes incubation at 4° C. After shaking, the intestinal crypts are in the supernatant. Optional; the intestines were transferred to fresh PBS/EGTA and the procedure was repeated. The supernatant fractions containing the crypts were combined and spun for 5 minutes at 41 g to collect crypts. RNA was isolated from purified crypts using Trizol (Invitrogen, 15596-018) according to the manufacturer's procedures and 1 μg of RNA was labeled using Quick Amp Labelling Kit, two colour using Quick Amp Labeling Kit (Agilent, 5190-0444) according to the manufacturer's procedures (Manual: version 5.5 Feb. 2007) with Cy5 and Cy3, respectively; all as described elsewhere¹⁰³. Two separate biological replicates were performed in dye-swap, resulting in 4 individual arrays. Labeling, hybridization, and washing were done according to Agilent guidelines. 825 ng of differentially labeled cRNA product was hybridized to 4×44K Agilent Whole Mouse genome dual color arrays (G4122F). Array data were normalized and retrieved using Feature Extraction (V.9.5.3, Agilent Technologies) and data analyses were performed using Microsoft Excel (Microsoft Corporation, Redmond, Wash., USA). Features were flagged, if signal intensities for both the Cy3 and Cy5 channel did not pass the Feature Extraction Filter “Significant and Positive” or “Well above Background”. Genes were considered downregulated if 4/4 arrays showed a significant (p<0.05) downregulation of <−0.58 (linear−1.5 fold). This resulted in 379 entries, which 307 unique genes (Table 2: Array data will be available at Gene Expression Omnibus (http://www.ncbi.nlm.nih.gov/geo).

### Rspondin1 Withdrawal in Mouse Intestinal Organoids

Crypts were isolated from a wild type mouse small intestine by incubating with 2 mM EDTA in PBS for 30 min at 4° C., and subsequently cultured in the crypt culture medium as reported previously¹¹⁶. Briefly, isolated crypts were cultured in Matrigel (BD Bioscience) in 24-well plates, and advanced DMEM/F12 medium (Invitrogen) containing EGF, Noggin and Rspo-1 was added after polymerization of Matrigel. Confluent organoids were split into multiple wells, and were then cultured in crypt culture medium in the presence or absence of Rspo-1. One day after Rspo-1 withdrawal organoids (−RSpol organoids) and the control organoids (+Rspo1 organoids) were then collected for RNA extraction and microarray analysis. One μg of RNA from control and Rspo-1 depleted organoids, together with universal mouse reference RNA (Strategene) was labeled using Quick Amp Labelling Kit, two colour (Agilent Technologies) with Cy5 and Cy3 respectively. Samples were hybridized to 4×44K Whole Mouse Genome Microarrays (Agilent, G4122F) according to manufacture's instruction. Microarray signal and background information were retrieved and normalized using Feature Extraction program (V.9.5.3, Agilent Technologies). Samples were considered as well-measured when the fluorescent signals in red channel (Cy5) in either of the samples were greater than 2 fold above the local background. Differences between −Rspo1 organoids and +Rspo1 organoids were calculated by subtracting the ratio for “—RSpol organoids vs reference RNA” from “+Rspo1 vs reference RNA”. Array data will be available at Gene Expression Omnibus (http://www.ncbi.nlm.nih.gov/geo) upon publication.

### Heatmap and Gene Set Enrichment Analysis

Heatmaps were generated using MeV (Multiple Experiment Viewer v.4.3) (Saeed A I, Biotechniques, 2003). Heatmaps were generated using the 306 genes from the Lgr4/5 gene set and plotting the ratios of two different experiments for these genes. FIG. 2A contains the ratios from¹¹⁵, where the authors deleted Apc in the AhCre Apc^{fl/fl} mice and performed microarray analysis 3 days after deletion. FIG. 2B contains the ratios form the Rspo-1 withdrawal experiment described above. Gene set enrichment analysis (GSEA) implemented with GSEA-P v.2.0 (http://www.broad.mit.edu/gsea) was used to identify significant enrichments of the Lgr4/5 gene set in the two different experimental scenarios mentioned above. All “well-measured” features (n=20844) for the Rspo-1 experiment from the Agilent arrays and all features with a ratio (n=45101) for the Apc arrays from the Affymetrix platform were used for the Ranked gene list in GSEA.

### Mass Spectrometric Analysis.

The samples were subjected to SDS-PAGE followed by Coomassie blue staining. The gel lane was sliced into 24 equal sections and subjected to digestion. In brief, protein reduction and alkylation was performed with DTT (60° C., 1 hour) and Iodoacetamide (dark, RT, 30 min), respectively. Digestion was performed with trypsin over night at 37° C. Peptides were extracted with 10% FA¹³⁰.

The extracted peptides were subjected to nanoscale liquid chromatography tandem mass spectrometry (nanoLC-MS/MS) analysis, performed on an Agilent 1200 HPLC system (Agilent technologies) connected to an LTQ Orbitrap Velos (ThermoFisher, Waltham, Mass.)¹³¹. The nanoLC was equipped with a 20 mm×100 μm i.d. trap column and a 400 mm×50 μm i.d. analytical column (Reprosil C18, Dr Maisch, Ammerbuch-Entringen, Germany). Trapping was performed at a flow of 5 μL/min for 10 min and the fractions were eluted using a 45 min linear gradient from 0 to 40% solvent B (0.1 M acetic acid in 80% ACN (v/v), in which solvent A was 0.1 M acetic acid), 40 to 100% solvent B in 2 min and 100% B for 2.5 min. The analytical flow rate was 100 mL/min and the column effluent was directly introduced into the ESI source of the MS using a standard coated fused silica emitter (New Objective, Woburn, Mass., USA) (o.d. 360 μm, tip i.d. 10 μm) biased to 1.7 kV. The mass spectrometer was operated in positive ion mode and in data-dependent mode to automatically switch between MS and MSMS. The five most intense ions in the survey scan were fragmented in the linear ion trap using collision induced dissociation¹³². The target ion setting was 5e5 for the Orbitrap, with a maximum fill-time of 250 ms. Fragment ion spectra were acquired in the LTQ with an AGC value of 5e3 and a max injection time of 100 ms.

Protein Identification: Raw MS data were converted to peak lists using MaxQuant version 1.0.13.13¹³³. Spectra were searched against the IPI (International Protein Index) Human database version 3.37 (69,164 sequences; 29,064,824 residues) using Mascot search engine (version 2.3.02; www.matrixscience.com), with trypsin set as enzyme. The database search was made with the following parameters set to consider a peptide tolerance of ±15 ppm, a fragment tolerance of ±0.5 Da, allowing 2 missed cleavages, carbamidomethyl (C) as fixed modification; oxidation (M) and protein N-terminal acetylation as variable modifications.

### Plasmon Surface Resonance

A pre-activated ester sensor chip (IBIS technologies, Enschede, The Netherlands) was spotted using a Continuous Flow Microspotter (Wasatch Microfluidics (Salt Lake City, Utah, US). In total, 32 spots were created with both mouse antiFLAG, goat antihuman IgG in 8× serial dilution (start concentration is 500 ug/ml and 100 ug/ml respectively) in 10 mM IVIES buffer pH 5.5. Control (reference) spots contained HSA, antiHSA and IVIES buffer. After preparing the sensorchip, the slider was positioned in the instrument IBIS MX96 (IBIS Technologies, Enschede, The Netherlands) for label free Surface Plasmon Resonance (SPR) array analysis. The instrument enables multiplex interactions up to 96-plex using scanning imaging optics for automated calculation of the SPR-dip shifts of all Region of Interests simultaneously. The signal to noise ratio of the instrument which reflects the limit of detection is better than 1 RU corresponding to 1 picogram protein per square mm. In the IBIS MX96, back and forth mixing is applied enabling minimal use of sample while the length of the exposure of the sample to the microarray is unlimited and not affected by the volume of e.g. an injection loop. A two step interaction process was carried out and the multiplex interaction event to all spots of the array was recorded simultaneously. In this way, chip to chip and sample to sample variations can be excluded, while positive- and negative controls and referencing can be applied instantly. A microscope image of the sensor chip (not shown here) can reveal any irregularities, inhomogeneities of the spots and/or disturbing air-bubbles. First 70 microliter of RSPO1-FH was injected followed by dissociation and second injection of 70 μl LGR5-Fc. After regeneration with acid buffer (Gly-HCl, 10 mM, pH=2.0) for 120 seconds, the control experiment was capture of RSPO1-FH followed by injection of Noggin-Fc (results see FIG. 4B). For affinity constant determination first RSPO1-FH was captured on the anti-Flag spot until saturation of the spot followed by injection of LGR5-Fc (8 ug/ml) The RSPO1 was not immobilized directly on the chip because RSPO1 did not survive the acid regeneration process (data not shown). Capturing of RSPO1-FH was possible on the anti-Flag antibody spot from growth medium. FIG. 1 is an overlay plot of three interaction series of first RSPO1-FH (2 minutes association was sufficient to reach saturation) and three different concentrations of LGR5-Fc (Mw 176 kD) corresponding to 45 nM, 23 nM and 11 nM injections. Referencing was carried out by subtraction of the anti-FLag spot signal with the signal coming from a HSA loaded spot in SPRint software (IBIS Technologies, Enschede, The Netherlands) for compensating bulk refractive index shifts e.g. as a result of growth medium compounds. The affinity constant was calculated using a discrete 1:1 interaction model using global fitting (Scrubber 2, BioLogic Software Pty Ltd, Australia). In FIG. 6, the residual plot of the experimental curve minus fit values is shown and although it cannot be revealed that the interaction process is according to a discrete 1:1 interaction model the affinity constant was calculated to be ˜3 nM. It was not necessary to add an extra fit parameter for mass transport limitation compensation, due to a high back and forth mixing condition in the IBIS MX96 flow cell.

### Large-Scale Purification

Transfection and Harvest: cells were cultured as previously described in a number of 15 cm plates according to the number of cells required. Typically, 40 plates were used for 4-5 tandem affinity purifications (TAPs): 40×15 cm plates for Lgr4&5 TAP (LS174T cells); 40×15 cm plates for Frz5 TAP (LS174T cells); 80×15 cm plates for hRspo1 TAP (HEK293T cells); 80-15 cm plates for DKK TAP (HEK293T cells). 40×15 cm plates of 4 or 5 TAP's were grown to 80% full. The cells were collected into 50 ml conical flasks and washed twice with PBS at room temperature (RT). Cells were spun for 5 min. at 1200 rpm (Centrifuge 5810R, Eppendorf). The supernatant was aspirated and the cells were frozen in dry ice at −80° C. All further steps are carried out at 4° C.

### Lysis

the lysis buffer was prepared with (LBI) and without (LB) inhibitors in a clean bottle (LBI in 0.5% NP40). The pellet was resuspended in 40 ml of LBI and incubated on ice for 1 hour. The tube was gently inverted every 6-7 min without vortexing. Following incubation, the tube was spun for 30 min at 20,000 rpm at 4° C. (Centrifuge Avanti J-30I, Rotor JA 25.50, Beckman Coulter). The supernatant, containing cell lysate free from cell membranes, was transferred into a new tube and kept on ice.

### Filtration

approximately 5×0.22 uM filters (Whatmann FP 30/0,45 CA-S Ref. No. 10462100) were prepared along with a 20 ml syringe for each purification. The plunger removed, the filter attached, the lysate loaded (not to the top), the plunger inserted and the lysate filtered into a new tube. Typically, about 2-3 filters were needed for each purification. This step removes other debris from the cell lysate and the volume of the filtered lysate will be slightly lower than the initial volume.

### Preclearing:

850 μl/2 Immunoprecipitations (IPs) were transferred into a 15 ml tube using a cut blue tip. 10 ml of LBI was added and the tube was inverted 6-7 times followed be spinning for 2 min at 1200 rpm. The supernatant was aspirated and a LBI was added in a 1:1 ratio of beads:LBI. It was mixed to resuspend well. 400 μl slurry was transferred into each of the two cell lysate tubes and the tubes were incubated for 60 min. on a wheel (Test-tube rotator Snijder 34528) at 4° C. To remove the protein-G-agarose from the precleared lysate the tubes were spun at 3000 rpm 2′ (Centrifuge 5810R, Eppendorf) and the supernatant was collected. Filter sample with a 0.22 filter.

### FLAG IP:

a volume (500 μl/2 IP's) of M2 FLAG resin (anti-FLAG M2 affinity gel, Sigma A2220-5ML) was transferred to a 15 ml conical tube using a cut blue tip. 10 ml of LBI was added and gently mixed by inverting the tube 6-7 times followed by spinning for 2 min at 1200 rpm. The supernatant was aspirated and resuspended in the resin at a 1:1 ratio of beads:LBI. 250 μl slurry of M2 FLAG resin was added to each sample. This was incubated for 2 hours on a wheel in a cold room. The product of these steps was IPs of anti-FLAG bound to FLAG portion of double-tagged proteins (FLAG-HA-Tagged).

### Washes:

samples were spun at 1200 rpm for 2 min and the supernatant removed by aspiration. The wash was achieved by adding 10 ml of LBI and mixing by inverting the tube gently 6-7 times and spinning at 1200 rpm for 2 min. Four more washes were performed in 1.5 ml eppendorf tubes using 1 ml of LBI each time.

### FLAG Elution (Performed at Room Temperature):

after the last wash, the supernatant was aspirated (not completely) and 1.2 ml of the diluted 3×FLAG peptide (100 ug/ml) was added per IP. A dilution of 3×FLAG peptide (Sigma F4799-4MG) in LBI: [stock]=2 mg/ml; [final]=100 μg/ml. 125 μl stock of 3×FLAG peptide was purified in 2.5 ml of LBI (changing the volume where required). This was followed by incubation on the wheel for 20 min at room temperature. The resulting product of these steps was FLAG-HA-tagged protein without anti-FLAG; antiFLAG is now bound to FLAG-peptide which was added in excess; the complexed beads anti-FLAG-FLAG peptide were then removed by the centrifuge step in the HA IP steps below.

### HA IP (All Steps at 4° C.):

the sample was spun at 3000 rpm twice, then purified on a Millipore Millex GV4 0.22 um filter. 120 ul/IP slurry of HA resin (anti-HA affinity matrix Rat monoclonal, ref 11815016001, Roche) was transferred into 15 ml tube using a cut yellow tip. 10 ml of LBI was added and mixed by gently inverting the tube for 6-7 times. The resin was spun down 1200 rpm for 2 min and the supernatant removed by aspiration. The resin was resuspended to a ratio of 1:1 beads:LBI. The eluate was added to the HA resin and the tube containing the HA IP was incubate on the wheel for 2 hours at 4° C. These steps provided immunoprecipitates comprising of anti-HA bound to HA portion of double-tagged proteins (FLAG-HA-Tagged).

### Washes

after removing the supernatant, two washes (1 ml each) were carried out in LBI, gently inverting the tube 6-7 times. This was followed by spinning at 1200 rpm for 2 min and 3 further 1 ml washes in LB.

### HA Elution

the samples were resuspended in 150 μl of LB and transferred into one spin column filter (Biorad micro biospin column catno. 732-6204) and collection tube. Each sample was spun at 1400 rpm for 1 min. The filter containing the beads was transferred onto a new clean eppendorf tube and 50 μl of 2× Laemmli sample buffer (Sigma, S3401-10VL) were added to the beads. The tube was incubated for 1 min and spun at 1400 rpm for 1 min. This was repeated for additional sequential elutions. The samples were then frozen at −80°. The eluted product was a double tagged protein without anti-HA. (Laemmli sample buffer denatures the anti-HA antibody and therefore the anti-HA is removed from the FLAG-HA-tagged protein. The remaining bait protein plus its captured proteins is what is left.)

Silver Staining with SilverQuest™ Silver Staining Kit (Cat #LC6070, Invitrogen):

10 μl of each sample (HA elution) were loaded on a 1 mm 4-12% NuPAGE Bis-Tris Gel (cat #NP0321BOX, Invitrogen). 5 μl of the FLAG elution (total sample 200 μl) were loaded followed by 2 μl of marker and 5 μl of 2× sample buffer. The BASIC protocol of the SilverQuest™ kit was followed. The steps were carried out in a 50 ml volume using 50 ml conical tubes (instead of 100 ml).

### Lysis Buffer (LB):

### 50 mM Tris HCl pH 7.4

### 150 mM NaCl

### 1 mM MgCl₂

### 10% Glycerol

### 0.5% NP40

### 1 mM CaCl₂

### H₂O (MilliQ)

### Lysis Buffer+Inhibitors (LBI):

50 ml LB+1 tablet protease inhibitor cocktail (Complete EDTA-free Protease Inhibitor Cocktail tablet; Cat no 11873580001, Roche Applied Science).

### Example 2

### The Furin Domain of Rspondin is Sufficient for Binding and Rspondin Activity

The inventors generated truncated Rspondin fragments including: (i) deletion of the basic amino acid domain (hRSP4 daa) (SEQ ID NO: 139) and (ii) deletion of the thrombospondin domain and basic domain (hRSPO4 dtht+aa) (SEQ ID NO: 140). These truncated Rspondin proteins, and Wild type full-length Rspondin (hRSPO4) as a control, were used to precipitate hLgr5 (Fc-tagged) and DKK (Flag-tagged). It was found that DKK does not bind to hRSPO4 or any of the fragments of hRSPO4. By contrast, hLgr5 coimmunoprecipitated with full-length Rspondin, and also with both Rspondin fragments. The fragment hRSPO4 dtht+aa represents the Furin domain of Rspondin4 (both the thrombospondin and basic domains have been deleted) (see FIG. 19). Therefore, the results show that the Furin domain is sufficient for binding to Lgr5.

The inventors have also shown that an anti-Rspondin 3 monoclonal antibody, which binds to epitopes in the Furin domain of Rspondin 3, blocks the Wnt-enhancing activity of Rspondin (for example, see FIG. 21). HEK293T cells were transfected with Wnt pathway-specific (TOP) luciferase reporter in combination with a TK driven Renilla reporter. After 24 hrs cells were incubated for 24 hrs with control conditioned medium (CM), Wnt3a CM, or combinations of Wnt3a+Rspondin-CM. The relative volume proportion (%) of Rspondin CM is indicated in FIG. 21. A rat monoclonal antibody (R&D systems, clone 400403), recognizing the furin domain of both human and mouse Rspondin3, was added to a final concentration of 2.5 μg/ml. Wnt pathway activity was quantitated by comparing averages of triplicate Renilla-normalized TOP luciferase counts of cells receiving stimuli, to un-stimulated cells. FIG. 21 shows that the TOP luciferase counts (i.e. Wnt pathway activity) is lower when the anti-Rspondin-3 antibody is added to the assay. The effect is greater when a lower proportion of Rspondin is used in the assay. Therefore, the inventors conclude that the Furin domain is sufficient for binding and also necessary for the activity of Rspondin.

In another experiment, the inventors tested the activity (in terms of Wnt enhancement) of the Rspondin fragments (as described above) in a TCF reporter assay. FIG. 22 shows the results of this assay. Columns 19-20 show luciferase counts for the furin domain fragment of Rspondin (SEQ ID NO: 140) and demonstrate that the furin domain fragment activates the Wnt pathway to a greater extent than full length Rspondin or the Rspondin fragment comprising thrombospondin and furin domains (SEQ ID NO: 139). This experiment shows that the furin domain is not only necessary but also sufficient for mimicking full-length Rspondin Wnt activation. Furthermore, it suggests that the furin domain fragments are more effective at enhancing the Wnt pathway than full-length Rspondin. Therefore, the furin domain fragment is an example of a surprisingly potent agonist of the invention.

### Materials and Methods for Coimmunoprecipitation Experiments (FIG. 19):

M2-beads, αFLAG Staining

1. hRSPO4-WT-TAP (step 2: +hRSPO4-WT-TAP)
2. hRSPO4-dAA-TAP (step 2: +hRSPO4-dAA-TAP)
3. hRSPO4-dTHR + AA-TAP (step 2: hRSPO4-dTHR + AA-TAP)

M2-beads, αFc Staining

1. hRSPO4-WT-TAP (step 2: +Dkk1-Fc)
2. hRSPO4-dAA-TAP (step 2: +Dkk1-Fc)
3. hRSPO4-dTHR + AA-TAP (step 2: +Dkk1-Fc)
4. Dkk1-Fc (step 2: +Dkk1-Fc)
5. hRSPO4-WT-TAP (step 2: +LGR5exo-Fc)
6. hRSPO4-dAA-TAP (step 2: +LGR5exo-Fc)
7. hRSPO4-dTHR + AA-TAP (step 2: +LGR5exo-Fc)
8. LGR5exo-Fc (step 2: +LGR5exo-Fc)

All actions performed on ice or at 4° C.
Cut the top off a blue pipet tip and pipet the appropriate (total) amount of beads needed into a 15 ml tube (11×30 ul=360 ul)
Wash the beads with 10 ml lysis buffer (LB)
Spin down at 1200 rpm for 2 min at 4° C.
Add 11 ml of LB and mix well
Add 1 ml of the mixture to each Eppendorf tube
Spin down for 2 min at 1200 rpm
Remove the LB
Incubate overnight with 2 ml of step 1 CM at 4° C. on a spinning wheel
Wash twice with 2 ml of LB (spin down at 1200 rpm for 2 min at 4° C.)
Transfer to a 1.5 ml Eppendorf tube
Add 1.5 ml of LGR5-Fc CM and incubated at 4° C. on a spinning wheel for four hours
Wash four times with 1 ml of LB
Carefully remove all LB
Add 40 ul of 2× sample buffer was and mix well
Heatshock for 3 min at 70° C.
Spin down at 1200 rpm, 4° C. for 2 min
Load 30 μl on a 10% gel
Load empty lanes with 20 μl of 2× sample buffer
Load 8 μl of a PageRuler™ prestained protein ladder
Run gel at 80 V through the stacking and then at 100 V through the separation gel
Blot overnight at 4° at 100 mA
Block the blots with a 10% milk in TBS solution for 1 h
Stain the blots with 1:2000 αFLAG or 1:5000 αhIgG respectively in 10% milk in TBST
Wash the blots for 45 min with TBST, refresh 5 times

### Example 3

### 1D9 is an Agonist of Lgr5

Several known anti-Lgr5 antibodies were tested for agonistic activity towards Lgr5 using the TCF reporter assay. HEK293T cells stably transfected with LGR5 are transfected with the TOP reporter and a TK driven Renilla reporter constructs. After 24 hours the cells were stimulated with: 1) Wnt3A condition medium (Produced in L-Cells and added at ⅓ of the total medium volume), 2) RSpol His tagged purified on a nickel column from 293T cells added at 1 ug/ml, 3) Anti LGR5Rat monoclonal antibodies 1D9, 4D11, 8F2 prot A purified at 1 ug/ml. Luciferase activity was determined 24 h after stimulation.

The graph in FIG. 20A shows that luciferase count is low in the absence of Rspo1 (less than 20.0), except for where antibody 1D9 is included in the assay. When 1D9 is used in the absence of Rspo1, luciferase activity of approximately 110 is observed, which is similar to the luciferase activity of 100, which is observed when Rspo1 is used in place of 1D9. When both 1D9 and Rspo1 are used together in the assay, Wnt expression is even greater (represented by the luciferase counts of approximately 145). Therefore, 1D9 is an agonist of Lgr5 that mimics the activity of Rspo1.

HEK293T cells were seeded into 96-well plates in DMEM/10% FCS at a density of 10⁴ cells/well and in triplicate transfected (PEI: Polyethylenimine, linear, MW-25,000) with 10 ng TOP or FOP luciferase (Ref 1), 1 ng TK Renilla, 10 ng of pcDNA-based hLgr5-Flag, and 80 ng empty vector DNA. After 24 hrs, medium was replaced for 50% fresh DMEM/10% FCS, and 50% Wnt3a-conditioned medium or control conditioned medium. At the same time point, purified human Rspondin1 (stock solution of 100 μg/ml in PBS) was added at a final concentration of 1 μg/ml and ProtA-purified (stock solution of 1 mg/ml in PBS) Lgr5-specific antibodies. At 72 hrs results were measured using a dual luciferase assay kit (Promega USA).

The graph in FIG. 20B shows that luciferase count is low in the absence of Rspo1 (approximately 20.0—see column 7) compared to luciferase count with Rspo1 and Wnt3a (approximately 170 counts—see column 13). Antibody 1D9 in part compensates for the absence of Rspo1 as is shown in columns 19, 25 and 31. The greater the concentration of antibody 1D9 the greater the luciferase counts, demonstrating that antibody 1D9 is acting as an agonist of the Wnt pathway. Antibody 8F2 was did not show any significant increase in Wnt activity compared to Wnt3a in the absence of Rspondin (see columns 7, 37 and 43).

1D9 binds to the hinge region of Lgr5, more specifically the CRL region represented by SEQ ID NO: 57 (see also FIG. 4C).

### Example 4

### Multi-Targeting Agonists

The inventors generated a synthetic fusion protein, by methods well known in the art, comprising an anti-Epcam antibody linked to an Rspondin1 furin domain fragment (SEQ ID NO: 141 represents the sequence of the full Rspondin1 furin domain fragment used including the flexible linker peptide; SEQ ID NO: 142 represents the sequence of the flexible linker peptide alone; and SEQ ID NO: 143 represents the sequence of the furin repeat region in the fusion protein in the Rspondin furin domain fragment). Epcam (also referred to as TACSTD1 or CD326) is an epithelial cell adhesion molecule which is expressed on most epithelial cells. Epcam is also expressed on the basolateral membrane of the intestine (it faces towards the interstitium, and away from the lumen). The inventors have discovered that Lgr proteins also reside on the basolateral side of the membrane.

By linking the furin domain fragment (an agonist of Lgr proteins—see example 2) to an anti-Epcam antibody, the inventors targeted the furin domain fragment to epithelial cells, and more specifically, to the basolateral side of the epithelial cells, thus generating a bispecific multi-targeting agonist (targeted to and specific for both Lgr and Epcam). To test the Rspondin-mimicking activity of this multi-targeting agonist, the inventors grew mouse colon organoids in Sato medium, which is known to require Rspondin for organoid growth (see Sato et al., Nature 459, 262-5, 2009). The inventors then replaced the Rspondin with the multi-targeting agonist to observe whether the agonist could rescue growth of the organoids in an Rspondin-depleted medium. It was found that, not only could the mouse organoids grow to an equivalent size in the presence of the multi-targeting agonist, but also that the multi-targeting agonist had a similar effect to Rspondin at a 50× lower concentration (see FIG. 24). This demonstrates that the anti-Epcam antibody effectively targets the agonist to epithelial cells and mimics the activity of Rspondin binding to an Lgr protein and enhancing the Wnt pathway, as is required for organoid growth. The fact that the multi-targeting agonist can be used at 50× lower concentrations than Rspondin gives an indication of possible dosage requirements (both for use in therapy and for use in culture media) and also indicates that it could be suitable for use in therapy because lower doses combined with targeted activity would result in fewer unwanted side effects in the patient.

### REFERENCES

¹Reya, T. & Clevers, H. Wnt signalling in stem cells and cancer. Nature 434, 843-850 (2005).
²Van de Wetering, M. et al., The beta-catenin/TCF-4 complex imposes a crypt progenitor phenotype on colorectal cancer cells. Cell 111(2), 241-250 (2002).
³Uchida, H. et al., Overexpression of leucine-rich repeat-containing G protein-coupled receptor 5 in colorectal cancer. Cancer Sci. 101(7), 1731-1737 (2010).
⁴Kazanskaya. O., Glinka, A., del Barco Barrantes, I., Stannek, P., Niehrs, C., & Wu, W. R-Spondin2 is a secreted activator of Wnt/beta-catenin signaling and is required for Xenopus myogenesis. Dev. Cell 7, 525-534 (2004).
⁵Kim, K. A. et al., Mitogenic influence of human R-spondinl on the intestinal epithelium. Science 309(5738), 1256-1259 (2005).
⁶Korinek, V. et al., Depletion of epithelial stem-cell compartments in the small intestine of mice lacking Tcf-4. Nature Genetics 19(4), 379-383 (1998).
⁷Nuttall, M. E., & Gimble, J. M. Is there a therapeutic opportunity to either prevent or treat osteopenic disorders by inhibiting marrow adipogenesis? Bone 27, 177-184 (2000).
⁸Takashima, S., et al., The Wnt agonist R-spondinl regulates systemic graft-versus-host disease by protecting intestinal stem cells. J. Exp. Med. 208(2), 285-294 (2011).
⁹Gat, U., DasGupta, R., Degenstein, L., & Fuchs, E., et al., De Novo hair follicle morphogenesis and hair tumors in mice expressing a truncated beta-catenin in skin. Cell 95, 605-614 (1998).
¹⁰Li, Y. H., Zhang, K., Ye, J. X., Lian, X. H., & Yang, T. Wnt10b promotes growth of hair follicles via a canonical Wnt signalling pathway. Clin. Exp. Dermatol. [Epub ahead of print] (2011).
¹¹WO2010/090513
¹²Niehrs, C., Norrin and frizzled; a new vein for the eye. Dev. Cell 6, 453-454 (2004).
¹²Parma, P. et al., R-spondinl is essential in sex determination, skin differentiation and malignancy. Nature Genetics 38(11), 1304-1309 (2006).
¹⁴Aoki, M., Mieda, M., Ikeda, T., Hamada, Y., Nakamura, H., & Okamoto, H., R-spondin3 is required for mouse placental development. Dev. Biol. 301, 218-226, (2006).
¹⁵Blaydon, D. C. et al., The gene encoding R-spondin 4 (RSPO4), a secreted protein implicated in Wnt signaling, is mutated in inherited anonychia. Nature Genetics 38, 1245-1247 (2006).
¹⁶Wei Q., Yokota, C., Semenov, M. V., Doble, B., Woodgett, J., & He, X., R-spondinl is a high affinity ligand for LRP6 and induces LRP6 phosphorylation and beta-catenin signaling. J. Biol. Chem. 282(21), 15903-15911 (2007).
¹⁷Binnerts, M. E. et al., R-Spondin1 regulates Wnt signaling by inhibiting internalization of LRP6. Proc. Natl. Acad. Sci. USA 104(37), 14700-14705 (2007).
¹⁸Kim, K. A. et al., R-Spondin family members regulate the Wnt pathway by a common mechanism. Mol. Biol. Cell 19(6), 2588-2596 (2008).
¹⁹WO2009/005809
²⁰Barker, N., Clevers, H., Leucine-rich repeat-containing G-protein-coupled receptors as markers of adult stem cells. Gastroenterology 138, 1681-1696 (2010).
²¹Van Schoore, G., Mendive, F., Pochet, R., & Vassart, G., Expression pattern of the orphan receptor LGR4/GPR48 gene in the mouse. Histochem. Cell. Biol. 124, 35-50 (2005).
²²Snippert, H. J. et al., Lgr6 marks stem cells in the hair follicle that generate all cell lineages of the skin. Science 327, 1385-1389 (2010).
²³Molenaar, M. et al., XTcf-3 transcription factor mediates beta-catenin-induced axis formation in Xenopus embryos. Cell 86(3), 391-399 (1996).
²⁴Willert, J., Epping, M., Pollack, J. R., Brown, P. O., & Nusse, R. A transcriptional response to Wnt protein in human embryonic carcinoma cells. BMC Dev. Biol. 2, 8 (2002).
²⁵Jackson, A. et al., Gene array analysis of Wnt-regulated genes in C3H10T1/2 cells. Bone 36(4), 585-598 (2005).
²⁶Chen, S., McLean, S., Carter, D. E. & Leask, A., The gene expression profile induced by Wnt 3a in NIH 3T3 fibroblasts. J. Cell. Commun. Signal 1(3-4), 175-183 (2008).
²⁷Klapholz-Brown, Z., Walmsley, G. G., Nusse, Y. M., Nusse, R. & Brown P O., Transcriptional program induced by Wnt protein in human fibroblasts suggests mechanisms for cell cooperativity in defining tissue microenvironments. PLoS One 2(9), e945 (2007).
²⁸Longo, K. A. et al., Wnt signaling protects 3T3-L1 preadipocytes from apoptosis through induction of insulin-like growth factors. J. Biol. Chem. 277(41), 38239-38244 (2002).
²⁹Cole, M. F. et al., Tcf3 is an integral component of the core regulatory circuitry of embryonic stem cells. Genes Dev. 22(6), 746-755 (2008).
³⁰van der Flier, L. G. et al., Transcription factor achaete scute-like 2 controls intestinal stem cell fate. Cell 136(5), 903-912 (2009).
³¹Korinek, V. et al., Constitutive transcriptional activation by a beta-catenin-Tcf complex in APC−/− colon carcinoma. Science 275, 1784-1787 (1997).
³²Barolo, S., Transgenic Wnt/TCF pathway reporters: all you need is Lef? Oncogene 25(57), 7505-7511 (2006).
³³DasGupta, R. & Fuchs, E., Multiple roles for activated LEF/TCF transcription complexes during hair follicle development and differentiation. Development 126(20), 4557-4568 (1999).
³⁴Maretto, S. et al., Mapping Wnt/beta-catenin signaling during mouse development and in colorectal tumors. Proc. Natl. Acad. Sci. USA 100(6), 3299-3304 (2003).
³⁵Moriyama, A. et al., GFP transgenic mice reveal active canonical Wnt signal in neonatal brain and in adult liver and spleen. Genesis 45(2), 90-100 (2007).
³⁶Currier, N., Chea, K., Hlavacova, M., Sussman, D. J., Seldin, D. C. & Dominguez, I., Dynamic expression of a LEF-EGFP Wnt reporter in mouse development and cancer. Genesis 3, 183-194 (2010).
³⁷Lustig, B. et al., Negative feedback loop of Wnt signaling through upregulation of conductin/axin2 in colorectal and liver tumors. Mol. Cell. Biol. 22(4), 1184-1193 (2002).
³⁸Jho E. H., Zhang, T., Domon, C., Joo, C. K., Freund, J. N., & Costantini, F. Wnt/beta-catenin/Tcf signaling induces the transcription of Axin2, a negative regulator of the signaling pathway. Mol. Cell. Biol. 22(4), 1172-1183 (2002).
³⁹Barker, N. et al., Identification of stem cells in small intestine and colon by marker gene Lgr5. Nature 449, 1003-1008 (2007).
⁴⁰Dorsky, R. I., Sheldahl, L. C. & Moon R. T., A transgenic Lef1/beta-catenin-dependent reporter is expressed in spatially restricted domains throughout zebrafish development. Dev. Biol. 241(2), 229-237 (2002).
⁴¹Hendriksen, J. et al., Plasma membrane recruitment of dephosphorylated beta-catenin upon activation of the Wnt pathway. J. Cell Sci. 121, 1793-1802 (2008).
⁴²van Noort, M. Meeldijk, J., van der Zee, R., Destree, O., & Clevers, H. Wnt signaling controls the phosphorylation status of beta-catenin. J. Biol. Chem. 277(20), 17901-17905 (2002).
⁴³Klapholz-Brown, Z., Walmsley, G. G., Nusse, Y. M., Nusse, R. & Brown, P. O. Transcriptional program induced by Wnt protein in human fibroblasts suggests mechanisms for cell cooperativity in defining tissue microenvironments. PLoS One 2(9), e945 (2007).
⁴⁴Mahmoudi, T. et al., The kinase TNIK is an essential activator of Wnt target genes. EMBO J. 28(21), 3329-3340 (2009).
⁴⁵Mao, J. et al., Low-density lipoprotein receptor-related protein-5 binds to Axin and regulates the canonical Wnt signaling pathway. Mol. Cell 7(4), 801-809 (2001).
⁴⁶Tolwinski, N. S., Wehrli, M., Rives, A., Erdeniz, N., DiNardo, S. & Wieschaus, E. Wg/Wnt signal can be transmitted through arrow/LRP5,6 and Axin independently of Zw3/Gsk3beta activity. Dev. Cell 4(3), 407-418 (2003).
⁴⁷Tamai, K. et al., A mechanism for Wnt coreceptor activation. Mol. Cell 13(1), 149-156 (2004).
⁴⁸Kohler, G. & Milstein, C., Continuous cultures of fused cells secreting antibody of predefined specificity. Nature 256, 495-497 (1975).
⁴⁹Riechmann, L., Clark, M., Waldmann, H., & Winter, G. Reshaping human antibodies for therapy. Nature 332, 323-327 (1988).
⁵⁰U.S. Pat. No. 5,530,101
⁵¹Jones, P. T., Dear, P. H., Foote, J., Neuberger, M. S. & Winter, G. Replacing the complementarity-determining regions in a human antibody with those from a mouse. Nature 321, 522 (1986).
⁵²Queen, C. et al. A humanized antibody that binds to the interleukin 2 receptor. Proc. Natl. Acad. Sci. USA. 86, 10029-10033 (1989).
⁵³Carter, P. et al., Humanization of an anti-p185HER2 antibody for human cancer therapy. Proc. Natl. Acad. Sci. USA 89, 4285 (1992).
⁵⁴Hwang, W. Y. & Foote, J., Immunogenicity of engineered antibodies. Methods 36, 3-10 (2005).
⁵⁵Padlan, E. A., A possible procedure for reducing the immunogenicity of antibody variable domains while preserving their ligand-binding properties. Mol. Immunol. 28, 489-498 (1991).
⁵⁶Tan, P., Mitchell, D. A., Buss, T. N., Holmes, M. A., Anasetti, C. & Foote, J., “Superhumanized” antibodies: reduction of immunogenic potential by complementarity-determining region grafting with human germline sequences: application to an anti-CD28. J. Immunol. 169, 1119-1125 (2002).
⁵⁷Lazar, G. A., Desjarlais, J. R., Jacinto, J., Karki, S. & Hammond, P. W., A molecular immunology approach to antibody humanization and functional optimization. Mol. Immunol. 44, 1986-1998 (2007).
⁵⁸Almagro, J. C. & Fransson, J., Humanization of antibodies. Frontiers in Bioscience 13, 1619-1633 (2008).
⁵⁹WO09852976A1
⁶⁰Jain, M., Kamal, N., Batra, S. K., Engineering antibodies for clinical applications. Trends in Biotechnol. 25, 307-316 (2007).
⁶¹Canfield, S. M. & Morrison, S. L., The binding affinity of human IgG for its high affinity Fc receptor is determined by multiple amino acids in the CH2 domain and is modulated by the hinge region. J. Exp. Med., 173, 1483-1491 (1991).
⁶²Xu, Y., Oomen, R. & Klein, M. H., Residue at position 331 in the IgG1 and IgG4 CH2 domains contributes to their differential ability to bind and activate complement. J. Biol. Chem. 269, 3469-3474 (1994).
⁶³Pulito, V. L. et al., Humanization and molecular modeling of the anti-CD4 monoclonal antibody, OKT4A. J. Immunol. 156, 2840-2850 (1996).
⁶⁴Vander Neut Kolfschoten, M., Anti-inflammatory activity of human IgG4 antibodies by dynamic Fab arm exchange. Science 317, 1554-1557 (2007).
⁶⁵Coloma, M. J. & Morrison S. L., Design and production of novel tetravalent bispecific antibodies. Nature Biotech. 15, 159-163 (1997).
⁶⁶WO 2001/077342
⁶⁷Morrison, S. L., Two heads are better than one. Nature Biotech 25, 1233-1234 (2007).
⁶⁸Holliger, P. & Hudson, P. J., Engineered antibody fragments and the rise of single domains. Nature Biotech. 23, 1126-1136 (2005).
⁶⁹Fischer, N. & Leger, O., Bispecific antibodies: molecules that enable novel therapeutic strategies. Pathobiology 74, 3-14 (2007).
⁷⁰Shen, J., et al., Single variable domain antibody as a versatile building block for the construction of IgG-like bispecific antibodies. Journal of Immunological Methods 318, 65-74 (2007).
⁷¹Wu, C., et al., Simultaneous targeting of multiple disease mediators by a dual-variable-domain immunoglobulin. Nature Biotech. 25, 1290-1297 (2007).
⁷²Lippincott Williams & Wilkins. Remington; The Science and Practice of Pharmacy. University of Sciences in Philadelphia 21st Edition 2005.
⁷³DeWitt, S. H., Kiely, J. S., Stankovic, C. J., Schroeder, M. C., Cody, D. M. & Pavia, M. R., “Diversomers”: an approach to nonpeptide, nonoligomeric chemical diversity. Proc. Natl. Acad. Sci. USA 90, 6909-6913 (1993).
⁷⁴Erb, E., Janda, K. D. & Breener, S., Recursive deconvolution of combinatorial chemical libraries. Proc. Natl. Acad. Sci. USA 91, 11422-11426 (1994).
⁷⁵Zuckermann R. N. et al., Discovery of nanomolar ligands for 7-transmembrane G-protein-coupled receptors from a diverse N-(substituted)glycine peptoid library. J. Med. Chem. 37, 2678-2685 (1994).
⁷⁶Cho, C. Y. et al., An unnatural biopolymer. Science 261, 1303-1305 (1993).
⁷⁷Carell, T., Wintner, E. A., Bashir-Hashemi, A. & Rebek Jr, J. A Novel Procedure for the Synthesis of Libraries Containing Small Organic Molecules. Angew. Chem. Int. Ed. Engl. 33, 2059-2061 (1994).
⁷⁸Carell, T., Wintner, E. A., Rebek Jr, J. & Sutherland, A. J., A Solution-Phase Screening Procedure for the Isolation of Active Compounds from a Library of Molecules. Angew. Chem. Int. Ed. Engl. 33, 2061-2064 (1994).
⁷⁹Gallop, M. A., Barrett, R. W., Dower, W. J., Fodor, S. P. & Gordon, E. M., Applications of combinatorial technologies to drug discovery. 1. Background and peptide combinatorial libraries. J. Med. Chem. 37, 1233-1251 (1994).
⁸⁰Houghten, R. A., Appel, J. R., Blondelle, S. E., Cuervo, J. H., Dooley, C. T., & Pinilla, C., The use of synthetic peptide combinatorial libraries for the identification of bioactive peptides. Biotechniques 13, 412-421 (1992).
⁸¹Lam, K. S., Salmon, S. E., Hersh, E. M., Hruby, V. J., Kazmierski, W. M. & Knapp R. J., A new type of synthetic peptide library for identifying ligand-binding activity. Nature 354, 82-84 (1991).
⁸²Fodor, S. P., Rava, R. P., Huang, X. C., Pease, A. C., Holmes, C. P. & Adams, C. L., Multiplexed biochemical assays with biological chips. Nature 364, 555-556 (1993).
⁸³U. S. Pat. No. 5,223,409
⁸⁴Cull, M. G., Miller, J. F., Schatz, P. J., Screening for receptor ligands using large libraries of peptides linked to the C terminus of the lac repressor. Proc. Natl. Acad. Sci. USA 89, 1865-1869 (1992).
⁸⁵Scott, J. K. & Smith, G. P., Searching for peptide ligands with an epitope library. Science 249, 386-390 (1990).
⁸⁶Devlin, J. J., Panganiban, L. C. & Devlin, P. E., Random peptide libraries: a source of specific protein binding molecules. Science 249, 404-406 (1990).
⁸⁷Cwirla, S. E., Peters, E. A., Barrett, R. W. & Dower, W. J., Peptides on phage: a vast library of peptides for identifying ligands. Proc. Natl. Acad. Sci. USA 87, 6378-6382 (1990).
⁸⁸Felici, F., Castagnoli, L., Musacchio, A., Jappelli, R. & Cesareni, G., Selection of antibody ligands from a large library of oligopeptides expressed on a multivalent exposition vector. J. Mol. Biol. 222, 301-310 (1991).
⁸⁹Sjölander, S. & Urbaniczky, C., Integrated fluid handling system for biomolecular interaction analysis. Anal. Chem. 63, 2338-2345 (1991).
⁹⁰U. S. Pat. No. 5,631,169
⁹¹U. S. Pat. No. 4,968,103
⁹²Bauer, A. & Kuster, B., Affinity purification-mass spectrometry. Powerful tools for the characterization of protein complexes. Eur. J. Biochem. 270, 570-578 (2003).
⁹³Nakatani, Y. & Ogryzko, V., Immunoaffinity purification of mammalian protein complexes Methods Enzymol. 370, 430-444 (2003).
⁹⁴Gingras, A. C., Gstaiger, M., Raught, B. & Aebersold, R., Analysis of protein complexes using mass spectrometry. Nature Reviews Molecular Cell Biology 8, 645-654 (2007).
⁹⁵Vermeulen, M., Hubner, N. C., Mann, M., High confidence determination of specific protein-protein interactions using quantitative mass spectrometry. Current Opinion in Biotechnology 19(4), 331-337 (2008).
⁹⁶Van Schoore, G., Mendive, F., Pochet, R. & Vassart, G. Expression pattern of the orphan receptor LGR4/GPR48 gene in the mouse. Histochem Cell Biol 124, 35-50 (2005).
⁹⁷Ireland, H., Houghton, C., Howard, L. & Winton, D. J. Cellular inheritance of a Cre-activated reporter gene to determine Paneth cell longevity in the murine small intestine. Dev Dyn 233, 1332-6 (2005).
⁹⁸Sansom, O. J. et al. Myc deletion rescues Apc deficiency in the small intestine. Nature 446, 676-9 (2007).
⁹⁹Sato, T. et al. Single Lgr5 stem cells build crypt-villus structures in vitro without a mesenchymal niche. Nature 459, 262-5 (2009).
¹⁰⁰van Es, J. H. et al. Notch/gamma-secretase inhibition turns proliferative cells in intestinal crypts and adenomas into goblet cells. Nature 435, 959-63 (2005).
¹⁰¹Barker, N. & Clevers, H. Leucine-rich repeat-containing G-protein-coupled receptors as markers of adult stem cells. Gastroenterology 138, 1681-96 (2010).
¹⁰²Barker, N. et al. Identification of stem cells in small intestine and colon by marker gene Lgr5. Nature 449, 1003-7 (2007).
¹⁰³Barker, N. et al. Lgr5(+ve) stem cells drive self-renewal in the stomach and build long-lived gastric units in vitro. Cell Stem Cell 6, 25-36 (2010).
¹⁰⁴Jaks, V. et al. Lgr5 marks cycling, yet long-lived, hair follicle stem cells. Nat Genet 40, 1291-9 (2008).
¹⁰⁵Snippert, H. J. et al. Lgr6 marks stem cells in the hair follicle that generate all cell lineages of the skin. Science 327, 1385-9 (2010).
¹⁰⁶Van Schoore, G., Mendive, F., Pochet, R. & Vassart, G. Expression pattern of the orphan receptor LGR4/GPR48 gene in the mouse. Histochem Cell Biol 124, 35-50 (2005).
¹⁰⁷Morita, H. et al. Neonatal lethality of LGR5 null mice is associated with ankyloglossia and gastrointestinal distension. Mol Cell Biol 24, 9736-43 (2004).
¹⁰⁸Kato, S. et al. Eye-open at birth phenotype with reduced keratinocyte motility in LGR4 null mice. FEBS Lett 581, 4685-90 (2007).
¹⁰⁹Ireland, H., Houghton, C., Howard, L. & Winton, D. J. Cellular inheritance of a Cre-activated reporter gene to determine Paneth cell longevity in the murine small intestine. Dev Dyn 233, 1332-6 (2005).
¹¹⁰Garcia, M. I. et al. LGR5 deficiency deregulates Wnt signaling and leads to precocious Paneth cell differentiation in the fetal intestine. Dev Biol 331, 58-67 (2009).
¹¹¹van der Flier, L. G., Haegebarth, A., Stange, D. E., van de Wetering, M. & Clevers, H. OLFM4 is a robust marker for stem cells in human intestine and marks a subset of colorectal cancer cells. Gastroenterology 137, 15-7 (2009).
¹¹²Pinto, D., Gregorieff, A., Begthel, H. & Clevers, H. Canonical Wnt signals are essential for homeostasis of the intestinal epithelium. Genes Dev 17, 1709-13 (2003).
¹¹³Kuhnert, F. et al. Essential requirement for Wnt signaling in proliferation of adult small intestine and colon revealed by adenoviral expression of Dickkopf-1. Proc Natl Acad Sci US A 101, 266-71 (2004).
¹¹⁴van Es, J. H. et al. Notch/gamma-secretase inhibition turns proliferative cells in intestinal crypts and adenomas into goblet cells. Nature 435, 959-63 (2005).
¹¹⁵Sansom, O. J. et al. Myc deletion rescues Apc deficiency in the small intestine. Nature 446, 676-9 (2007).
¹¹⁶Sato, T. et al. Single Lgr5 stem cells build crypt-villus structures in vitro without a mesenchymal niche. Nature 459, 262-5 (2009).
¹¹⁷Nakatani, Y. & Ogryzko, V. Immunoaffinity purification of mammalian protein complexes. Methods Enzymol 370, 430-44 (2003).
¹¹⁸Kazanskaya, O. et al. R-Spondin2 is a secreted activator of Wnt/beta-catenin signaling and is required for Xenopus myogenesis. Dev Cell 7, 525-34 (2004).
¹¹⁹Kim, K. A. et al. R-Spondin family members regulate the Wnt pathway by a common mechanism. Mol Biol Cell 19, 2588-96 (2008).
¹²⁰Kim, K. A. et al. Mitogenic influence of human R-spondinl on the intestinal epithelium. Science 309, 1256-9 (2005).
¹²¹Zhao, J. et al. R-spondinl, a novel intestinotrophic mitogen, ameliorates experimental colitis in mice. Gastroenterology 132, 1331-43 (2007).
¹²²Ohkawara, B., Glinka, A. & Niehrs, C. Rspo3 Binds Syndecan 4 and Induces Wnt/PCP Signaling via Clathrin-Mediated Endocytosis to Promote Morphogenesis. Dev Cell 20, 303-14 (2011).
¹²³Nam, J. S., Turcotte, T. J., Smith, P. F., Choi, S. & Yoon, J. K. Mouse cristin/R-spondin family proteins are novel ligands for the Frizzled 8 and LRP6 receptors and activate beta-catenin-dependent gene expression. J Biol Chem 281, 13247-57 (2006).
¹²⁴Wei, Q. et al. R-spondinl is a high affinity ligand for LRP6 and induces LRP6 phosphorylation and beta-catenin signaling. J Biol Chem 282, 15903-11 (2007).
¹²⁵Binnerts, M. E. et al. R-Spondin1 regulates Wnt signaling by inhibiting internalization of LRP6. Proc Natl Acad Sci USA 104, 14700-5 (2007).
¹²⁶Glinka, A. et al. Dickkopf-1 is a member of a new family of secreted proteins and functions in head induction. Nature 391, 357-62 (1998).
¹²⁷Korinek, V. et al. Constitutive transcriptional activation by a beta-catenin-Tcf complex in APC−/− colon carcinoma. Science 275, 1784-7 (1997).
¹²⁸Sato, T. et al. Paneth cells constitute the niche for Lgr5 stem cells in intestinal crypts. Nature 469, 415-8 (2011).
¹²⁹WO2010/016766
¹³⁰ Shevchenko, A., Wilm, M., Vorm, O. & Mann, M. Mass spectrometric sequencing of proteins silver-stained polyacrylamide gels. Anal Chem 68, 850-8 (1996).
¹³¹Raijmakers, R. et al. Automated online sequential isotope labeling for protein quantitation applied to proteasome tissue-specific diversity. Mol Cell Proteomics 7, 1755-62 (2008).
¹³²Frese, C. K. et al. Improved Peptide Identification by Targeted Fragmentation Using CID, HCD and ETD on a LTQ-Orbitrap Velos. J Proteome Res (2011).
¹³³Cox, J. & Mann, M. MaxQuant enables high peptide identification rates, individualized p.p.b.-range mass accuracies and proteome-wide protein quantification. Nat Biotechnol 26, 1367-72 (2008).

1. An agonist of the Wnt pathway which mimics the activity of an Rspondin protein binding to at least one Lgr protein.

2. The agonist of claim 1 wherein the Rspondin protein is Rspondin 1, Rspondin 2, Rspondin 3 or Rspondin 4.

3. The agonist of claim 1 or claim 2, which is not antibody 1D9 and/or does not consist of an Rspondin Furin Domain.

4. The agonist of any one of claims 1 to 3, wherein the at least one Lgr protein is Lgr4, Lgr5 and/or Lgr6.

5. The agonist of any one of claims 1 to 4, wherein the at least one Lgr protein is in a complex with at least one Frizzled receptor and at least one LRP co-receptor.

6. The agonist of claim 5, wherein the Frizzled receptor is at least one of Frizzled 1 to Frizzled 10.

7. The agonist of claim 6, wherein the Frizzled receptor is at least one of Frizzled 5, Frizzled 6 and Frizzled 7.

8. The agonist of any one of claims 5 to 7, wherein the LRP co-receptor is at least one of LRP5 and LRP6.

9. The agonist of any one of claims 5 to 8, wherein the complex further comprises a Wnt protein.

10. The agonist of claim 9, wherein the Wnt protein is Wnt3a.

11. The agonist of claims 9 and 10, wherein the complex further comprises (i) at least one of Lgr4, Lgr5 and Lgr6 and (ii) at least one of Frizzled 5 or Frizzled 6 or Frizzled 7 and (iii) at least one of LRP5 or LRP6.

12. The agonist of any one of the preceding claims, which binds to at least one Lgr protein.

13. The agonist of claim 12, which binds specifically to Lgr4, Lgr5 or Lgr6.

14. The agonist of claim 12, which binds to the extracellular parts, for example one or more extracellular part, or transmembrane regions, for example one or more transmembrane region, of the Lgr protein.

15. The agonist of claim 14, wherein the extracellular parts of the Lgr protein comprises the N-terminal region and/or any of the 17 leucine rich repeats, and/or the CRL region and/or any of the 3 exodomain sequences.

16. The agonist of claim 14 or claim 15, which binds to an epitope within SEQ ID NOs: 2-21, 23-25, 27-29, 31-33, 35-37, 39-58, 60-62, 64-66, 68-70, 72-74, 76-95, 97-99, 101-103, 105-107, 109-111 in the Lgr4, Lgr5 or Lgr6 polypeptide sequence.

17. The agonist of any one of the preceding claims, which binds to a region consisting of or comprising the CRL region of Lgr4, Lgr5 and/or Lgr6, wherein the CRL region is represented by SEQ ID NOs: 20, 57 and/or 94, respectively.

18. The agonist of claim 12, which binds to the Rspondin binding site of the Lgr protein.

19. The agonist of any one of claims 12 to 18, which competes for Rspondin binding.

20. The agonist of any one of claims 12 to 18, which binds to the Lgr protein with a greater affinity and/or avidity than that of Rspondin.

21. The agonist of any one of claims 12 to 18, which binds to the Lgr protein with a Kd equal to or less than about 10⁻⁷M, 10⁻⁸M, 10⁻⁹M, 10⁻¹⁰M, 10⁻¹¹M, 10⁻¹²M, 10⁻¹³M or more.

22. The agonist of any one of the preceding claims, which is a polypeptide, a peptidomimetic, an aptamer or a small molecule.

23. The agonist of any one of the preceding claims, which is a fragment or derivative of Rspondin.

24. The agonist of claim 23, wherein the fragment or derivative of Rspondin comprises or consists of an Rspondin Furin domain, for example selected from SEQ ID NOs: 116, 117, 118, 119, 140 or 143.

25. The agonist of any one of the preceding claims, which is an antibody or fragment thereof.

26. The agonist of any one of the preceding claims, which is free of antagonistic activity.

27. The agonist of any one of the preceding claims, which enhances beta-catenin signalling by a factor of 2×, 5×, 10×, 100×, 1000×, 10000× or more as compared to the activity in the absence of the compound.

28. The agonist of claim 27, which enhances beta-catenin signalling by a factor of 2×, 5×, 10×, 100×, 1000×, 10000× or more as measured in terms of one or more of: Wnt/β-catenin target gene expression, TCF reporter gene expression, beta-catenin stabilization, LRP phosphorylation, and/or Axin translocation from cytoplasm to cell membrane and binding to LRP.

29. The agonist of claim 28, wherein the enhanced beta-catenin signalling is measured in the TOPFlash assay.

30. The agonist of any one of claims 1 to 29, wherein the agonist is antibody 1D9.

31. The agonist of any one of claims 12 to 30, which additionally binds to at least one further target polypeptide or other target molecule.

32. A multi-targeting compound, comprising at least one portion with Rspondin-mimicking activity, and at least one other portion that binds to a further target polypeptide or other target molecule.

33. The multi-targeting compound of claim 32, wherein the at least one portion with Rspondin-mimicking activity comprises:
a) an Rspondin protein; or
b) an agonist according to any one of claims 1 to 31.

34. The multi-targeting compound of claim 33, wherein the Rspondin protein comprises or consists of SEQ ID NO: 112, 113, 114 or 115.

35. The agonist of claim 23 or 24 or a multi-targeting compound of claim 33, wherein the agonist is an Rspondin protein fragment comprising or consisting of an amino acid portion of any one of SEQ ID NOs: 112, 113, 114 or 115 which is more than 50, 100, 150 or 200 consecutive amino acids in length.

36. The agonist of any one of claim 23, 24 or 35 or the multi-targeting compound of claim 33 or 35, wherein the Rspondin fragment comprises or consists of an Rspondin Furin domain, wherein preferably, the Rspondin Furin domain has more than 70, 80, 90, 95%, 98% or 99% identity to SEQ ID NOs: 116, 117, 118, 119, 140 or 143, or is 100% identical thereto.

37. The multi-targeting compound of claim 32 or claim 33, wherein the at least one portion with Rspondin-mimicking activity is an antibody that is specific for one of or more than one of Lgr5, Lgr4 and/or Lgr6.

38. The multi-targeting compound of claim 37, wherein the antibody is antibody 1D9.

39. The multi-targeting compound of any one of claims 32 to 38, wherein said multi-targeting compound is a conjugate or fusion protein.

40. The multi-targeting compound of any one of claims 32 to 39, wherein the at least one other portion that binds to a further polypeptide or other target molecule is a polypeptide, a peptidomimetic, an antibody or a fragment thereof, an aptamer or a small molecule.

41. The multi-targeting compound of any one of claims 32 to 39, wherein the at least one other portion that binds to a further polypeptide or other target molecule binds to a basolateral polypeptide or target molecule on the cell or tissue of interest.

42. The agonist of claim 31 or the multi-targeting compound of any one of claims 32 to 41, wherein said further target polypeptide or other target molecule is selected from an Lgr protein, a Frizzled receptor, or a cell- or tissue-specific marker.

43. The agonist or the multi-targeting compound of claim 42, wherein the tissue-specific marker is specific to liver, pancreas, small-intestine, colon, kidney, heart, lung or hair follicle.

44. The agonist or the multi-targeting compound of claim 43, wherein the cell- or tissue-specific marker is selected from the group consisting of Epcam, CA19, A33, L-cadherin, and dipeptidyl peptidase V.

45. The multi-targeting compound of any one of claims 40 to 44, wherein the at least one other portion that binds to a further polypeptide or other target molecule is an anti-Epcam antibody or fragment thereof conjugated or fused to a furin domain fragment comprising or consisting of the sequence in SEQ ID NO: 141 or SEQ ID NO: 143.

46. The agonist or the multi-targeting compound claim 43, wherein the tissue-specific marker is a cell-surface liver-specific marker.

47. The agonist of claim 31 or the multi-targeting compound of any one of claims 32 to 46, which is a bispecific antibody, including but not limited to BiTEs or Tandabs.

48. A composition comprising (a) one or more agonists or multi-targeting compounds according to any one of the preceding claims, and (b) a suitable and/or pharmaceutically acceptable carrier or diluent.

49. The composition according to claim 48, wherein the one or more agonist may be any combination of agonists that target Lgr4, Lgr5 or Lgr6.

50. The composition according to claim 49, wherein the combination of agonists comprises an Lgr4 antibody and an Lgr5 antibody.

51. The composition of any one of claims 48 to 50, which optionally further comprises an effective amount of at least one compound or protein selected from at least one of: an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, or a nutritional drug.

52. An agonist or multi-targeting compound according to any one of claims 1 to 47 or a composition according to any one of claims 48 to 51 for use in therapy.

53. An agonist, multi-targeting compound or composition according to claim 52 for use in treating patients with tissue loss or damage due to aging or pathological conditions and/or for use in tissue regeneration.

54. A method of treating a patient suffering from tissue loss or damage or damage due to aging or pathological conditions comprising administering an agonist or multi-targeting compound according to any one of claims 1 to 47 or a composition according to any one of claims 48 to 51 to said patient.

55. The agonist, multi-targeting compound or composition according to claim 53 or the method according to claim 54, wherein the cause of tissue loss or damage includes but is not limited to or is selected from the list consisting of: radiation/chemotherapy, mucositis, IBD, short bowel syndrome, hereditary bowel disorders, celiac disease, metabolic diseases, hereditary syndromes, (viral) infections (hepB/C), toxic states, alcoholic liver, fatty liver, cirrhosis, infections, pernicious anemia, ulceration, diabetes, destruction of islet cells, loss of bone mass (osteoporosis), loss of functional skin, loss of hair, loss of functional lung tissue, loss of kidney tissue (for instance acute tubulus necrosis), and loss of sensory cells in the inner ear.

56. A method for enhancing the proliferation of cells comprising supplying an agonist, multi-targeting compound or composition according to any one of claims 1 to 53 or 55 to said cells.

57. The method according to claim 56, comprising supplying an antibody to said cells, wherein optionally the antibody is antibody 1D9.

58. The method according to claim 56, comprising supplying a multi-targeting compound to said cells, wherein optionally the multi-targeting compound comprises or consists of an anti-Epcam antibody linked to Rspondin1-4 or a Furin domain fragment according to any one of claims 34-36 or 45.

59. The method of claim 58 further comprising supplying the cells with a growth factor.

60. A method for tissue regeneration of damaged tissue comprising administering an agonist, multi-targeting compound or composition according to any one of claims 1 to 53 or 55 to said damaged tissue.

61. The method of any one of claims 56 to 60, wherein the method is carried out in vivo, ex vivo, or in vitro.

62. A method for identifying an agonist of the Wnt pathway, said method comprising:
a) contacting a complex comprising at least one Lgr protein, at least one Frizzled receptor and at least one LRP protein with a candidate compound in the presence of a Wnt protein; and
b) determining the level of Wnt/beta catenin signalling
wherein an increase in the level of Wnt/beta catenin indicates that the candidate compound is an agonist of the Wnt pathway.

63. A bispecific compound, optionally a bi-specific antibody, which binds to both Lgr4 and Lgr5 and inhibits beta-catenin signalling.

64. A method of inhibiting beta-catenin signalling comprising administering the bi-specific compound of claim 63.

65. The method of claim 64, wherein the method is conducted in vitro, in vivo or ex vivo.

66. The method of claim 64, wherein the method is conducted in vivo and the compound is administered to a patient to treat cancer.

67. A combination of two or all of i) an inhibitor of Lgr5, ii) an inhibitor of Lgr4 and iii) an inhibitor of Lgr6 for use in treating cancer wherein said two or all of i) an inhibitor of Lgr5, ii) an inhibitor of Lgr4 and iii) an inhibitor of Lgr6 are for sequential, simultaneous or separate administration.

68. A method of treating cancer comprising administering two or all of i) an inhibitor of Lgr5, ii) an inhibitor of Lgr4 and iii) an inhibitor of Lgr6 wherein said two or all of i) an inhibitor of Lgr5, ii) an inhibitor of Lgr4 and iii) an inhibitor of Lgr6 are administered sequentially, simultaneously or separately.

69. A cell culture medium comprising an agonist or multi-targeting compound according to any one of claims 1 to 47.

70. An organoid obtained using the method of any one of claims 56 to 59 and/or the cell culture medium of claim 69.


SEQUENCE LISTING (143 sequences)

SEQ ID NO: 1 - protein, 20 aa, Homo sapiens
MPGPLGLLCFLALGLLGSAG

SEQ ID NO: 2 - protein, 41 aa, Homo sapiens
PSGAAPPLCAAPCSCDGDRRVDCSGKGLTAVPEGLSAFTQA

SEQ ID NO: 3 - protein, 24 aa, Homo sapiens
LDISMNNITQLPEDAFKNFPFLEE

SEQ ID NO: 4 - protein, 24 aa, Homo sapiens
LQLAGNDLSFIHPKALSGLKELKV

SEQ ID NO: 5 - protein, 24 aa, Homo sapiens
LTLQNNQLKTVPSEAIRGLSALQS

SEQ ID NO: 6 - protein, 24 aa, Homo sapiens
LRLDANHITSVPEDSFEGLVQLRH

SEQ ID NO: 7 - protein, 24 aa, Homo sapiens
LWLDDNSLTEVPVHPLSNLPTLQA

SEQ ID NO: 8 - protein, 24 aa, Homo sapiens
LTLALNKISSIPDFAFTNLSSLVV

SEQ ID NO: 9 - protein, 25 aa, Homo sapiens
LHLHNNKIRSLSQHCFDGLDNLETL

SEQ ID NO: 10 - protein, 19 aa, Homo sapiens
DLNYNNLGEFPQAIKALPS

SEQ ID NO: 11 - protein, 28 aa, Homo sapiens
LLKELGFHSNSISVIPDGAFDGNPLLRT

SEQ ID NO: 12 - protein, 24 aa, Homo sapiens
IHLYDNPLSFVGNSAFHNLSDLHS

SEQ ID NO: 13 - protein, 23 aa, Homo sapiens
LVIRGASMVQQFPNLTGTVHLES

SEQ ID NO: 14 - protein, 24 aa, Homo sapiens
LTLTGTKISSIPNNLCQEQKMLRT

SEQ ID NO: 15 - protein, 22 aa, Homo sapiens
LDLSYNNIRDLPSFNGCHALEE

SEQ ID NO: 16 - protein, 23 aa, Homo sapiens
ISLQRNQIYQIKEGTFQGLISLR

SEQ ID NO: 17 - protein, 25 aa, Homo sapiens
ILDLSRNLIHEIHSRAFATLGPITN

SEQ ID NO: 18 - protein, 23 aa, Homo sapiens
LDVSFNELTSFPTEGLNGLNQLK

SEQ ID NO: 19 - protein, 24 aa, Homo sapiens
LVGNFKLKEALAAKDFVNLRSLSV

SEQ ID NO: 20 - protein, 78 aa, Homo sapiens
PYAYQCCAFWGCDSYANLNTEDNSLQDHSVAQEKGTADAANVTSTLENEEHSQIIIHCTPSTGAFKPCEYLLGSWMIR

SEQ ID NO: 21 - protein, 22 aa, Homo sapiens
LTVWFIFLVALFFNLLVILTTF

SEQ ID NO: 22 - protein, 9 aa, Homo sapiens
ASCTSLPSS

SEQ ID NO: 23 - protein, 26 aa, Homo sapiens
KLFIGLISVSNLFMGIYTGILTFLDA

SEQ ID NO: 24 - protein, 21 aa, Homo sapiens
VSWGRFAEFGIWWETGSGCKV

SEQ ID NO: 25 - protein, 23 aa, Homo sapiens
AGFLAVFSSESAIFLLMLATVER

SEQ ID NO: 26 - protein, 18 aa, Homo sapiens
SLSAKDIMKNGKSNHLKQ

SEQ ID NO: 27 - protein, 25 aa, Homo sapiens
FRVAALLAFLGATVAGCFPLFHRGE

SEQ ID NO: 28 - protein, 18 aa, Homo sapiens
YSASPLCLPFPTGETPSL

SEQ ID NO: 29 - protein, 25 aa, Homo sapiens
GFTVTLVLLNSLAFLLMAVIYTKLY

SEQ ID NO: 30 - protein, 18 aa, Homo sapiens
CNLEKEDLSENSQSSMIK

SEQ ID NO: 31 - protein, 24 aa, Homo sapiens
HVAWLIFTNCIFFCPVAFFSFAPL

SEQ ID NO: 32 - protein, 11 aa, Homo sapiens
ITAISISPEIM

SEQ ID NO: 33 - protein, 24 aa, Homo sapiens
KSVTLIFFPLPACLNPVLYVFFNP

SEQ ID NO: 34 - protein, 145 aa, Homo sapiens
KFKEDWKLLKRRVTKKSGSVSVSISSQGGCLEQDFYYDCGMYSHLQGNLTVCDCCESFLLTKPVSCKHLIKSHSCPALAVASCQRPEGYWSDCGTQSAHSDYADEEDSFVSDSSDQVQACGRACFYQSRGFPLVRYAYNLPRVKD

SEQ ID NO: 35 - protein, 65 aa, Homo sapiens
PSGAAPPLCAAPCSCDGDRRVDCSGKGLTAVPEGLSAFTQALDISMNNITQLPEDAFKNFPFLEE

SEQ ID NO: 36 - protein, 89 aa, Homo sapiens
PSGAAPPLCAAPCSCDGDRRVDCSGKGLTAVPEGLSAFTQALDISMNNITQLPEDAFKNFPFLEELQLAGNDLSFIHPKALSGLKELKV

SEQ ID NO: 37 - protein, 113 aa, Homo sapiens
PSGAAPPLCAAPCSCDGDRRVDCSGKGLTAVPEGLSAFTQALDISMNNITQLPEDAFKNFPFLEELQLAGNDLSFIHPKALSGLKELKVLTLQNNQLKTVPSEAIRGLSALQS

SEQ ID NO: 38 - protein, 20 aa, Homo sapiens
MDTSRLGVLLSLPVLLQLAT

SEQ ID NO: 39 - protein, 50 aa, Homo sapiens
GGSSPRSGVLLRGCPTHCHCEPDGRMLLRVDCSDLGLSELPSNLSVFTSY

SEQ ID NO: 40 - protein, 24 aa, Homo sapiens
LDLSMNNISQLLPNPLPSLRFLEE

SEQ ID NO: 41 - protein, 24 aa, Homo sapiens
LRLAGNALTYIPKGAFTGLYSLKV

SEQ ID NO: 42 - protein, 24 aa, Homo sapiens
LMLQNNQLRHVPTEALQNLRSLQS

SEQ ID NO: 43 - protein, 24 aa, Homo sapiens
LRLDANHISYVPPSCFSGLHSLRH

SEQ ID NO: 44 - protein, 24 aa, Homo sapiens
LWLDDNALTEIPVQAFRSLSALQA

SEQ ID NO: 45 - protein, 24 aa, Homo sapiens
MTLALNKIHHIPDYAFGNLSSLVV

SEQ ID NO: 46 - protein, 25 aa, Homo sapiens
LHLHNNRIHSLGKKCFDGLHSLETL

SEQ ID NO: 47 - protein, 19 aa, Homo sapiens
DLNYNNLDEFPTAIRTLSN

SEQ ID NO: 48 - protein, 27 aa, Homo sapiens
LKELGFHSNNIRSIPEKAFVGNPSLIT

SEQ ID NO: 49 - protein, 24 aa, Homo sapiens
IHFYDNPIQFVGRSAFQHLPELRT

SEQ ID NO: 50 - protein, 23 aa, Homo sapiens
LTLNGASQITEFPDLTGTANLES

SEQ ID NO: 51 - protein, 24 aa, Homo sapiens
LTLTGAQISSLPQTVCNQLPNLQV

SEQ ID NO: 52 - protein, 22 aa, Homo sapiens
LDLSYNLLEDLPSFSVCQKLQK

SEQ ID NO: 53 - protein, 23 aa, Homo sapiens
IDLRHNEIYEIKVDTFQQLLSLR

SEQ ID NO: 54 - protein, 25 aa, Homo sapiens
SLNLAWNKIAIIHPNAFSTLPSLIK

SEQ ID NO: 55 - protein, 23 aa, Homo sapiens
LDLSSNLLSSFPITGLHGLTHLK

SEQ ID NO: 56 - protein, 24 aa, Homo sapiens
LTGNHALQSLISSENFPELKVIEM

SEQ ID NO: 57 - protein, 88 aa, Homo sapiens
PYAYQCCAFGVCENAYKISNQWNKGDNSSMDDLHKKDAGMFQAQDERDLEDFLLDFEEDLKALHSVQCSPSPGPFKPCEHLLDGWLIR

SEQ ID NO: 58 - protein, 22 aa, Homo sapiens
IGVWTIAVLALTCNALVTSTVF

SEQ ID NO: 59 - protein, 9 aa, Homo sapiens
RSPLYISPI

SEQ ID NO: 60 - protein, 26 aa, Homo sapiens
KLLIGVIAAVNMLTGVSSAVLAGVDA

SEQ ID NO: 61 - protein, 21 aa, Homo sapiens
FTFGSFARHGAWWENGVGCHV

SEQ ID NO: 62 - protein, 23 aa, Homo sapiens
IGFLSIFASESSVFLLTLAALER

SEQ ID NO: 63 - protein, 18 aa, Homo sapiens
GFSVKYSAKFETKAPFSS

SEQ ID NO: 64 - protein, 25 aa, Homo sapiens
LKVIILLCALLALTMAAVPLLGGSK

SEQ ID NO: 65 - protein, 18 aa, Homo sapiens
YGASPLCLPLPFGEPSTM

SEQ ID NO: 66 - protein, 25 aa, Homo sapiens
GYMVALILLNSLCFLMMTIAYTKLY

SEQ ID NO: 67 - protein, 18 aa, Homo sapiens
CNLDKGDLENIWDCSMVK

SEQ ID NO: 68 - protein, 24 aa, Homo sapiens
HIALLLFTNCILNCPVAFLSFSSL

SEQ ID NO: 69 - protein, 11 aa, Homo sapiens
INLTFISPEVI

SEQ ID NO: 70 - protein, 24 aa, Homo sapiens
KFILLVVVPLPACLNPLLYILFNP

SEQ ID NO: 71 - protein, 82 aa, Homo sapiens
HFKEDLVSLRKQTYVWTRSKHPSLMSINSDDVEKQSCDSTQALVTFTSSSITYDLPPSSVPSPAYPVTESCHLSSVAFVPCL

SEQ ID NO: 72 - protein, 74 aa, Homo sapiens
GGSSPRSGVLLRGCPTHCHCEPDGRMLLRVDCSDLGLSELPSNLSVFTSYLDLSMNNISQLLPNPLPSLRFLEE

SEQ ID NO: 73 - protein, 98 aa, Homo sapiens
GGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKV

SEQ ID NO: 74 - protein, 122 aa, Homo sapiens
GGSSPRSGVLLRGCPTHCHCEPDGRMLLRVDCSDLGLSELPSNLSVFTSYLDLSMNNISQLLPNPLPSLRFLEELRLAGNALTYIPKGAFTGLYSLKVLMLQNNQLRHVPTEALQNLRSLQS

SEQ ID NO: 75 - protein, 24 aa, Homo sapiens
MPSPPGLRALWLCAALCASRRAGG

SEQ ID NO: 76 - protein, 46 aa, Homo sapiens
APQPGPGPTACPAPCHCQEDGIMLSADCSELGLSAVPGDLDPLTAY

SEQ ID NO: 77 - protein, 24 aa, Homo sapiens
LDLSMNNLTELQPGLFHHLRFLEE

SEQ ID NO: 78 - protein, 24 aa, Homo sapiens
LRLSGNHLSHIPGQAFSGLYSLKI

SEQ ID NO: 79 - protein, 24 aa, Homo sapiens
LMLQNNQLGGIPAEALWELPSLQS

SEQ ID NO: 80 - protein, 24 aa, Homo sapiens
LRLDANLISLVPERSFEGLSSLRH

SEQ ID NO: 81 - protein, 24 aa, Homo sapiens
LWLDDNALTEIPVRALNNLPALQA

SEQ ID NO: 82 - protein, 24 aa, Homo sapiens
MTLALNRISHIPDYAFQNLTSLVV

SEQ ID NO: 83 - protein, 25 aa, Homo sapiens
LHLHNNRIQHLGTHSFEGLHNLETL

SEQ ID NO: 84 - protein, 19 aa, Homo sapiens
DLNYNKLQEFPVAIRTLGR

SEQ ID NO: 85 - protein, 27 aa, Homo sapiens
LQELGFHNNNIKAIPEKAFMGNPLLQT

SEQ ID NO: 86 - protein, 24 aa, Homo sapiens
IHFYDNPIQFVGRSAFQYLPKLHT

SEQ ID NO: 87 - protein, 23 aa, Homo sapiens
LSLNGAMDIQEFPDLKGTTSLEI

SEQ ID NO: 88 - protein, 30 aa, Homo sapiens
LTLTRAGIRLLPSGMCQQLPRLRVLELSHN

SEQ ID NO: 89 - protein, 24 aa, Homo sapiens
QIEELPSLHRCQKLEEIGLQHNRI

SEQ ID NO: 90 - protein, 22 aa, Homo sapiens
WEIGADTFSQLSSLQALDLSWN

SEQ ID NO: 91 - protein, 23 aa, Homo sapiens
AIRSIHPEAFSTLHSLVKLDLTD

SEQ ID NO: 92 - protein, 18 aa, Homo sapiens
NQLTTLPLAGLGGLMHLK

SEQ ID NO: 93 - protein, 24 aa, Homo sapiens
LKGNLALSQAFSKDSFPKLRILEV

SEQ ID NO: 94 - protein, 91 aa, Homo sapiens
PYAYQCCPYGMCASFFKASGQWEAEDLHLDDEESSKRPLGLLARQAENHYDQDLDELQLEMEDSKPHPSVQCSPTPGPFKPCEYLFESWIG

SEQ ID NO: 95 - protein, 23 aa, Homo sapiens
RLAVWAIVLLSVLCNGLVLLTVF

SEQ ID NO: 96 - protein, 10 aa, Homo sapiens
AGGPVPLPPV

SEQ ID NO: 97 - protein, 27 aa, Homo sapiens
KFVVGAIAGANTLTGISCGLLASVDAL

SEQ ID NO: 98 - protein, 21 aa, Homo sapiens
TFGQFSEYGARWETGLGCRAT

SEQ ID NO: 99 - protein, 22 aa, Homo sapiens
GFLAVLGSEASVLLLTLAAVQC

SEQ ID NO: 100 - protein, 18 aa, Homo sapiens
SVSVSCVRAYGKSPSLGS

SEQ ID NO: 101 - protein, 25 aa, Homo sapiens
VRAGVLGCLALAGLAAALPLASVGE

SEQ ID NO: 102 - protein, 20 aa, Homo sapiens
YGASPLCLPYAPPEGQPAAL

SEQ ID NO: 103 - protein, 25 aa, Homo sapiens
GFTVALVMMNSFCFLVVAGAYIKLY

SEQ ID NO: 104 - protein, 19 aa, Homo sapiens
CDLPRGDFEAVWDCAMVRH

SEQ ID NO: 105 - protein, 23 aa, Homo sapiens
VAWLIFADGLLYCPVAFLSFASM

SEQ ID NO: 106 - protein, 11 aa, Homo sapiens
LGLFPVTPEAV

SEQ ID NO: 107 - protein, 25 aa, Homo sapiens
KSVLLVVLPLPACLNPLLYLLFNPH

SEQ ID NO: 108 - protein, 152 aa, Homo sapiens
FRDDLRRLRPRAGDSGPLAYAAAGELEKSSCDSTQALVAFSDVDLILEASEAGRPPGLETYGFPSVTLISCQQPGAPRLEGSHCVEPEGNHFGNPQPSMDGELLLRAEGSTPAGGGLSGGGGFQPSGLALLHTYEFCRYPAQWRPLESRGPV

SEQ ID NO: 109 - protein, 70 aa, Homo sapiens
APQPGPGPTACPAPCHCQEDGIMLSADCSELGLSAVPGDLDPLTAYLDLSMNNLTELQPGLFHHLRFLEE

SEQ ID NO: 110 - protein, 94 aa, Homo sapiens
APQPGPGPTACPAPCHCQEDGIMLSADCSELGLSAVPGDLDPLTAYLDLSMNNLTELQPGLFHHLRFLEELRLSGNHLSHIPGQAFSGLYSLKI

SEQ ID NO: 111 - protein, 118 aa, Homo sapiens
APQPGPGPTACPAPCHCQEDGIMLSADCSELGLSAVPGDLDPLTAYLDLSMNNLTELQPGLFHHLRFLEELRLSGNHLSHIPGQAFSGLYSLKILMLQNNQLGGIPAEALWELPSLQS

SEQ ID NO: 112 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 113 - protein, 243 aa, Homo sapiens
MQFRLFSFALIILNCMDYSHCQGNRWRRSKRASYVSNPICKGCLSCSKDNGCSRCQQKLFFFLRREGMRQYGECLHSCPSGYYGHRAPDMNRCARCRIENCDSCFSKDFCTKCKVGFYLHRGRCFDECPDGFAPLEETMECVEGCEVGHWSEWGTCSRNNRTCGFKWGLETRTRQIVKKPVKDTILCPTIAESRRCKMTMRHCPGGKRTPKAKEKRNKKKKRKLIERAQEQHSVFLATDRANQ

SEQ ID NO: 114 - protein, 272 aa, Homo sapiens
MHLRLISWLFIILNFMEYIGSQNASRGRRQRRMHPNVSQGCQGGCATCSDYNGCLSCKPRLFFALERIGMKQIGVCLSSCPSGYYGTRYPDINKCTKCKADCDTCFNKNFCTKCKSGFYLHLGKCLDNCPEGLEANNHTMECVSIVHCEVSEWNPWSPCTKKGKTCGFKRGTETRVREIIQHPSAKGNLCPPTNETRKCTVQRKKCQKGERGKKGRERKRKKPNKGESKEAIPDSKSLESSKEIPEQRENKQQQKKRKVQDKQKSVSVSTVH

SEQ ID NO: 115 - protein, 234 aa, Homo sapiens
MRAPLCLLLLVAHAVDMLALNRRKKQVGTGLGGNCTGCIICSEENGCSTCQQRLFLFIRREGIRQYGKCLHDCPPGYFGIRGQEVNRCKKCGATCESCFSQDFCIRCKRQFYLYKGKCLPTCPPGTLAHQNTRECQGECELGPWGGWSPCTHNGKTCGSAWGLESRVREAGRAGHEEAATCQVLSESRKCPIQRPCPGERSPGQKKGRKDRRPRKDRKLDRRLDVRPRQPGLQP

SEQ ID NO: 116 - protein, 95 aa, Homo sapiens
ACAKGCELCSEVNGCLKCSPKLFILLERNDIRQVGVCLPSCPPGYFDARNPDMNKCIKCKIEHCEACFSHNFCTKCKEGLYLHKGRCYPACPEGS

SEQ ID NO: 117 - protein, 94 aa, Homo sapiens
ICKGCLSCSKDNGCSRCQQKLFFFLRREGMRQYGECLHSCPSGYYGHRAPDMNRCARCRIENCDSCFSKDFCTKCKVGFYLHRGRCFDECPDGF

SEQ ID NO: 118 - protein, 94 aa, Homo sapiens
GCQGGCATCSDYNGCLSCKPRLFFALERIGMKQIGVCLSSCPSGYYGTRYPDINKCTKCKADCDTCFNKNFCTKCKSGFYLHLGKCLDNCPEGL

SEQ ID NO: 119 - protein, 93 aa, Homo sapiens
NCTGCIICSEENGCSTCQQRLFLFIRREGIRQYGKCLHDCPPGYFGIRGQEVNRCKKCGATCESCFSQDFCIRCKRQFYLYKGKCLPTCPPGT

SEQ ID NO: 120 - protein, 57 aa, Homo sapiens
MSEWSPWGPCSKKQQLCGFRRGSEERTRRVLHAPVGDHAACSDTKETRRCTVRRVPC

SEQ ID NO: 121 - protein, 57 aa, Homo sapiens
VGHWSEWGTCSRNNRTCGFKWGLETRTRQIVKKPVKDTILCPTIAESRRCKMTMRHC

SEQ ID NO: 122 - protein, 57 aa, Homo sapiens
VSEWNPWSPCTKKGKTCGFKRGTETRVREIIQHPSAKGNLCPPTNETRKCTVQRKKC

SEQ ID NO: 123 - protein, 55 aa, Homo sapiens
LGPWGGWSPCTHNGKTCGSAWGLESRVREAGRAGHEEAATCQVLSESRKCPIQRP

SEQ ID NO: 124 - protein, 53 aa, Homo sapiens
KRRKGGQGRRENANRNLARKESKEAGAGSRRRKGQQQQQQQGTVGPLTSAGPA

SEQ ID NO: 125 - protein, 36 aa, Homo sapiens
RTPKAKEKRNKKKKRKLIERAQEQHSVFLATDRANQ

SEQ ID NO: 126 - protein, 62 aa, Homo sapiens
RGKKGRERKRKKPNKGESKEAIPDSKSLESSKEIPEQRENKQQQKKRKVQDKQKSVSVSTVH

SEQ ID NO: 127 - protein, 35 aa, Homo sapiens
RSPGQKKGRKDRRPRKDRKLDRRLDVRPRQPGLQP

SEQ ID NO: 128 - protein, 263 aa, Homo sapiens
MRLGLCVVALVLSWTHLTISSRGIKGKRQRRISAEGSQACAKGCELCSEINGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 129 - protein, 118 aa, Artificial Sequence
  VH of Lgr5 antibody 1D9
EVKLQESGAALVKPGASVKLSCKASGYTFTDYYIHWVKQSHGKSLEWIGYINPNSGYTNYNEKFKSKATLTVDKSTNTAYMELSRLTSEDSATYYCTRFGSYWYFDFWGQGTTVTVSS

SEQ ID NO: 130 - DNA, 354 bp, Artificial Sequence
  VH Lgr5 Antibody 1D9
caggtgaagctgcaggagtctggggctgcactggtgaagcctggggcctctgtgaagttgtcttgcaaagcttctggttatacattcactgactactatatacactgggtgaagcagagtcatggaaagagccttgagtggattgggtatattaatcctaacagtggttatactaactacaatgaaaagttcaagagcaaggccacattgactgtagacaaatccaccaatacagcctatatggagcttagcagattgacatctgaggactctgcaacctattactgtacaagatttgggagctactggtactttgacttctggggccaagggaccacggtcaccgtctcctca

SEQ ID NO: 131 - protein, 105 aa, Artificial Sequence
  VL Lgr5 antibody 1D9
MTQSPTSMSISIGDRVTMNCKASQNVDSNVDWYQQKTGQSPKLLIYKASNRYTGVPDRFTGSGSGTDFTFTISNMQAEDLAVYYCMQSNSYPLTFGSGTKLEIKR

SEQ ID NO: 132 - DNA, 324 bp, Artificial Sequence
  VL Lgr5 antibody  1D9
gacattgtgatgactcagtctcccacatccatgtccatatcaataggagacagggtcaccatgaactgcaaggccagtcaaaatgtggattctaatgtagactggtaccaacagaaaacagggcagtctcctaaactgcttatctacaaagcatccaaccggtacacgggagtccctgatcgcttcacaggcagtggatctggaacagatttcactttcaccatcagcaacatgcaggctgaagacctggctgtttattactgtatgcagtctaactcctatccgctcacgttcggttctgggaccaagctggagatcaaacgg

SEQ ID NO: 133 - protein, 8 aa, Artificial Sequence
  1D9 VH CDR1
GYTFTDYY

SEQ ID NO: 134 - protein, 8 aa, Artificial Sequence
  1D9 VH CDR2
INPNSGYT

SEQ ID NO: 135 - protein, 11 aa, Artificial Sequence
  1D9 VH CDR3
TRFGSYWYFDF

SEQ ID NO: 136 - protein, 6 aa, Artificial Sequence
  1D9 VL CDR1
QNVDSN

SEQ ID NO: 137 - protein, 3 aa, Artificial Sequence
  1D9 VL CDR2
KAS

SEQ ID NO: 138 - protein, 9 aa, Artificial Sequence
  1D9 VL CDR3
MQSNSYPLT

SEQ ID NO: 139 - protein, 210 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQ

SEQ ID NO: 140 - protein, 146 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPA

SEQ ID NO: 141 - protein, 118 aa, Artificial Sequence
  hRspo1 with articficial peptide linker attached
GGSGGSISAEGSQACAKGCELCSEVNGCLKCSPKLFILLERNDIRQVGVCLPSCPPGYFARNPDMNKCIKCKIEHCEACFSHNFCTKCKEGLYLHKGRCYPACPEGSSAANGTMECSS

SEQ ID NO: 142 - protein, 6 aa, Artificial Sequence
  Peptide linker
GGSGGS

SEQ ID NO: 143 - protein, 100 aa, Artificial Sequence
  Furin repeats in hRspo1 of SEQ ID NO 141
AEGSQACAKGCELCSEVNGCLKCSPKLFILLERNDIRQVGVCLPSCPPGYFARNPDMNKCIKCKIEHCEACFSHNFCTKCKEGLYLHKGRCYPACPEGSS